(19) United States
(12) Patent Application Publication
(10) Pub. No.: US 2001/0039343 A1
(43) Pub. Date: Nov. 8, 2001

(54) Process to intermediates for biologically active compounds

(76) Inventors: Mark Joseph Mulvihill, Ambler, PA (US); James Joseph Gallagher, Fort Washington, PA (US); Brian Scott MacDougall, Penndel, PA (US); Damian Gerard Weaver, Lansdale, PA (US)

Correspondence Address: ROHM AND HAAS COMPANY, PATENT DEPARTMENT, 100 INDEPENDENCE MALL WEST, PHILADELPHIA, PA, 19106-2399, US

(21) Appl. No.: 09/804,704

(22) Filed: Mar. 13, 2001

Related U.S. Application Data
(63) Continuation of application No. PCT/US01/00652, filed on Jan. 26, 2001.
(63) Continuation-in-part of application No. 09/493,865, filed on Jan. 28, 2000.
(60) Provisional application No. 60/178,878, filed on Jan. 28, 2000.

(51) Int. Cl. C07D 241/18; C07D 239/34; C07D 213/30

(52) U.S. Cl. 544/315; 544/239; 544/408; 546/290; 560/129

(57) ABSTRACT

This invention relates to a process for the manufacture of compounds which are useful to make either pesticides that can be used as fungicides, herbicides, insecticides, rodenticides or biocides, or pharmaceutical compounds for both human and veterinary application.

This application claims priority to PCT/US01/00652 filed on Jan. 26, 2001 which in turn claims domestic priority to US 60/178,878 and US 09/493,865, both filed Jan. 28, 2000.

This invention relates to a process for the manufacture of intermediate compounds which are useful to make enhanced propertied pesticides that can be used as fungicides, herbicides, insecticides, rodenticides or biocides. An enhanced propertied pesticide is one which overcomes one or more deficiencies of a traditional pesticide for the end user application. For instance, the traditional pesticide may be slow acting and/or may have a limited spectrum of activity and/or may not possess a sufficiency of rain-fastness in the field to have a good residual activity. In order to be effective against the targeted pest, many traditional pesticides must be applied in an undesirably high use rate from an environmental perspective. This can cause crop damage, especially with herbicides, because of lower than desired selectivity. Frequently the targeted pest builds up a resistance to the applied pesticide and requires larger amounts of it in order to be controlled. Many traditional tank mixes of traditional pesticides cannot be accomplished because of their mutual incompatibility. Additionally, separately applied pesticide combinations arrive at the target pest at different rates, thus muting some of their mutual effectiveness. Some traditional pesticides have adverse water solubilities for their intended application. Many traditional pesticides are of the contact variety and hence possess no systemicity to completely protect the desired crop from the unwanted pest.

The process of the present invention for the manufacture of intermediates allows one to make enhanced propertied pesticides in order to overcome various deficiencies of traditional pesticides. These intermediate compounds are substituted with a moiety comprising a substituent which enhances or changes the properties of the subsequently made pesticidal compound. This substituent can be tailored on the intermediate compound in order to increase the rate of desired pesticidal action, to increase residual control against the pest, to decrease the overall use rate of the pesticide, increase the selectivity of the pesticide, change the water solubility of the pesticide, and increase the systemicity of the pesticide which is made from the intermediate compound. Furthermore, the substituent on the intermediate compound may optionally comprise a pesticidal compound which may be the same as or different from the pesticidal compound on which substitution of the intermediate compound occurs. This allows a combination of pesticides to be applied simultaneously as a single compound to the target pest or its locus. The application of such a compound provides many advantages such as a greater spectrum of activity against various pests, an attenuation of the build up of pest resistance since the pest is being controlled with two different modes of action, and the ability to combine two pesticidal compounds which would otherwise be incompatible with one another in a tank mix.

The process of the present invention for the manufacture of these intermediates also allows one subsequently to make enhanced propertied pharmaceutical compounds for both human and veterinary application. The chemical modification of drugs into labile derivatives with enhanced physicochemical properties that enable better transport through biological barriers is a useful approach for improving-drug delivery. This modification can be conveniently practiced on ionizable molecules containing moieties such as a carboxy group, an amino group or a hydroxy group that can be utilized for derivatization in order to modify their ionization at physiological pH and to render desirable partition and solubility properties. A necessary requirement of this approach is that the enhanced propertied drug is non-toxic and, when administered to a warm-blooded animal including a human being, is enzymatically and/or chemically cleaved in such a manner as to release the drug at its target or site of activity, quantitatively and at a desirable rate, while the remaining cleaved moiety remains non-toxic and is metabolized in such a manner that non-toxic metabolic products are produced. It is naturally also desirable that the enhanced propertied drug can be provided without excessive costs in connection with its production, in particular without an appreciable loss of the unmodified drug itself during its production and recovery, since the unmodified drug is usually the more expensive part of the enhanced propertied drug.

Furthermore, the substituent on the intermediate compound may optionally comprise a pharmaceutical compound which may be the same as or different from the pharmaceutical compound on which substitution of the intermediate compound occurs. This allows a combination of pharmaceutical compounds to be applied simultaneously as a single compound to the host. The application of such a compound provides many advantages such as a greater spectrum of activity against the disease being treated and an attenuation of the build up of disease resistance since the disease is being controlled with two different modes of action. This type of enhanced propertied pharmaceutical compound will naturally comprise two different pharmaceutical moieties which are compatible with one another and which can be used without an antagonistic interactive effect upon the host. Such combinations would be apparent to one of ordinary skill in the art.

The intermediate used to react with either the pesticide or the drug in providing the enhanced propertied pesticide or pharmaceutical drug, respectively, should advantageously be stable and still be reasonably reactive. The enhanced propertied pesticides and pharmaceuticals which may be produced using the intermediates of this invention can be collectively termed enhanced propertied biologically active compounds. Such enhanced propertied biologically active compounds may be described by, but not limited to, a compound of Formula (A)

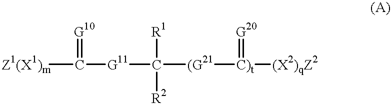

wherein

G¹⁰, G¹¹ and G²⁰ are each independently an oxygen atom or a sulfur atom,

G²¹ is an oxygen atom, a sulfur atom or NR³,

X¹ is an oxygen atom, a sulfur atom, a phosphorous atom or a nitrogen atom attached to Z¹,

X² is an oxygen atom, a sulfur atom, a phosphorous atom, a nitrogen atom or a carbon atom attached to Z²,

m, q and t are each independently 0 or 1,

Z¹(X¹)ₘ is a biologically active moiety when m is 1 wherein Z¹(X¹)m—H represents the biologically active compound,

Z²(X²)_{q}(C(═G²⁰)G²¹)ₜ is a biologically active moiety when q is 1 wherein Z²(X²)_{q}(C(═G²⁰)G²¹)ₜ—H or Z²(X²)_{q}(C(═G²⁰)G²¹)ₜ represents the biologically active compound,

Z¹(X¹)ₘ, when m is 0, is a hydrogen atom, halo, alkyl, alkylcarbonyloxyalkyl, alkylcarbonyl, hydroxyalkyl, alkylsulfonylalkyl, acetylaminoalkyl, haloalkyl, alkenyl, acetylaminoalkenyl, haloalkenyl, alkynyl, haloalkynyl, cycloalkyl, cycloalkenyl, carboxycycloalkyl, carboxycycloalkenyl, cycloalkylalkyl, cycloalkylalkenyl, cycloalkenylalkyl, cycloalkenylalkenyl, cycloalkylalkynyl, cycloalkenylalkynyl, carboxycycloalkylalkyl, carboxycycloalkylalkenyl, carboxycycloalkenylalkyl, carboxycycloalkenylalkenyl, carboxycycloalkylalkynyl, carboxycycloalkenylalkynyl, heterocyclyl, heterocyclylalkyl, heterocyclylalkenyl, heterocyclylalkynyl, alkoxyalkyl, alkoxyalkoxyalkyl, alkoxyalkenyl, alkoxyalkynyl, alkoxycarbonylalkyl, alkoxycarbonylalkenyl, alkoxycarbonylalkynyl, haloalkoxyalkyl, haloalkoxyalkenyl, haloalkoxyalkynyl, alkylthioalkyl, alkylthioalkenyl, alkylthioalkynyl, haloalkylthioalkyl, haloalkylthioalkenyl, haloalkylthioalkynyl, NR³R⁴, SO₂NR³R⁴, OR³, S(O)ⱼR³, carboxyalkyl, carboxyalkenyl, carboxyalkynyl, aryl, aryl substituted with one or more substituents independently selected from halo, nitro, hydroxy, cyano, thiocyanato, alkyl, alkylsulfonylalkyl, alkenyl, alkynyl, haloalkyl, haloalkenyl, haloalkynyl, alkoxy, haloalkoxy, SO₂NR³R⁴ and NR³R⁴, aralkyl, aralkenyl, aralkynyl, arcycloalkyl, aroxyalkyl, or aralkyl, aralkenyl, aralkynyl, arcycloalkyl, aroxyalkyl substituted with one or more substituents independently selected from halo, nitro, hydroxy, cyano, alkyl, cycloalkyl, alkenyl, alkynyl, haloalkyl, haloalkenyl, haloalkynyl, alkoxy, haloalkoxy, SO₂NR³R⁴ and NR³R⁴, heteroaryl, heteroaryl substituted with one or more substituents independently selected from halo, nitro, hydroxy, cyano, alkyl, alkenyl, alkynyl, haloalkyl, haloalkenyl, haloalkynyl, alkoxy, haloalkoxy, SO₂NR³R⁴ and NR³R⁴, heteroaralkyl, heteroaralkenyl, heteroaralkynyl, or heteroaralkyl, heteroaralkenyl, heteroaralkynyl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, wherein j is 0, 1 or 2,

Z²(X²)^{q} is a hydrogen atom, alkyl, alkylcarbonyloxyalkyl, alkylcarbonyl, alkenylcarbonyl, alkynylcarbonyl, hydroxyalkyl, alkylsulfonylalkyl, acetylaminoalkyl, haloalkyl, alkenyl, acetylaminoalkenyl, haloalkenyl, alkynyl, haloalkynyl, cycloalkyl, cycloalkenyl, carboxycycloalkyl, carboxycycloalkenyl, cycloalkylalkyl, cycloalkylalkenyl, cycloalkenylalkyl, cycloalkenylalkenyl, cycloalkylalkynyl, cycloalkenylalkynyl, carboxycycloalkylalkyl, carboxycycloalkylalkenyl, carboxycycloalkenylalkyl, carboxycycloalkenylalkenyl, carboxycycloalkylalkynyl, carboxycycloalkenylalkynyl, heterocyclyl, heterocyclylalkyl, heterocyclylalkenyl, heterocyclylalkynyl, alkoxyalkyl, alkoxyalkoxyalkyl, alkoxyalkenyl, alkoxyalkynyl, alkoxycarbonylalkyl, alkoxycarbonylalkenyl, alkoxycarbonylalkynyl, haloalkoxyalkyl, haloalkoxyalkenyl, haloalkoxyalkynyl, alkylthioalkyl, alkylthioalkenyl, alkylthioalkynyl, haloalkylthioalkyl, haloalkylthioalkenyl, haloalkylthioalkynyl, NR³R⁴, SO₂NR³R⁴, carboxyalkyl, carboxyalkenyl, carboxyalkynyl, dialkoxyphosphorylalkyl, aryl, aryl substituted with one or more substituents independently selected from halo, nitro, hydroxy, cyano, thiocyanato, alkyl, alkylsulfonylalkyl, alkenyl, alkynyl, haloalkyl, haloalkenyl, haloalkynyl, alkoxy, haloalkoxy, SO₂NR³R⁴ and NR³R⁴, aralkyl, aralkenyl, aralkynyl, arcycloalkyl, aroxyalkyl, or aralkyl, aralkenyl, aralkynyl, arcycloalkyl, aroxyalkyl substituted with one or more substituents independently selected from halo, nitro, hydroxy, cyano, alkyl, cycloalkyl, alkenyl, alkynyl, haloalkyl, haloalkenyl, haloalkynyl, alkoxy, haloalkoxy, SO₂NR³R⁴ and NR³R⁴, heteroaryl, heteroaryl substituted with one or more substituents independently selected from halo, nitro, hydroxy, cyano, alkyl, alkenyl, alkynyl, haloalkyl, haloalkenyl, haloalkynyl, alkoxy, haloalkoxy, SO₂NR³R⁴ and NR³R⁴, heteroaralkyl, heteroaralkenyl, heteroaralkynyl, or heteroaralkyl, heteroaralkenyl, heteroaralkynyl substituted with one or more substituents independently selected from halo, hydroxy, nitro, cyano, alkyl, alkenyl, alkynyl, haloalkyl, haloalkenyl, haloalkynyl, alkoxy, haloalkoxy, SO₂NR³R⁴ and NR³R⁴, alkylcarbonylalkyl, alkenylcarbonylalkyl, alkynylcarbonylalkyl, heterocyclylcarbonyl, heterocyclylcarbonylalkyl, heterocyclyloxycarbonylalkyl, arylcarbonyl, arylcarbonylalkyl, aralkylcarbonyl, aralkylcarbonylalkyl, aroxycarbonylalkyl, aralkoxycarbonylalkyl, heteroarylcarbonyl, heteroarylcarbonylalkyl, heteroaroxycarbonylalkyl, or arylcarbonyl, arylcarbonylalkyl, aralkylcarbonyl, aralkylcarbonylalkyl, aroxycarbonylalkyl, aralkoxycarbonylalkyl, heteroarylcarbonyl, heteroarylcarbonylalkyl, heteroaroxycarbonylalkyl substituted with one or more substituents independently selected from halo, hydroxy, nitro, cyano, alkyl, alkenyl, alkynyl, haloalkyl, haloalkenyl, haloalkynyl, alkoxy, haloalkoxy, SO₂NR³R⁴ and NR³R⁴, and C(═N—G²²)R² when q is 0 and t is 1,

G²² is OR³, OCOR³, S(O)ⱼR³, OS(O)ⱼR³, NR³R⁴, OSO₂NR³R⁴, OP(═O)OR³NR³R⁴, OP(═O)(OR³)₂ or N═CR³R⁴,

j is 0, 1 or 2,

Z²(X²)_{q} is halo, NR³R⁴, {(NR³R⁴R⁵)⁺M⁻}, OR³, S(O)ⱼR³ or SO₂NR³R⁴ when both q and t are 0 wherein M⁻ is halo, hydroxy, alkoxy or the anion of a carboxylic acid and j is 0, 1 or 2, R¹ is

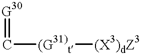

wherein

G³⁰ is an oxygen atom or a sulfur atom,

G³¹ is an oxygen atom, a sulfur atom or NR³,

t′ and d are each independently 0 or 1,

X³ is an oxygen atom, a sulfur atom, a nitrogen atom, a phosphorous atom or a carbon atom attached to Z³ when t′ is 0, a nitrogen atom attached to Z³ when t′ is 1 and G³¹ is NR³, or a carbon atom attached to Z³ when t′ is 1 and G³¹ is an oxygen atom or a sulfur atom,

Z³(X³)_{d}(G³¹)_{t′} is a biologically active moiety when d is 1 wherein Z³(X³)_{d}(G³¹)_{t′}—H represents the biologically active compound,

Z³(X³)_{d}, when d is 0 and t′ is 1, is a hydrogen atom, alkyl, alkylcarbonyloxyalkyl, alkylcarbonyl, alkenylcarbonyl, alkynylcarbonyl, hydroxyalkyl, alkylsulfonylalkyl, acetylaminoalkyl, haloalkyl, alkenyl, acetylaminoalkenyl, haloalkenyl, alkynyl, haloalkynyl, cycloalkyl, cycloalkenyl, carboxycycloalkyl, carboxycycloalkenyl, cycloalkylalkyl, cycloalkylalkenyl, cycloalkenylalkyl, cycloalkenylalkenyl, cycloalkylalkynyl, cycloalkenylalkynyl, carboxycycloalkylalkyl, carboxycycloalkylalkenyl, carboxycycloalkenylalkyl, carboxycycloalkenylalkenyl, carboxycycloalkylalkynyl, carboxycycloalkenylalkynyl, heterocyclyl, heterocyclylalkyl, heterocyclylalkenyl, heterocyclylalkynyl, alkoxyalkyl, alkoxyalkoxyalkyl, alkoxyalkenyl, alkoxyalkynyl, alkoxycarbonylalkyl, alkoxycarbonylalkenyl, alkoxycarbonylalkynyl, haloalkoxyalkyl, haloalkoxyalkenyl, haloalkoxyalkynyl, alkylthioalkyl, alkylthioalkenyl, alkylthioalkynyl, haloalkylthioalkyl, haloalkylthioalkenyl, haloalkylthioalkynyl, carboxyalkyl, carboxyalkenyl, carboxyalkynyl, NR³R⁴, OR³, S(O)ⱼR³, aryl, aryl substituted with one or more substituents independently selected from halo, nitro, hydroxy, cyano, thiocyanato, alkyl, alkylsulfonylalkyl, alkenyl, alkynyl, haloalkyl, haloalkenyl, haloalkynyl, alkoxy, haloalkoxy, SO₂NR³R⁴ and NR³R⁴, aralkyl, aralkenyl, aralkynyl, arcycloalkyl, aroxyalkyl, or aralkyl, aralkenyl, aralkynyl, arcycloalkyl, aroxyalkyl substituted with one or more substituents independently selected from halo, nitro, hydroxy, cyano, alkyl, cycloalkyl, alkenyl, alkynyl, haloalkyl, haloalkenyl, haloalkynyl, alkoxy, haloalkoxy, SO₂NR³R⁴ and NR³R⁴, heteroaryl, heteroaryl substituted with one or more substituents independently selected from halo, nitro, hydroxy, cyano, alkyl, alkenyl, alkynyl, haloalkyl, haloalkenyl, haloalkynyl, alkoxy, haloalkoxy, SO₂NR³R⁴ and NR³R⁴, heteroaralkyl, heteroaralkenyl, heteroaralkynyl, or heteroaralkyl, heteroaralkenyl, heteroaralkynyl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, wherein jis 0, 1 or 2,

Z³(X³)_{d} is halo, NR³R⁴, OR³, N(R³)—N═CR³R⁴, S(O)ⱼR³ or SO₂NR³R⁴ when both d and t′ are 0 and j is 0, 1 or 2,

R² is a hydrogen atom, alkyl, alkenyl, alkynyl, alkoxyalkyl, alkoxyalkenyl, alkoxyalkynyl, alkylthioalkyl, alkylthioalkenyl, alkylthioalkynyl, carboxy, a carboxylate salt, carboxyalkyl, carboxyalkenyl, carboxyalkynyl, alkoxycarbonyl, alkoxycarbonylalkyl, alkoxycarbonylalkenyl, alkoxycarbonylalkynyl, alkylcarbonyl, alkenylcarbonyl, alkynylcarbonyl, cycloalkyl, cycloalkenyl, cycloalkylalkyl, cycloalkenylalkyl, cycloalkylalkenyl, cycloalkenylalkenyl, cycloalkylalkynyl, cycloalkenylalkynyl, heterocyclyl, heterocyclylalkyl, heterocyclylalkenyl, heterocyclylalkynyl, or alkyl, alkenyl, alkynyl, alkoxyalkyl, alkoxyalkenyl, alkoxyalkynyl, alkylthioalkyl, alkylthioalkenyl, alkylthioalkynyl, carboxyalkyl, carboxyalkenyl, carboxyalkynyl, alkoxycarbonyl, alkoxycarbonylalkyl, alkoxycarbonylalkenyl, alkoxycarbonylalkynyl, alkylcarbonyl, alkenylcarbonyl, alkynylcarbonyl, cycloalkyl, cycloalkenyl, cycloalkylalkyl, cycloalkenylalkyl, cycloalkylalkenyl, cycloalkenylalkenyl, cycloalkylalkynyl, cycloalkenylalkynyl, heterocyclyl, heterocyclylalkyl, heterocyclylalkenyl, heterocyclylalkynyl substituted with one or more substituents independently selected from halo, cyano, hydroxy, nitro, SO₂NR³R⁴ and NR³R⁴, aryl or aryl substituted with one or more substituents independently selected from halo, hydroxy, cyano, nitro, alkyl, alkenyl, alkynyl, haloalkyl, haloalkenyl, haloalkynyl, alkoxy, haloalkoxy, carboxy, alkoxycarbonyl, SO₂NR³R⁴ and NR³R⁴, aralkyl, aralkenyl, aralkynyl or aralkyl, aralkenyl, aralkynyl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, arylcarbonyl, aralkylcarbonyl, aralkenylcarbonyl, aralkynylcarbonyl or arylcarbonyl, aralkylcarbonyl, aralkenylcarbonyl, aralkynylcarbonyl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, heteroaryl or heteroaryl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, heteroaralkyl, heteroaralkenyl, heteroaralkynyl or heteroaralkyl, heteroaralkenyl, heteroaralkynyl substituted with one or more substituents independently selected from halo, cyano, hydroxy, nitro, alkyl, alkenyl, alkynyl, haloalkyl, haloalkenyl, haloalkynyl, alkoxy, haloalkoxy, SO₂NR³R⁴ and NR³R⁴, heteroaralkylcarbonyl, heteroaralkenylcarbonyl, heteroaralkynylcarbonyl or heteroaralkylcarbonyl, heteroaralkenylcarbonyl, heteroaralkynylcarbonyl substituted with one or more substituents independently selected from halo, cyano, hydroxy, nitro, alkyl, alkenyl, alkynyl, haloalkyl, haloalkenyl, haloalkynyl, alkoxy, haloalkoxy, SO₂NR³R⁴ and NR³R⁴, or R¹ and R² taken together with the carbon atom to which they are attached form a 5-7 membered saturated or unsaturated ring,

R³, R⁴ and R⁵ are each independently a hydrogen atom, alkyl, cycloalkyl, cycloalkenyl, cycloalkylalkyl, cycloalkylalkenyl, cycloalkylalkynyl, cycloalkenylalkyl, cycloalkenylalkenyl, cycloalkenylalkynyl, carboxyalkyl, carboxyalkenyl, carboxyalkynyl, heterocyclyl, heterocyclylalkyl, heterocyclylalkenyl, heterocyclylalkynyl, alkoxyalkyl, alkenyl, alkynyl, or alkyl, cycloalkyl, cycloalkenyl, cycloalkylalkyl, cycloalkylalkenyl, cycloalkylalkynyl, cycloalkenylalkyl, cycloalkenylalkenyl, cycloalkenylalkynyl, carboxyalkyl, carboxyalkenyl, carboxyalkynyl, heterocyclyl, heterocyclylalkyl, heterocyclylalkenyl, heterocyclylalkynyl, alkoxyalkyl, alkenyl or alkynyl substituted with one or more halo, aryl, aralkyl, aralkenyl, aralkynyl, or aryl, aralkyl, aralkenyl, aralkynyl substituted with one or more substituents independently selected from halo, alkyl, alkenyl, alkynyl, haloalkyl, haloalkenyl, haloalkynyl, alkoxy and haloalkoxy, heteroaryl, heteroaralkyl, heteroaralkenyl, heteroaralkynyl, or heteroaryl, heteroaralkyl, heteroaralkenyl, heteroaralkynyl substituted with one or more substituents independently selected from halo, alkyl, alkenyl, alkynyl, haloalkyl, haloalkenyl, haloalkynyl, alkoxy and haloalkoxy, or R³ and R⁴ taken together with the nitrogen atom to which they are attached form a 5- or 6-membered saturated or unsaturated heterocyclic ring, or

the biologically active acceptable salts, isomers, tautomers, enantiomers and mixtures thereof.

Thus, it is highly desirable that an economic process be developed which provides such ubiquitous intermediates.

U.S. Pat. No. 4,916,230 and 5,401,868 disclose processes for intermediates used to make drugs; however, the process of the present invention leading to intermediates used for enhanced propertied biologically active compounds is neither disclosed nor suggested.

A first embodiment of this invention relates to a process leading to a compound of formula (V)

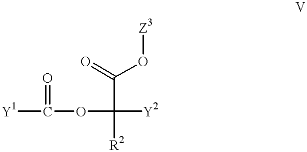

comprising the step of reacting, in the presence of a suitable solvent and a suitable catalyst, a compound of formula (VII)

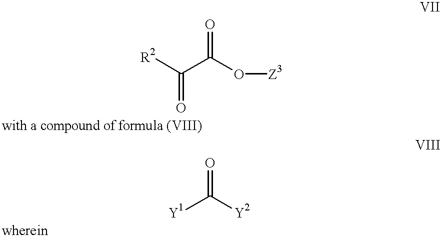

wherein

Z³ is alkyl, alkylcarbonyloxyalkyl, hydroxyalkyl, alkylsulfonylalkyl, acetylaminoalkyl, haloalkyl, alkenyl, acetylaminoalkenyl, haloalkenyl, alkynyl, haloalkynyl, cycloalkyl, cycloalkenyl, carboxycycloalkyl, carboxycycloalkenyl, cycloalkylalkyl, cycloalkylalkenyl, cycloalkenylalkyl, cycloalkenylalkenyl, cycloalkylalkynyl, cycloalkenylalkynyl, carboxycycloalkylalkyl, carboxycycloalkylalkenyl, carboxycycloalkenylalkyl, carboxycycloalkenylalkenyl, carboxycycloalkylalkynyl, carboxycycloalkenylalkynyl, heterocyclyl, heterocyclylalkyl, heterocyclylalkenyl, heterocyclylalkynyl, alkoxyalkyl, alkoxyalkoxyalkyl, alkoxyalkenyl, alkoxyalkynyl, alkoxycarbonylalkyl, alkoxycarbonylalkenyl, alkoxycarbonylalkynyl, haloalkoxyalkyl, haloalkoxyalkenyl, haloalkoxyalkynyl, alkylthioalkyl, alkylthioalkenyl, alkylthioalkynyl, haloalkylthioalkyl, haloalkylthioalkenyl, haloalkylthioalkynyl, carboxyalkyl, carboxyalkenyl, carboxyalkynyl, SiR³R⁴R⁵, aryl,arylsubstituted with one or more substituents independently selected from halo, nitro, hydroxy, cyano, thiocyanato, alkyl, alkylsulfonylalkyl, alkenyl, alkynyl, haloalkyl, haloalkenyl, haloalkynyl, alkoxy, haloalkoxy, SO₂NR³R⁴ and NR³R⁴, aralkyl, aralkenyl, aralkynyl, arcycloalkyl, aroxyalkyl, or aralkyl, aralkenyl, aralkynyl, arcycloalkyl, aroxyalkyl substituted with one or more substituents independently selected from halo, nitro, hydroxy, cyano, alkyl, cycloalkyl, alkenyl, alkynyl, haloalkyl, haloalkenyl, haloalkynyl, alkoxy, haloalkoxy, SO₂NR³R⁴ and NR³R⁴, heteroaryl, heteroaryl substituted with one or more substituents independently selected from halo, nitro, hydroxy, cyano, alkyl, alkenyl, alkynyl, haloalkyl, haloalkenyl, haloalkynyl, alkoxy, haloalkoxy, SO₂NR³R⁴ and NR³R⁴, heteroaralkyl, heteroaralkenyl, heteroaralkynyl, or heteroaralkyl, heteroaralkenyl, heteroaralkynyl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴,

R² is a hydrogen atom, alkyl, alkenyl, alkynyl, alkoxyalkyl, alkoxyalkenyl, alkoxyalkynyl, alkylthioalkyl, alkylthioalkenyl, alkylthioalkynyl, carboxy, a carboxylate salt, carboxyalkyl, carboxyalkenyl, carboxyalkynyl, alkoxycarbonyl, alkoxycarbonylalkyl, alkoxycarbonylalkenyl, alkoxycarbonylalkynyl, alkylcarbonyl, alkenylcarbonyl, alkynylcarbonyl, cycloalkyl, cycloalkenyl, cycloalkylalkyl, cycloalkenylalkyl, cycloalkylalkenyl, cycloalkenylalkenyl, cycloalkylalkynyl, cycloalkenylalkynyl, heterocyclyl, heterocyclylalkyl, heterocyclylalkenyl, heterocyclylalkynyl, or alkyl, alkenyl, alkynyl, alkoxyalkyl, alkoxyalkenyl, alkoxyalkynyl, alkylthioalkyl, alkylthioalkenyl, alkylthioalkynyl, carboxyalkyl, carboxyalkenyl, carboxyalkynyl, alkoxycarbonyl, alkoxycarbonylalkyl, alkoxycarbonylalkenyl, alkoxycarbonylalkynyl, alkylcarbonyl, alkenylcarbonyl, alkynylcarbonyl, cycloalkyl, cycloalkenyl, cycloalkylalkyl, cycloalkenylalkyl, cycloalkylalkenyl, cycloalkenylalkenyl, cycloalkylalkynyl, cycloalkenylalkynyl, heterocyclyl, heterocyclylalkyl, heterocyclylalkenyl, heterocyclylalkynyl substituted with one or more substituents independently selected from halo, cyano, hydroxy, nitro, SO₂NR³R⁴ and NR³R⁴, aryl or aryl substituted with one or more substituents independently selected from halo, hydroxy, cyano, nitro, alkyl, alkenyl, alkynyl, haloalkyl, haloalkenyl, haloalkynyl, alkoxy, haloalkoxy, carboxy, alkoxycarbonyl, SO₂NR³R⁴ and NR³R⁴, aralkyl, aralkenyl, aralkynyl or aralkyl, aralkenyl, aralkynyl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, arylcarbonyl, aralkylcarbonyl, aralkenylcarbonyl, aralkynylcarbonyl or arylcarbonyl, aralkylcarbonyl, aralkenylcarbonyl, aralkynylcarbonyl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, heteroaryl or heteroaryl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, heteroaralkyl, heteroaralkenyl, heteroaralkynyl or heteroaralkyl, heteroaralkenyl, heteroaralkynyl substituted with one or more substituents independently selected from halo, cyano, hydroxy, nitro, alkyl, alkenyl, alkynyl, haloalkyl, haloalkenyl, haloalkynyl, alkoxy, haloalkoxy, SO₂NR³R⁴ and NR³R⁴, heteroaralkylcarbonyl, heteroaralkenylcarbonyl, beteroaralkynylcarbonyl or heteroaralkylcarbonyl, heteroaralkenylcarbonyl, heteroaralkynylcarbonyl substituted with one or more substituents independently selected from halo, cyano, hydroxy, nitro, alkyl, alkenyl, alkynyl, haloalkyl, haloalkenyl, haloalkynyl, alkoxy, haloalkoxy, SO₂NR³R⁴ and NR³R⁴,

R³, R⁴ and R⁵ are each independently a hydrogen atom, alkyl, cycloalkyl, cycloalkenyl, cycloalkylalkyl, cycloalkylalkenyl, cycloalkylalkynyl, cycloalkenylalkyl, cycloalkenylalkenyl, cycloalkenylalkynyl, carboxyalkyl, carboxyalkenyl, carboxyalkynyl, heterocyclyl, heterocyclylalkyl, heterocyclylalkenyl, heterocyclylalkynyl, alkoxyalkyl, alkenyl, alkynyl, or alkyl, cycloalkyl, cycloalkenyl, cycloalkylalkyl, cycloalkylalkenyl, cycloalkylalkynyl, cycloalkenylalkyl, cycloalkenylalkenyl, cycloalkenylalkynyl, carboxyalkyl, carboxyalkenyl, carboxyalkynyl, heterocyclyl, heterocyclylalkyl, heterocyclylalkenyl, heterocyclylalkynyl, alkoxyalkyl, alkenyl or alkynyl substituted with one or more halo, aryl, aralkyl, aralkenyl, aralkynyl, or aryl, aralkyl, aralkenyl, aralkynyl substituted with one or more substituents independently selected from halo, alkyl, alkenyl, alkynyl, haloalkyl, haloalkenyl, haloalkynyl, alkoxy and haloalkoxy, heteroaryl, heteroaralkyl, heteroaralkenyl, heteroaralkynyl, or heteroaryl, heteroaralkyl, heteroaralkenyl, heteroaralkynyl substituted with one or more substituents independently selected from halo, alkyl, alkenyl, alkynyl, haloalkyl, haloalkenyl, haloalkynyl, alkoxy and haloalkoxy, or R³ and R⁴ taken together with the nitrogen atom to which they are attached form a 5- or 6-membered saturated or unsaturated heterocyclic ring, and

Y¹ and Y² are each independently halo or OCC1₃.

In a preferred mode of this first embodiment, Z³ is alkyl, aralkyl or aralkyl substituted with one or more substituents independently selected from halo, alkyl, alkenyl, alkynyl, haloalkyl, haloalkenyl, haloalkynyl, alkoxy and haloalkoxy, and R² is a hydrogen atom.

In a more preferred mode of this first embodiment, Z³ is alkyl or aralkyl.

In another more preferred mode of this first embodiment,

Z³is (C₁-C₂₀)alkyl, (C₁-C₁₀)alkylcarbonyloxy(C₁-Cio)alkyl, hydroxy (C₁-C₂₀)alkyl, (C₁-C₁₀)alkylsulfonyl(C₁-C₁₀)alkyl, acetylamino(C₁-C₁₀)alkyl, halo (C₁-C₂₀)alkyl, (C₂-C₁₀)alkenyl, acetylamino(C₂-C₁₀)alkenyl, halo(C₂-C₁₀)alkenyl, (C₂-C₁₀)alkynyl, halo(C₂-C₁₀)alkynyl, cyclo(C₃-C₈)alkyl, cyclo(C₃-C₈)alkenyl, carboxycyclo(C₃-C₈)alkyl, carboxycyclo(C₃-C₈)alkenyl, cyclo(C₃-C₈)alkyl(C₁-C₁₀)alkyl, cyclo(C₃-C₈)alkyl(C₂-C₁₀)alkenyl, cyclo(C₃-C₈)alkenyl(C₁-C₁₀)alkyl, cyclo(C₃-C₈)alkenyl(C₂-C₁₀)alkenyl, cyclo(C₃-Cs)alkyl(C₂-C₁₀)alkynyl, cyclo (C₃-C₈)alkenyl(C₂-C₁₀)alkynyl, carboxycyclo(C₃-C₈)alkyl(C₁-C₁₀)alkyl, carboxycyclo(C₃-C₈)alkyl(C₂-C₁₀)alkenyl, carboxycyclo(C₃-C₈)alkenyl(C₁-C₁₀)alkyl, carboxycyclo(C₃-C₈)alkenyl(C₂-C₁₀)alkenyl, carboxycyclo(C₃-C₈)alkyl(C₂-C₁₀)alkynyl, carboxycyclo(C₃-C₈)alkenyl(C₂-C₁₀)alkynyl, heterocyclyl, heterocyclyl(C₁C₁₀)alkyl, heterocyclyl(C₂-C₁₀)alkenyl, heterocyclyl(C₂-C₁₀) alkynyl, (C₁-C₁₀)alkoxy(C₁-C₁₀)alkyl, (C₁C₁₀)alkoxy(C₁-C₁₀)alkoxy(C₁-C₁₀) alkyl, (C₁C₁₀)alkoxy(C₂-C₁₀)alkenyl, (C₁C₁₀)alkoxy(C₂-C₁₀)alkynyl, (C₁-C₁₀) alkoxycarbonyl(C₁-C₁₀)alkyl, (C₁-C₁₀)alkoxycarbonyl(C₂-C₁₀)alkenyl, (C₁-C₁₀)alkoxycarbonyl (C₂-C₁₀)alkynyl, halo(C₁-C₁₀)alkoxy(C₁-C₁₀)alkyl, halo(C₁-C₁₀) alkoxy(C₂-C₁₀)alkenyl, halo(C₁-C₁₀)alkoxy(C₂-C₁₀)alkynyl, (C₁-C₁₀) alkylthio(C₁-C₁₀)alkyl, (C₁-C₁₀)alkylthio(C₂-C₁₀)alkenyl, (C₁-C₁₀)alkylthio(C₂-C₁₀) alkynyl, halo(C₁-C₁₀)alkylthio(C₁-C₁₀)alkyl, halo(C₁-C₁₀)alkylthio(C₂-C₁₀) alkenyl, halo(C₁-C₁₀)alkylthio(C₂-C₁₀)alkynyl, carboxy(C₁-C₂₀)alkyl, carboxy(C₂-C₁₀)alkenyl, carboxy(C₂-C₁₀)alkynyl, SiR³R⁴R⁵, aryl, aryl substituted with one or more substituents independently selected from halo, nitro, hydroxy, cyano, thiocyanato, (C₁-C₁₀)alkyl, (C₁-C₁₀)alkylsulfonyl(C₁-C₁₀)alkyl, (C₂-C₁₀) alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, ar(C₁-C₁₀) alkyl, ar(C₂-C₁₀)alkenyl, ar(C₂-C₁₀)alkynyl, arcyclo(C₃-C₈)alkyl, aroxy(C₁-C₁₀)alkyl, or ar (C₁-C₁₀)alkyl, ar(C₂-C₁₀)alkenyl, ar(C₂-C₁₀)alkynyl, arcyclo(C₃-C₈) alkyl, aroxy(C₁-C₁₀)alkyl substituted with one or more substituents independently selected from halo, nitro, hydroxy, cyano, (C₁-C₁₀)alkyl, cycloalkyl, (C₂-C₁₀)alkenyl, (C₂-C₁₀)alkynyl, halo(C₁-C₁₀)alkyl, halo(C₂-C₁₀)alkenyl, halo(C₂-C₁₀) alkynyl, (C₁-C₁₀)alkoxy, halo(C₁-C₁₀)alkoxy, SO₂NR³R⁴ and NR³R⁴, heteroaryl, heteroaryl substituted with one or more substituents independently selected from halo, nitro, hydroxy, cyano, (C₁-C₁₀)alkyl, (C₂-C₁₀)alkenyl, (C₂-C₁₀) alkynyl, halo(C₁-C₁₀)alkyl, halo(C₂-C₁₀)alkenyl, halo(C₂-C₁₀)alkynyl, (C₁-C₁₀) alkoxy, halo(C₁-C₁₀)alkoxy, SO₂NR³R⁴ and NR³R⁴, heteroar(C₁-C₁₀)alkyl, heteroar(C₂-C₁₀)alkenyl, heteroar(C₂-C₁₀)alkynyl, or heteroar(C₁-C₁₀)alkyl, heteroar(C₂-C₁₀)alkenyl, heteroar(C₂-C₁₀)alkynyl substituted with one or more substituents independently selected from halo, hydroxy, cyano, nitro, (C₁-C₁₀) alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴,

R² is a hydrogen atom, (C₁-C₂₀)alkyl, (C₂-C₁₀)alkenyl, (C₂-C₁₀)alkynyl, (C₁-C₁₀) alkoxy(C₁-C₁₀)alkyl, (C₁-C₁₀)alkoxy(C₂-C₁₀)alkenyl, (C₁-C₁₀)alkoxy(C₂-C₁₀) alkynyl, (C₁-C₁₀)alkylthio(C₁-C₁₀)alkyl, (C₁-C₁₀)alkylthio(C₂-C₁₀)alkenyl, (C₁-C₁₀) alkylthio(C₂-C₁₀)alkynyl, carboxy, a carboxylate salt, carboxy(C₁-C₂₀)alkyl, carboxy(C₂-C₂₀)alkenyl, carboxy(C₂-C₂₀)alkynyl, (C₁-C₂₀)alkoxycarbonyl, (C₁-C₁₀) alkoxycarbonyl(C₁-C₁₀)alkyl, (C₁-C₁₀)alkoxycarbonyl(C₂-C₁₀)alkenyl, (C₁-C₁₀) alkoxycarbonyl(C₂-C₁₀)alkynyl, (C₁-C₂₀)alkylcarbonyl, (C₂-C₂₀) alkenylcarbonyl, (C₂-C₂₀)alkynylcarbonyl, cyclo(C₃-C₈)alkyl, cyclo(C₃-C₈) alkenyl, cyclo(C₃-C₈)alkyl(C₁-C₁₀)alkyl, cyclo(C₃-C₈)alkenyl(C₁-C₁₀)alkyl, cyclo(C₃-C₈)alkyl(C₂-C₁₀)alkenyl, cyclo(C₃-C₈)alkenyl(C₂-C₁₀)alkenyl, cyclo (C₃-C₈)alkyl(C₂-C₁₀)alkynyl, cyclo(C₃-C₈)alkenyl(C₂-C₁₀)alkynyl, heterocyclyl, heterocyclyl(C₁-C₁₀)alkyl, heterocyclyl(C₂-C₁₀)alkenyl, heterocyclyl(C₂-C₁₀) alkynyl, or (C₁-C₂₀)alkyl, (C₂-C₁₀)alkenyl, (C₂-C₁₀)alkynyl, (C₁-C₁₀)alkoxy(C₁-C₁₀) alkyl, (C₁-C₁₀)alkoxy(C₂-C₁₀)alkenyl, (C₁-C₁₀)alkoxy(C₂-C₁₀)alkynyl, (C₁-C₁₀) alkylthio(C₁-C₁₀)alkyl, (C₁-C₁₀)alkylthio(C₂-C₁₀)alkenyl, (C₁-C₁₀)alkylthio(C₂-C₁₀) alkynyl, carboxy(C₁-C₂₀)alkyl, carboxy(C₂-C₁₀)alkenyl, carboxy(C₂-C₁₀) alkynyl, (C₁-C₂₀)alkoxycarbonyl, (C₁-C₁₀)alkoxycarbonyl(C₁-C₁₀)alkyl, (C₁-C₁₀) alkoxycarbonyl(C₂-C₁₀)alkenyl, (C₁-C₁₀)alkoxycarbonyl(C₂-C₁₀)alkynyl, (C₁-C₂₀) alkylcarbonyl, (C₂-C₁₀)alkenylcarbonyl, (C₂-C₁₀)alkynylcarbonyl, cyclo(C₃-C₈) alkyl, cyclo(C₃-C₈)alkenyl, cyclo(C₃-C₈)alkyl(C₁-C₁₀)alkyl, cyclo(C₃-C₈) alkenyl(C₁-C₁₀)alkyl, cyclo(C₃-C₈)alkyl(C₂-C₁₀)alkenyl, cyclo(C₃-C₈)alkenyl(C₂-C₁₀) alkenyl, cyclo(C₃-C₈)alkyl(C₂-C₁₀)alkynyl, cyclo(C₃-C₈)alkenyl(C₂-C₁₀)alkynyl, heterocyclyl, heterocyclyl(C₁-C₁₀)alkyl, heterocyclyl(C₂-C₁₀)alkenyl, heterocyclyl(C₂-C₁₀)alkynyl substituted with one or more substituents independently selected from halo, cyano, hydroxy, nitro, SO₂NR³R⁴ and NR³R⁴, aryl or aryl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, carboxy, (C₁-C₄)alkoxycarbonyl, SO₂NR³R⁴ and NR³R⁴, ar(C₁-C₁₀)alkyl, ar(C₂-C₁₀) alkenyl, ar(C₂-C₁₀)alkynyl, or ar(C₁-C₁₀)alkyl, ar(C₂-C₁₀)alkenyl, ar(C₂-C₁₀) alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, arylcarbonyl, ar(C₁-C₁₀)alkylcarbonyl, ar(C₂-C₁₀) alkenylcarbonyl, ar(C₂-C₁₀)alkynylcarbonyl or arylcarbonyl, ar(C₁-C₁₀) alkylcarbonyl, ar(C₂-C₁₀)alkenylcarbonyl, ar(C₂-C₁₀)alkynylcarbonyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀) alkyl, halo(C₂-C₁₀)alkenyl, halo(C₂-C₁₀)alkynyl, (C₁-C₁₀)alkoxy, halo(C₁-C₁₀) alkoxy, SO₂NR³R⁴ and NR³R⁴, heteroaryl, heteroaryl substituted with one or more substituents independently selected from halo, (C₁-C₁₀)alkyl, (C₂-C₁₀) alkenyl, (C₂-C₁₀)alkynyl, halo(C₁-C₁₀)alkyl, halo(C₂-C₁₀)alkenyl, halo(C₂-C₁₀) alkynyl, (C₁-C₁₀)alkoxy, halo(C₁-C₁₀)alkoxy and NR³R⁴, heteroar(C₁-C₁₀)alkyl, heteroar(C₂-C₁₀)alkenyl, heteroar(C₂-C₁₀)alkynyl or heteroar(C₁-C₁₀)alkyl, heteroar(C₂-C₁₀)alkenyl, heteroar(C₂-C₁₀)alkynyl substituted with one or more substituents independently selected from halo, (C₁-C₁₀)alkyl, (C₂-C₁₀)alkenyl, (C₂-C₁₀) alkynyl, halo(C₁-C₁₀)alkyl, halo(C₂-C₁₀)alkenyl, halo(C₂-C₁₀)alkynyl, (C₁-C₁₀) alkoxy, halo(C₁-C₁₀)alkoxy, SO₂NR³R⁴ and NR³R⁴, heteroar(C₁-C₁₀)alkylcarbonyl, heteroar(C₂-C₁₀) alkenylcarbonyl, heteroar(C₂-C₁₀)alkynylcarbonyl or heteroar(C₁-C₁₀)alkylcarbonyl, heteroar(C₂-C₁₀) alkenylcarbonyl, heteroar(C₂-C₁₀)alkynylcarbonyl substituted with one or more substituents independently selected from halo, cyano, hydroxy, nitro, (C₁-C₁₀) alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, or R¹ and R² taken together with the carbon atom to which they are attached form a 5-7 membered saturated or unsaturated ring,

R³, R⁴ and R⁵ are each independently a hydrogen atom, (C₁-C₂₀)alkyl, cyclo(C₃-C₈)alkyl, cyclo(C₃-C₈)alkenyl, cyclo(C₃-C₈)alkyl(C₁-C₁₀)alkyl, cyclo(C₃-C₈) alkyl(C₂-C₁₀)alkenyl, cyclo(C₃-C₈)alkyl(C₂-C₁₀)alkynyl, cyclo(C₃-C₈)alkenyl(C₁-C₁₀) alkyl, cyclo(C₃-C₈)alkenyl(C₂-C₁₀)alkenyl, cyclo(C₃-C₈)alkenyl(C₂-C₁₀)alkynyl, carboxy(C₁-C₂₀)alkyl, carboxy(C₂-C₁₀)alkenyl, carboxy(C₂-C₁₀)alkynyl, heterocyclyl, heterocyclyl(C₁-C₁₀)alkyl, heterocyclyl(C₂-C₁₀)alkenyl, heterocyclyl(C₂-C₁₀)alkynyl, (C₁-C₁₀)alkoxy(C₁-C₁₀)alkyl, (C₂-C₁₀)alkenyl, (C₂-C₁₀) alkynyl, or (C₁-C₁₀)alkyl, cyclo(C₃-C₈)alkyl, cyclo(C₃-C₈)alkenyl, cyclo(C₃-C₈) alkyl(C₁-C₁₀)alkyl, cyclo(C₃-C₈)alkyl(C₂-C₁₀)alkenyl, cyclo(C₃-C₈)alkyl(C₂-C₁₀) alkynyl, cyclo(C₃-C₈)alkenyl(C₁-C₁₀)alkyl, cyclo(C₃-C₈)alkenyl(C₂-C₁₀)alkenyl, cyclo(C₃-C₈)alkenyl(C₂-C₁₀)alkynyl, carboxy(C₁-C₂₀)alkyl, carboxy(C₂-C₁₀)alkenyl, carboxy(C₂-C₁₀)alkynyl, heterocyclyl, heterocyclyl(C₁-C₁₀)alkyl, heterocyclyl(C₂-C₁₀) alkenyl, heterocyclyl(C₂-C₁₀)alkynyl, (C₁-C₁₀)alkoxy(C₁-C₁₀)alkyl, (C₂-C₁₀) alkenyl or (C₂-C₁₀)alkynyl substituted with one or more halo, aryl, ar(C₁-C₁₀) alkyl, ar(C₂-C₁₀)alkenyl, ar(C₂-C₁₀)alkynyl or aryl, ar(C₁-C₁₀)alkyl, ar(C₂-C₁₀) alkenyl, ar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy and halo(C₁-C₁₀)alkoxy, heteroaryl, heteroar(C₁-C₁₀)alkyl, heteroar(C₂-C₁₀)alkenyl, heteroar(C₂-C₁₀)alkynyl or heteroaryl, heteroar(C₁-C₁₀)alkyl, heteroar(C₂-C₁₀) alkenyl, heteroar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy and halo(C₁-C₁₀)alkoxy, or R³ and R⁴ taken together with the nitrogen atom to which they are attached form a 5-or 6-membered saturated or unsaturated heterocyclic ring, and

Y¹ and Y₂ are each independently halo or OCCl₃.

In an even more preferred mode of this first embodiment, Z³ is (C₁-C₂₀) alkyl, ar(C₁-C₁₀)alkyl or ar(C₁-C₁₀)alkyl substituted with one or more substituents independently selected from halo, (C₁-C₁₀)alkyl, (C₂-C₁₀)alkenyl, (C₂-C₁₀) alkynyl, halo(C₁-C₁₀)alkyl, halo(C₂-C₁₀)alkenyl, halo(C₂-C₁₀)alkynyl, (C₁-C₁₀) alkoxy and halo(C₁-C₁₀)alkoxy, and R² is a hydrogen atom.

In yet an even more preferred mode of this first embodiment, Z³ is (C₁-C₁₀) alkyl or ar(C₁-C₆)alkyl.

In all of the modes of this first embodiment, suitable catalysts for use in the process include, but are not limited to, pyridine and the like, thioureas and ureas such as tetra-n-butylurea, phosphoramides such as hexamethylphosphotriamide, substituted amides such as dimethylformamide, quaternary ammonium halides such as tetrabutyl or tributylbenzyl ammonium chloride, arylamines such as N,N-dimethylaminopyridine, N,N-dimethylaniline, tertiary phosphines such as trioctyl phosphine, and alkali metal or alkaline earth metal halides such as cesium or potassium chloride which are used in conjunction with a sequestering agent such as a crown ether (18-crown-6). Pyridine is a preferred catalyst. Suitable solvents for use in the above process include, but are not limited to, ethers such as tetrahydrofuran (THF), glyme, diethyl ether, dioxane and the like, acetonitrile, and chlorinated solvents such as methylene chloride (CH₂Cl₂), carbon tetrachloride (CCl₄) or chloroform (CHCl₃). If desired, mixtures of these solvents may be used. The reaction temperature is between from about −78° C. to about 100° C. Between from about 1 to about 10 equivalents of a compound of formula (VIII) are used per equivalent of a compound of formula (VII).

A second embodiment of this invention relates to a process leading to a compound of formula (III)

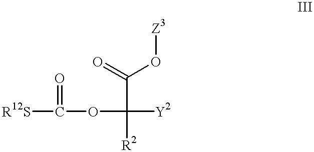

comprising the step of reacting, in the presence of a suitable solvent, a compound of formula (V)

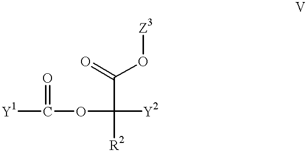

with a compound of formula (VI)

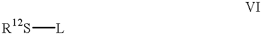

Z³ is alkyl, alkylcarbonyloxyalkyl, hydroxyalkyl, alkylsulfonylalkyl, acetylaminoalkyl, haloalkyl, alkenyl, acetylaminoalkenyl, haloalkenyl, alkynyl, haloalkynyl, cycloalkyl, cycloalkenyl, carboxycycloalkyl, carboxycycloalkenyl, cycloalkylalkyl, cycloalkylalkenyl, cycloalkenylalkyl, cycloalkenylalkenyl, cycloalkylalkynyl, cycloalkenylalkynyl, carboxycycloalkylalkyl, carboxycycloalkylalkenyl, carboxycycloalkenylalkyl, carboxycycloalkenylalkenyl, carboxycycloalkylalkynyl, carboxycycloalkenylalkynyl, heterocyclyl, heterocyclylalkyl, heterocyclylalkenyl, heterocyclylalkynyl, alkoxyalkyl, alkoxyalkoxyalkyl, alkoxyalkenyl, alkoxyalkynyl, alkoxycarbonylalkyl, alkoxycarbonylalkenyl, alkoxycarbonylalkynyl, haloalkoxyalkyl, haloalkoxyalkenyl, haloalkoxyalkynyl, alkylthioalkyl, alkylthioalkenyl, alkylthioalkynyl, haloalkylthioalkyl, haloalkylthioalkenyl, haloalkylthioalkynyl, carboxyalkyl, carboxyalkenyl, carboxyalkynyl, SiR³R⁴R⁵, aryl, aryl substituted with one or more substituents independently selected from halo, nitro, hydroxy, cyano,thiocyanato, alkyl, alkylsulfonylalkyl, alkenyl, alkynyl, haloalkyl, haloalkenyl, haloalkynyl, alkoxy, haloalkoxy, SO₂NR³R⁴ and NR³R⁴, aralkyl, aralkenyl, aralkynyl, arcycloalkyl, aroxyalkyl, or aralkyl, aralkenyl, aralkynyl, arcycloalkyl, aroxyalkyl substituted with one or more substituents independently selected from halo, nitro, hydroxy, cyano, alkyl, cycloalkyl, alkenyl, alkynyl, haloalkyl, haloalkenyl, haloalkynyl, alkoxy, haloalkoxy, SO₂NR³R⁴ and NR³R⁴, heteroaryl, heteroaryl substituted with one or more substituents independently selected from halo, nitro, hydroxy, cyano, alkyl, alkenyl, alkynyl, haloalkyl, haloalkenyl, haloalkynyl, alkoxy, haloalkoxy, SO₂NR³R⁴ and NR³R⁴, heteroaralkyl, heteroaralkenyl, heteroaralkynyl, or heteroaralkyl, heteroaralkenyl, heteroaralkynyl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴,

R² is a hydrogen atom, alkyl, alkenyl, alkynyl, alkoxyalkyl, alkoxyalkenyl, alkoxyalkynyl, alkylthioalkyl, alkylthioalkenyl, alkylthioalkynyl, carboxy, a carboxylate salt, carboxyalkyl, carboxyalkenyl, carboxyalkynyl, alkoxycarbonyl, alkoxycarbonylalkyl, alkoxycarbonylalkenyl, alkoxycarbonylalkynyl, alkylcarbonyl, alkenylcarbonyl, alkynylcarbonyl, cycloalkyl, cycloalkenyl, cycloalkylalkyl, cycloalkenylalkyl, cycloalkylalkenyl, cycloalkenylalkenyl, cycloalkylalkynyl, cycloalkenylalkynyl, heterocyclyl, heterocyclylalkyl, heterocyclylalkenyl, heterocyclylalkynyl, or alkyl, alkenyl, alkynyl, alkoxyalkyl, alkoxyalkenyl, alkoxyalkynyl, alkylthioalkyl, alkylthlioalkenyl, alkylthioalkynyl, carboxyalkyl, carboxyalkenyl, carboxyalkynyl, alkoxycarbonyl, alkoxycarbonylalkyl, alkoxycarbonylalkenyl, alkoxycarbonylalkynyl, alkylcarbonyl, alkenylcarbonyl, alkynylcarbonyl, cycloalkyl, cycloalkenyl, cycloalkylalkyl, cycloalkenylalkyl, cycloalkylalkenyl, cycloalkenylalkenyl, cycloalkylalkynyl, cycloalkenylalkynyl, heterocyclyl, heterocyclylalkyl, heterocyclylalkenyl, heterocyclylalkynyl substituted with one or more substituents independently selected from halo, cyano, hydroxy, nitro, SO₂NR³R⁴ and NR³R⁴, aryl or aryl substituted with one or more substituents independently selected from halo, hydroxy, cyano, nitro, alkyl, alkenyl, alkynyl, haloalkyl, haloalkenyl, haloalkynyl, alkoxy, haloalkoxy, carboxy, alkoxycarbonyl, SO₂NR³R⁴ and NR³R⁴, aralkyl, aralkenyl, aralkynyl or aralkyl, aralkenyl, aralkynyl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, arylcarbonyl, aralkylcarbonyl, aralkenylcarbonyl, aralkynylcarbonyl or arylcarbonyl, aralkylcarbonyl, aralkenylcarbonyl, aralkynylcarbonyl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, heteroaryl or heteroaryl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, heteroaralkyl, heteroaralkenyl, heteroaralkynyl or heteroaralkyl, heteroaralkenyl, heteroaralkynyl substituted with one or more substituents independently selected from halo, cyano, hydroxy, nitro, alkyl, alkenyl, alkynyl, haloalkyl, haloalkenyl, haloalkynyl, alkoxy, haloalkoxy, SO₂NR³R⁴ and NR³R⁴, heteroaralkylcarbonyl, heteroaralkenylcarbonyl, heteroaralkynylcarbonyl or heteroaralkylcarbonyl, heteroaralkenylcarbonyl, heteroaralkynylcarbonyl substituted with one or more substituents independently selected from halo, cyano, hydroxy, nitro, alkyl, alkenyl, alkynyl, haloalkyl, haloalkenyl, haloalkynyl, alkoxy, haloalkoxy, SO₂NR³R⁴ and NR³R⁴,

R³, R⁴ and R⁵ are each independently a hydrogen atom, alkyl, cycloalkyl, cycloalkenyl, cycloalkylalkyl, cycloalkylalkenyl, cycloalkylalkynyl, cycloalkenylalkyl, cycloalkenylalkenyl, cycloalkenylalkynyl, carboxyalkyl, carboxyalkenyl, carboxyalkynyl, heterocyclyl, heterocyclylalkyl, heterocyclylalkenyl, heterocyclylalkynyl, alkoxyalkyl, alkenyl, alkynyl, or alkyl, cycloalkyl, cycloalkenyl, cycloalkylalkyl, cycloalkylalkenyl, cycloalkylalkynyl, cycloalkenylalkyl, cycloalkenylalkenyl, cycloalkenylalkynyl, carboxyalkyl, carboxyalkenyl, carboxyalkynyl, heterocyclyl, heterocyclylalkyl, heterocyclylalkenyl, heterocyclylalkynyl, alkoxyalkyl, alkenyl or alkynyl substituted with one or more halo, aryl, aralkyl, aralkenyl, aralkynyl, or aryl, aralkyl, aralkenyl, aralkynyl substituted with one or more substituents independent ly selected from halo, alkyl, alkenyl, alkynyl, haloalkyl, haloalkenyl, haloalkynyl, alkoxy and haloalkoxy, heteroaryl, heteroaralkyl, heteroaralkenyl, heteroaralkynyl, or heteroaryl, heteroaralkyl, heteroaralkenyl, heteroaralkynyl substituted with one or more substituents independently selected from halo, alkyl, alkenyl, alkynyl, haloalkyl, haloalkenyl, haloalkynyl, alkoxy and haloalkoxy, or R³ and R⁴ taken together with the nitrogen atom to which they are attached form a 5-or 6-membered saturated or unsaturated heterocyclic ring,

R¹² is alkyl, cycloalkyl, cycloalkenyl, cycloalkylalkyl, cycloalkylalkenyl, cycloalkylalkynyl, cycloalkenylalkyl, cycloalkenylalkenyl, cycloalkenylalkynyl, carboxyalkyl, carboxyalkenyl, carboxyalkynyl, heterocyclyl, heterocyclylalkyl, heterocyclylalkenyl, heterocyclylalkynyl, alkoxyalkyl, alkenyl, alkynyl, or alkyl, cycloalkyl, cycloalkenyl, cycloalkylalkyl, cycloalkylalkenyl, cycloalkylalkynyl, cycloalkenylalkyl, cycloalkenylalkenyl, cycloalkenylalkynyl, carboxyalkyl, carboxyalkenyl, carboxyalkynyl, heterocyclyl, heterocyclylalkyl, heterocyclylalkenyl, heterocyclylalkynyl, alkoxyalkyl, alkenyl or alkynyl substituted with one or more halo, aryl or aryl substituted with one or more substituents independently selected from halo, nitro, cyano, haloalkyl, haloalkenyl, haloalkynyl, alkylcarbonyl, alkenylcarbonyl, alkynylcarbonyl, arylcarbonyl, alkylsulfonyl and arylsulfonyl, aralkyl, aralkenyl, aralkynyl, or aralkyl, aralkenyl, aralkynyl substituted with one or more substituents independently selected from halo, alkyl, alkenyl, alkynyl, haloalkyl, haloalkenyl, haloalkynyl, alkoxy and haloalkoxy, heteroaryl, heteroaralkyl, heteroaralkenyl, heteroaralkynyl, or heteroaryl, heteroaralkyl, heteroaralkenyl, heteroaralkynyl substituted with one or more substituents independently selected from halo, alkyl, alkenyl, alkynyl, haloalkyl, haloalkenyl, haloalkynyl, alkoxy and haloalkoxy,

Y¹ and Y² are each independently halo, and

L is a hydrogen atom, a sodium atom or a potassium atom.

In a preferred mode of this second embodiment,

Y¹ is chloro,

Y² is chloro, bromo or iodo,

L is a sodium atom or a potassium atom,

R¹² is alkyl, aralkyl, aryl or heteroaryl, or aralkyl, aryl or heteroaryl substituted with one or more substituents independently selected from halo, alkyl, alkenyl, alkynyl, haloalkyl, haloalkenyl, haloalkynyl, alkoxy and haloalkoxy,

Z³ is alkyl, aryl, aralkyl or aryl or aralkyl substituted with one or more substituents independently selected from halo, alkyl, alkenyl, alkynyl, haloalkyl, haloalkenyl, haloalkynyl, alkoxy and haloalkoxy, and

R² is a hydrogen atom.

In a more preferred mode of this second embodiment R¹² is alkyl, L is sodium and Z³ is alkyl or aralkyl.

In another more preferred mode of this second embodiment,

Z³ is (C₁-C₂₀)alkyl, (C₁-C₁₀)alkylcarbonyloxy(C₁-C₁₀)alkyl, hydroxy(C₁-C₂₀) alkyl, (C₁-C₁₀)alkylsulfonyl(C₁-C₁₀)alkyl, acetylamino(C₁-C₁₀)alkyl, halo(C₁-C₂₀) alkyl, (C₂-C₁₀)alkenyl, acetylamino(C₂-C₁₀)alkenyl, halo(C₂-C₁₀)alkenyl, (C₂-C₁₀) alkynyl, halo(C₂-C₁₀)alkynyl, cyclo(C₃-C₈)alkyl, cyclo(C₃-C₈)alkenyl, carboxycyclo(C₃-C₈)alkyl, carboxycyclo(C₃-C₈)alkenyl, cyclo(C₃-C₈)alkyl(C₁-C₁₀) alkyl, cyclo(C₃-C₈)alkyl(C₂-C₁₀)alkenyl, cyclo(C₃-C₈)alkenyl(C₁-C₁₀)alkyl, cyclo(C₃-C₈)alkenyl(C₂-C₁₀)alkenyl, cyclo(C₃-C₈)alkyl(C₂-C₁₀)alkynyl, cyclo(C₃-C₈) alkenyl(C₂-C₁₀)alkynyl, carboxycyclo(C₃-C₈)alkyl(C₁-C₁₀)alkyl, carboxycyclo(C₃-C₈)alkyl(C₂-C₁₀)alkenyl, carboxycyclo(C₃-C₈)alkenyl(C₁-C₁₀)alkyl, carboxycyclo(C₃-C₈)alkenyl(C₂-C₁₀)alkenyl, carboxycyclo(C₃-C₈)alkyl(C₂-C₁₀) alkynyl, carboxycyclo(C₃-C₈)alkenyl(C₂-C₁₀)alkynyl, heterocyclyl, heterocyclyl(C₁-C₁₀)alkyl, heterocyclyl(C₂-C₁₀)alkenyl, heterocyclyl(C₂-C₁₀) alkynyl, (C₁-C₁₀)alkoxy(C₁-C₁₀)alkyl, (C₁-C₁₀)alkoxy(C₁-C₁₀)alkoxy(C₁-C₁₀) alkyl, (C₁-C₁₀)alkoxy(C₂-C₁₀)alkenyl, (C₁-C₁₀)alkoxy(C₂-C₁₀)alkynyl, (C₁-C₁₀) alkoxycarbonyl(C₁-C₁₀)alkyl, (C₁-C₁₀)alkoxycarbonyl(C₂-C₁₀)alkenyl, (C₁-C₁₀) alkoxycarbonyl(C₂-C₁₀)alkynyl, halo(C₁-C₁₀)alkoxy(C₁-C₁₀)alkyl, halo(C₁-C₁₀) alkoxy(C₂-C₁₀)alkenyl, halo(C₁-C₁₀)alkoxy(C₂-C₁₀)alkynyl, (C₁-C₁₀) alkylthio(C₁-C₁₀)alkyl, (C₁-C₁₀)alkylthio(C₂-C₁₀)alkenyl, (C I-C₁₀)alkylthio(C₂-C₁₀) alkynyl, halo(C₁-C₁₀)alkylthio(C₁-C₁₀)alkyl, halo(C₁-C₁₀)alkylthio(C₂-C₁₀) alkenyl, halo(C₁-C₁₀)alkylthio(C₂-C₁₀)alkynyl, carboxy(C₁-C₂₀)alkyl, carboxy(C₂-C₁₀)alkenyl, carboxy(C₂-C₁₀)alkynyl, SiR³R⁴R⁵, aryl, aryl substituted with one or more substituents independently selected from halo, nitro, hydroxy, cyano, thiocyanato, (C₁-C₁₀)alkyl, (C₁-C₁₀)alkylsulfonyl(C₁-C₁₀)alkyl, (C₂-C₁₀) alkenyl, (C₂-C₁₀)alkynyl, halo(C₁-C₁₀)alkyl, halo(C₂-C₁₀)alkenyl, halo(C₂-C₁₀) alkynyl, (C₁-C₁₀)alkoxy, halo(C₁-C₁₀)alkoxy, SO₂NR³R⁴ and NR³R⁴, ar(C₁-C₁₀) alkyl, ar(C₂-C₁₀)alkenyl, ar(C₂-C₁₀)alkynyl, arcyclo(C₃-C₈)alkyl, aroxy(C₁-C₁₀) alkyl, or ar(C₁-C₁₀)alkyl, ar(C₂-C₁₀)alkenyl, ar(C₂-C₁₀)alkynyl, arcyclo(C₃-C₈) alkyl, aroxy(C₁-C₁₀)alkyl substituted with one or more substituents independently selected from halo, nitro, hydroxy, cyano, (C₁-C₁₀)alkyl, cycloalkyl, (C₂-C₁₀)alkenyl, (C₂-C₁₀)alkynyl, halo(C₁-C₁₀)alkyl, halo(C₂-C₁₀)alkenyl, halo(C₂-C₁₀) alkynyl, (C₁-C₁₀)alkoxy, halo(C₁-C₁₀)alkoxy, SO₂NR³R⁴ and NR³R⁴, heteroaryl, heteroaryl substituted with one or more substituents independently selected from halo, nitro, hydroxy, cyano, (C₁-C₁₀)alkyl, (C₂-C₁₀)alkenyl, (C₂-C₁₀) alkynyl, halo(C₁-C₁₀)alkyl, halo(C₂-C₁₀)alkenyl, halo(C₂-C₁₀)alkynyl, (C₁-C₁₀) alkoxy, halo(C₁-C₁₀)alkoxy, SO₂NR³R⁴ and NR³R⁴, heteroar(C₁-C₁₀)alkyl, heteroar(C₂-C₁₀)alkenyl, heteroar(C₂-C₁₀)alkynyl, or heteroar(C₁-C₁₀)alkyl, beteroar(C₂-C₁₀)alkenyl, heteroar(C₂-C₁₀)alkynyl substituted with one or more substituents independently selected from halo, hydroxy, cyano, nitro, (C₁-C₁₀) alkyl, (C₂-C₁₀)alkenyl, (C₂-C₁₀)alkynyl, halo(C₁-C₁₀)alkyl, halo(C₂-C₁₀)alkenyl, balo(C₂-C₁₀)alkynyl, (C₁-C₁₀)alkoxy, halo(C₁-C₁₀)alkoxy, SO₂NR³R⁴ and NR³R⁴,

R² is a hydrogen atom, (C₁-C₂₀)alkyl, (C₂-C₁₀)alkenyl, (C₂-C₁₀)alkynyl, (C₁-C₁₀) alkoxy(C₁-C₁₀)alkyl, (C₁-C₁₀)alkoxy(C₂-C₁₀)alkenyl, (C₁-C₁₀)alkoxy(C₂-C₁₀) alkynyl, (C₁-C₁₀)alkylthio(C₁-C₁₀)alkyl, (C₁-C₁₀)alkylthio(C₂-C₁₀)alkenyl, (C₁-C₁₀) alkylthio(C₂-C₁₀)alkynyl, carboxy, a carboxylate salt, carboxy(C₁-C₂₀)alkyl, carboxy(C₂-C₂₀)alkenyl, carboxy(C₂-C₂₀)alkynyl, (C₁-C₂₀)alkoxycarbonyl, (C₁-C₁₀) alkoxycarbonyl(C₁-C₁₀)alkyl, (C₁-C₁₀)alkoxycarbonyl(C₂-C₁₀)alkenyl, (C₁-C₁₀) alkoxycarbonyl(C₂-C₁₀)alkynyl, (C₁-C₂₀)alkylcarbonyl, (C₂-C₂₀) alkenylcarbonyl, (C₂-C₂₀)alkynylcarbonyl, cyclo(C₃-C₈)alkyl, cyclo(C₃-C₈) alkenyl, cyclo(C₃-C₈)alkyl(C₁-C₁₀)alkyl, cyclo(C₃-C₈)alkenyl(C₁-C₁₀)alkyl, cyclo(C₃-C₈)alkyl(C₂-C₁₀)alkenyl, cyclo(C₃-C₈)alkenyl(C₂-C₁₀)alkenyl, cyclo(C₃-C₈) alkyl(C₂-C₁₀)alkynyl, cyclo(C₃-C₈)alkenyl(C₂-C₁₀)alkynyl, heterocyclyl, heterocyclyl(C₁-C₁₀)alkyl, heterocyclyl(C₂-C₁₀)alkenyl, heterocyclyl(C₂-C₁₀) alkynyl, or (C₁-C₂₀)alkyl, (C₂-C₁₀)alkenyl, (C₂-C₁₀)alkynyl, (C₁-C₁₀)alkoxy(C₁-C₁₀) alkyl, (C₁-C₁₀)alkoxy(C₂-C₁₀)alkenyl, (C₁-C₁₀)alkoxy(C₂-C₁₀)alkynyl, (C₁-C₁₀) alkylthio(C₁-C₁₀)alkyl, (C₁-C₁₀)alkylthio(C₂-C₁₀)alkenyl, (C₁-C₁₀)alkylthio(C₂-C₁₀) alkynyl, carboxy(C₁-C₂₀)alkyl, carboxy(C₂-C₁₀)alkenyl, carboxy(C₂-C₁₀) alkynyl, (C₁-C₂₀)alkoxycarbonyl, (C₁-C₁₀)alkoxycarbonyl(C₁-C₁₀)alkyl, (C₁-C₁₀) alkoxycarbonyl(C₂-C₁₀)alkenyl, (C₁-C₁₀)alkoxycarbonyl(C₂-C₁₀)alkynyl, (C₁-C₂₀) alkylcarbonyl, (C₂-C₁₀)alkenylcarbonyl, (C₂-C₁₀)alkynylcarbonyl, cyclo(C₃-C₈) alkyl, cyclo(C₃-C₈)alkenyl, cyclo(C₃-C₈)alkyl(C₁-C₁₀)alkyl, cyclo(C₃-C₈) alkenyl(C₁-C₁₀)alkyl, cyclo(C₃-C₈)alkyl(C₂-C₁₀)alkenyl, cyclo(C₃-C₈)alkenyl(C₂-C₁₀) alkenyl, cyclo(C₃-C₈)alkyl(C₂-C₁₀)alkynyl, cyclo(C₃-C₈)alkenyl(C₂-C₁₀)alkynyl, heterocyclyl, heterocyclyl(C₁-C₁₀)alkyl, heterocyclyl(C₂-C₁₀)alkenyl, heterocyclyl(C₂-C₁₀)alkynyl substituted with one or more substituents independently selected from halo, cyano, hydroxy, nitro, SO₂NR³R⁴ and NR³R⁴, aryl or aryl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, carboxy, (C₁-C₄)alkoxycarbonyl, SO₂NR³R⁴ and NR³R⁴, ar(C₁-C₁₀)alkyl, ar(C₂-C₁₀) alkenyl, ar(C₂-C₁₀)alkynyl, or ar(C₁-C₁₀)alkyl, ar(C₂-C₁₀)alkenyl, ar(C₂-C₁₀) alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, arylcarbonyl, ar(C₁-C₁₀)alkylcarbonyl, ar(C₂-C₁₀) alkenylcarbonyl, ar(C₂-C₁₀)alkynylcarbonyl or arylcarbonyl, ar(C1-C₁₀) alkylcarbonyl, ar(C₂-C₁₀)alkenylcarbonyl, ar(C₂-C₁₀)alkynylcarbonyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀) alkyl, halo(C₂-C₁₀)alkenyl, halo(C₂-C₁₀)alkynyl, (C₁-C₁₀)alkoxy, halo(C₁-C₁₀) alkoxy, SO₂NR³R⁴ and NR³R⁴, heteroaryl, heteroaryl substituted with one or more substituents independently selected from halo, (C₁-C₁₀)alkyl, (C₂-C₁₀) alkenyl, (C₂-C₁₀)alkynyl, halo(C₁-C₁₀)alkyl, halo(C₂-C₁₀)alkenyl, halo(C₂-C₁₀) alkynyl, (C₁-C₁₀)alkoxy, halo(C₁-C₁₀)alkoxy and NR³R⁴, heteroar(C₁-C₁₀)alkyl, heteroar(C₂-C₁₀)alkenyl, heteroar(C₂-C₁₀)alkynyl or heteroar(C₁-C₁₀)alkyl, heteroar(C₂-C₁₀)alkenyl, heteroar(C₂-C₁₀)alkynyl substituted with one or more substituents independently selected from halo, (C₁-C₁₀)alkyl, (C₂-C₁₀)alkenyl, (C₂-C₁₀) alkynyl, halo(C₁-C₁₀)alkyl, halo(C₂-C₁₀)alkenyl, halo(C₂-C₁₀)alkynyl, (C₁-C₁₀) alkoxy, halo(C₁-C₁₀)alkoxy, SO₂NR³R⁴ and NR³R⁴, heteroar(C₁-C₁₀) alkylcarbonyl, heteroar(C₂-C₁₀)alkenylcarbonyl, heteroar(C₂-C₁₀) alkynylcarbonyl or heteroar(C₁-C₁₀)alkylcarbonyl, heteroar(C₂-C₁₀) alkenylcarbonyl, heteroar(C₂-C₁₀)alkynylcarbonyl substituted with one or more substituents independently selected from halo, cyano, hydroxy, nitro, (C₁-C₁₀) alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, or R¹ and R² taken together with the carbon atom to which they are attached form a 5-7 membered saturated or unsaturated ring,

R³, R⁴ and R⁵ are each independently a hydrogen atom, (C₁-C₂₀)alkyl, cyclo(C₃-C₈)alkyl, cyclo(C₃-C₈)alkenyl, cyclo(C₃-C₈)alkyl(C₁-C₁₀)alkyl, cyclo(C₃-C₈) alkyl(C₂-C₁₀)alkenyl, cyclo(C₃-C₈)alkyl(C₂-C₁₀)alkynyl, cyclo(C₃-C₈)alkenyl(C₁-C₁₀) alkyl, cyclo(C₃-C₈)alkenyl(C₂-C₁₀)alkenyl, cyclo(C₃-C₈)alkenyl(C₂-C₁₀)alkynyl, carboxy(C₁-C₂₀)alkyl, carboxy(C₂-C₁₀)alkenyl, carboxy(C₂-C₁₀)alkynyl, heterocyclyl, heterocyclyl(C₁-C₁₀)alkyl, heterocyclyl(C₂-C₁₀)alkenyl, heterocyclyl(C₂-C₁₀)alkynyl, (C₁-C₁₀)alkoxy(C₁-C₁₀)alkyl, (C₂-C₁₀)alkenyl, (C₂-C₁₀) alkynyl, or (C₁-C₁₀)alkyl, cyclo(C₃-C₈)alkyl, cyclo(C₃-C₈)alkenyl, cyclo(C₃-C₈) alkyl(C₁-C₁₀)alkyl, cyclo(C₃-C₈)alkyl(C₂-C₁₀)alkenyl, cyclo(C₃-C₈)alkyl(C₂-C₁₀) alkynyl, cyclo(C₃-C₈)alkenyl(C₁-C₁₀)alkyl, cyclo(C₃-C₈)alkenyl(C₂-C₁₀)alkenyl, cyclo(C₃-C₈)alkenyl(C₂-C₁₀)alkynyl, carboxy(C₁-C₂₀)alkyl, carboxy(C₂-C₁₀)alkenyl, carboxy(C₂-C₁₀)alkynyl, heterocyclyl, heterocyclyl(C₁-C₁₀)alkyl, heterocyclyl(C₂-C₁₀) alkenyl, heterocyclyl(C₂-C₁₀)alkynyl, (C₁-C₁₀)alkoxy(C₁-C₁₀)alkyl, (C₂-C₁₀) alkenyl or (C₂-C₁₀)alkynyl substituted with one or more halo, aryl, ar(C₁-C₁₀) alkyl, ar(C₂-C₁₀)alkenyl, ar(C₂-C₁₀)alkynyl or aryl, ar(C₁-C₁₀)alkyl, ar(C₂-C₁₀) alkenyl, ar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy and halo(C₁-C₁₀)alkoxy, heteroaryl, heteroar(C₁-C₁₀)alkyl, heteroar(C₂-C₁₀)alkenyl, heteroar(C₂-C₁₀)alkynyl or heteroaryl, heteroar(C₁-C₁₀)alkyl, heteroar(C₂-C₁₀) alkenyl, heteroar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy and halo(C₁-C₁₀)alkoxy, or R³ and R⁴ taken together with the nitrogen atom to which they are attached form a 5-or 6-membered saturated or unsaturated heterocyclic ring,

R¹² is (C₁-C₂₀)alkyl, cyclo(C₃-C₈)alkyl, cyclo(C₃-C₈)alkenyl, cyclo(C₃-C₈) alkyl(C₁-C₁₀)alkyl, cyclo(C₃-C₈)alkyl(C₂-C₁₀)alkenyl, cyclo(C₃-C₈)alkyl(C₂-C₁₀) alkynyl, cyclo(C₃-C₈)alkenyl(C₁-C₁₀)alkyl, cyclo(C₃-C₈)alkenyl(C₂-C₁₀)alkenyl, cyclo(C₃-C₈)alkenyl(C₂-C₁₀)alkynyl, carboxy(C₁-C₂₀)alkyl, carboxy(C₂-C₂₀)alkenyl, carboxy(C₂-C₂₀)alkynyl, heterocyclyl, heterocyclyl(C₁-C₁₀)alkyl, heterocyclyl(C₂-C₁₀) alkenyl, heterocyclyl(C₂-C₁₀)alkynyl, (C₁-C₁₀)alkoxy(C₁-C₁₀)alkyl, (C₂-C₂₀) alkenyl, (C₂-C₂₀)alkynyl, or (C₁-C₂₀)alkyl, cyclo(C₃-C₈)alkyl, cyclo(C₃-C₈) alkenyl, cyclo(C₃-C₈)alkyl(C₁-C₁₀)alkyl, cyclo(C₃-C₈)alkyl(C₂-C₁₀)alkenyl, cyclo(C₃-C₈)alkyl(C₂-C₁₀)alkynyl, cyclo(C₃-C₈)alkenyl(C₁-C₁₀)alkyl, cyclo(C₃-C₈) alkenyl(C₂-C₁₀)alkenyl, cyclo(C₃-C₈)alkenyl(C₂-C₁₀)alkynyl, carboxy(C₁-C₂₀) alkyl, carboxy(C₂-C₂₀)alkenyl, carboxy(C₂-C₂₀)alkynyl, heterocyclyl, heterocyclyl(C₁-C₁₀)alkyl, heterocyclyl(C₂-C₁₀)alkenyl, heterocyclyl(C₂-C₁₀) alkynyl, (C₁-C₁₀)alkoxy(C₁-C₁₀)alkyl, (C₂-C₂₀)alkenyl or (C₂-C₂₀)alkynyl substituted with one or more halo, aryl or aryl substituted with one or more substituents independently selected from halo, nitro, cyano, halo(C₁-C₁₀)alkyl, halo(C₂-C₁₀)alkenyl, halo(C₂-C₁₀)alkynyl, (C₁-C₁₀)alkylcarb onyl, (C₂-C₁₀) alkenylcarbonyl, (C₂-C₁₀)alkynylcarbonyl, arylcarbonyl, (C₁-C₁₀)alkylsulfonyl and arylsulfonyl, ar(C₁-C₁₀)alkyl, ar(C₂-C₁₀)alkenyl, ar(C₂-C₁₀)alkynyl, or ar(C₁-C₁₀) alkyl, ar(C₂-C₁₀)alkenyl, ar(C₂-C₁₀)alkynyl substituted with one or more substituents independently selected from halo, (C₁-C₁₀)alkyl, (C₂-C₁₀)alkenyl, (C₂-C₁₀) alkynyl, halo(C₁-C₁₀)alkyl, halo(C₂-C₁₀)alkenyl, halo(C₂-C₁₀)alkynyl, (C₁-C₁₀) alkoxy and halo(C₁-C₁₀)alkoxy, heteroaryl, heteroar(C₁-C₁₀)alkyl, heteroar(C₂-C₁₀)alkenyl, heteroar(C₂-C₁₀)alkynyl, or heteroaryl, heteroar(C₁-C₁₀) alkyl, heteroar(C₂-C₁₀)alkenyl, heteroar(C₂-C₁₀)alkynyl substituted with one or more substituents independently selected from halo, (C₁-C₁₀)alkyl, (C₂-C₁₀) alkenyl, (C₂-C₁₀)alkynyl, halo(C₁-C₁₀)alkyl, halo(C₂-C₁₀)alkenyl, halo(C₂-C₁₀) alkynyl, (C₁-C₁₀)alkoxy and halo(C₁-C₁₀)alkoxy,

Y¹ and Y² are each independently halo, and

L is a hydrogen atom, a sodium atom or a potassium atom.

In an even more preferred mode of this second embodiment,

Y¹ is chloro,

Y² is chloro or iodo,

L is a sodium atom or a potassium atom,

R¹² is (C₁-C₂₀)alkyl, ar(C₁-C₁₀)alkyl, aryl, heteroaryl or ar(C₁-C₁₀)alkyl, aryl or heteroaryl substituted with one or more substituents independently selected from halo, (C₁-C₁₀)alkyl, (C₂-C₁₀)alkenyl, (C₂-C₁₀)alkynyl, halo(C₁-C₁₀) alkyl, halo(C₂-C₁₀)alkenyl, halo(C₂-C₁₀)alkynyl, (C₁-C₁₀)alkoxy and halo(C₁-C₁₀)alkoxy,

Z³ is (C₁-C₂₀)alkyl, aryl, ar(C₁-C₁₀)alkyl or aryl or ar(C₁-C₁₀)alkyl substituted with one or more substituents independently selected from halo, (C₁-C₁₀) alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy and halo(C₁-C₁₀)alkoxy, and

R² is a hydrogen atom.

In yet an even more preferred mode of this second embodiment R¹² is (C₁-C₁₀) alkyl, L is sodium and Z³ is (C₁-C₁₀)alkyl or ar(C₁-C₆)alkyl.

In all of the modes of this second embodiment, suitable solvents for use in the process include, but are not limited to, ethers such as tetrahydrofuran (THF), glyme, diethyl ether and the like, dimethylformamide (DMF), dimethylsulfoxide (DMSO), acetonitrile, chlorinated solvents such as methylene chloride (CH₂Cl₂) or chloroform (CHCl₃). If desired, mixtures of these solvents may be used; the preferred solvent is diethyl ether. Suitable bases for use in the process when L is a hydrogen atom in compound of formula VI include, but are not limited to, metal hydrides such as sodium or potassium hydride, metal alkoxides such as sodium or potassium alkoxides, alkali metal hydroxides such as sodium or potassium hydroxide, tertiary amines such as triethylamine or diisopropylethylamine, an alkali metal carbonate such as sodium or potassium carbonate, or pyridine and the like. If desired, mixtures of these bases may be used; the preferred base is sodium hydride. The process is carried out at temperatures from about −78° C. to about 100° C., preferably from about 0° C. to about 50° C. The process is preferably carried out at about atmospheric pressure although higher or lower pressures can be used if desired. Substantially equimolar amounts of reactants are used although higher or lower amounts can be used if desired. Preferably, from about 0.9 to about 1.1 equivalents of a compound of formula (VI) are used per equivalent of a compound of formula (V). Preferably, about one equivalent of base is used per equivalent of a compound of formula (VI) when L is a hydrogen atom.

A third embodiment of this invention relates to a process leading to a compound of formula (II)

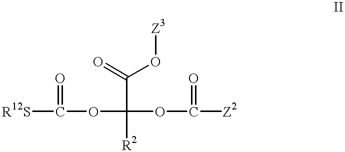

comprising the step of reacting, in the presence of a suitable solvent and a suitable base, a compound of formula (III)

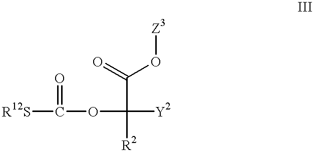

with a compound of formula (IV)

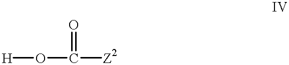

wherein

Z³ is alkyl, alkylcarbonyloxyalkyl, hydroxyalkyl, alkylsulfonylalkyl, acetylaminoalkyl, haloalkyl, alkenyl, acetylaminoalkenyl, haloalkenyl, alkynyl, haloalkynyl, cycloalkyl, cycloalkenyl, carboxycycloalkyl, carboxycycloalkenyl, cycloalkylalkyl, cycloalkylalkenyl, cycloalkenylalkyl, cycloalkenylalkenyl, cycloalkylalkynyl, cycloalkenylalkynyl, carboxycycloalkylalkyl, carboxycycloalkylalkenyl , carboxycycloalkenylalkyl, carboxycycloalkenylalkenyl, carboxycycloalkylalkynyl, carboxycycloalkenylalkynyl, heterocyclyl, heterocyclylalkyl, heterocyclylalkenyl, heterocyclylalkynyl, alkoxyalkyl, alkoxyalkoxyalkyl, alkoxyalkenyl, alkoxyalkynyl, alkoxycarbonylalkyl, alkoxycarbonylalkenyl, alkoxycarbonylalkynyl, haloalkoxyalkyl, haloalkoxyalkenyl, haloalkoxyalkynyl, alkylthioalkyl, alkylthlioalkenyl, alkylthioalkynyl, haloalkylthioalkyl, haloalkylthioalkenyl, haloalkylthioalkynyl, carboxyalkyl, carboxyalkenyl, carboxyalkynyl, SiR³R⁴R⁵, aryl, aryl substituted with one or more substituents independently selected from halo, nitro, hydroxy, cyano, thiocyanato, alkyl, alkylsulfonylalkyl, alkenyl, alkynyl, haloalkyl, haloalkenyl, haloalkynyl, alkoxy, haloalkoxy, SO₂NR³R⁴ and NR³R⁴, aralkyl, aralkenyl, aralkynyl, arcycloalkyl, aroxyalkyl, or aralkyl, aralkenyl, aralkynyl, arcycloalkyl, aroxyalkyl substituted with one or more substituents independently selected from halo, nitro, hydroxy, cyano, alkyl, cycloalkyl, alkenyl, alkynyl, haloalkyl, haloalkenyl, haloalkynyl, alkoxy, haloalkoxy, SO₂NR³R⁴ and NR³R⁴, heteroaryl, heteroaryl substituted with one or more substituents independently selected from halo, nitro, hydroxy, cyano, alkyl, alkenyl, alkynyl, haloalkyl, haloalkenyl, haloalkynyl, alkoxy, haloalkoxy, SO₂NR³R⁴ and NR³R⁴, heteroaralkyl, heteroaralkenyl, heteroaralkynyl, or heteroaralkyl, heteroaralkenyl, heteroaralkynyl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴,

Z² is a hydrogen atom, alkyl, alkylcarbonyloxyalkyl, alkylcarbonyl, alkenylcarbonyl, alkynylcarbonyl, hydroxyalkyl, alkylsulfonylalkyl, acetylaminoalkyl, haloalkyl, alkenyl, acetylaminoalkenyl, haloalkenyl, alkynyl, haloalkynyl, cycloalkyl, cycloalkenyl, carboxycycloalkyl, carboxycycloalkenyl, cycloalkylalkyl, cycloalkylalkenyl, cycloalkenylalkyl, cycloalkenylalkenyl, cycloalkylalkynyl, cycloalkenylalkynyl, carboxycycloalkylalkyl, carboxycycloalkylalkenyl, carboxycycloalkenylalkyl, carboxycycloalkenylalkenyl, carboxycycloalkylalkynyl, carboxycycloalkenylalkynyl, heterocyclyl, heterocyclylalkyl, heterocyclylalkenyl, heterocyclylalkynyl, alkoxyalkyl, alkoxyalkoxyalkyl, alkoxyalkenyl, alkoxyalkynyl, alkoxycarbonylalkyl, alkoxycarbonylalkenyl, alkoxycarbonylalkynyl, haloalkoxyalkyl, haloalkoxyalkenyl, haloalkoxyalkynyl, alkylthioalkyl, alkylthioalkenyl, alkylthioalkynyl, haloalkylthioalkyl, haloalkylthioalkenyl, haloalkylthioalkynyl, NR³R⁴, SO₂NR³R⁴, carboxyalkyl, carboxyalkenyl, carboxyalkynyl, dialkoxyphosphorylalkyl, aryl, aryl substituted with one or more substituents independently selected from halo, nitro, hydroxy, cyano, thiocyanato, alkyl, alkylsulfonylalkyl, alkenyl, alkynyl, haloalkyl, haloalkenyl, haloalkynyl, alkoxy, haloalkoxy, SO₂NR³R⁴ and NR³R⁴, aralkyl, aralkenyl, aralkynyl, arcycloalkyl, aroxyalkyl, or aralkyl, aralkenyl, aralkynyl, arcycloalkyl, aroxyalkyl substituted with one or more substituents independently selected from halo, nitro, hydroxy, cyano, alkyl, cycloalkyl, alkenyl, alkynyl, haloalkyl, haloalkenyl, haloalkynyl, alkoxy, haloalkoxy, SO₂NR³R⁴ and NR³R⁴, heteroaryl, heteroaryl substituted with one or more substituents independently selected from halo, nitro, hydroxy, cyano, alkyl, alkenyl, alkynyl, haloalkyl, haloalkenyl, haloalkynyl, alkoxy, haloalkoxy, SO₂NR³R⁴ and NR³R⁴, heteroaralkyl, heteroaralkenyl, heteroaralkynyl, or heteroaralkyl, heteroaralkenyl, heteroaralkynyl substituted with one or more substituents independently selected from halo, hydroxy, nitro, cyano, alkyl, alkenyl, alkynyl, haloalkyl, haloalkenyl, haloalkynyl, alkoxy, haloalkoxy, SO₂NR³R⁴ and NR³R⁴, alkylcarbonylalkyl, alkenylcarbonylalkyl, alkynylcarbonylalkyl, heterocyclylcarbonyl, heterocyclylcarbonylalkyl, heterocyclyloxycarbonylalkyl, arylcarbonyl, arylcarbonylalkyl, aralkylcarbonyl, aralkylcarbonylalkyl, aroxycarbonylalkyl, aralkoxycarbonylalkyl, heteroarylcarbonyl, heteroarylcarbonylalkyl, heteroaroxycarbonylalkyl, or arylcarbonyl, arylcarbonylalkyl, aralkylcarbonyl, aralkylcarbonylalkyl, aroxycarbonylalkyl, aralkoxycarbonylalkyl, heteroarylcarbonyl, heteroarylcarbonylalkyl, heteroaroxycarbonylalkyl substituted with one or more substituents independently selected from halo, hydroxy, nitro, cyano, alkyl, alkenyl, alkynyl, haloalkyl, haloalkenyl, haloalkynyl, alkoxy, haloalkoxy, SO₂NR³R⁴ and NR³R⁴, and C(═N—G²²)R²,

G²² is OR³, OCOR³, S(O)ⱼR³, OS(O)ⱼR³, NR³R⁴, OSO₂NR³R⁴, OP(═O)OR³NR³R⁴, OP(═O)(OR³)₂ or N═CR³R⁴,

j is 0, 1 or 2,

R² is a hydrogen atom, alkyl, alkenyl, alkynyl, alkoxyalkyl, alkoxyalkenyl, alkoxyalkynyl, alkylthioalkyl, alkylthioalkenyl, alkylthioalkynyl, carboxy, a carboxylate salt, carboxyalkyl, carboxyalkenyl, carboxyalkynyl, alkoxycarbonyl, alkoxycarbonylalkyl, alkoxycarbonylalkenyl, alkoxycarbonylalkynyl, alkylcarbonyl, alkenylcarbonyl, alkynylcarbonyl, cycloalkyl, cycloalkenyl, cycloalkylalkyl, cycloalkenylalkyl, cycloalkylalkenyl, cycloalkenylalkenyl, cycloalkylalkynyl, cycloalkenylalkynyl, heterocyclyl, heterocyclylalkyl, heterocyclylalkenyl, heterocyclylalkynyl, or alkyl, alkenyl, alkynyl, alkoxyalkyl, alkoxyalkenyl, alkoxyalkynyl, alkylthioalkyl, alkylthioalkenyl, alkylthioalkynyl, carboxyalkyl, carboxyalkenyl, carboxyalkynyl, alkoxycarbonyl, alkoxycarbonylalkyl, alkoxycarbonylalkenyl, alkoxycarbonylalkynyl, alkylcarbonyl, alkenylcarbonyl, alkynylcarbonyl, cycloalkyl, cycloalkenyl, cycloalkylalkyl, cycloalkenylalkyl, cycloalkylalkenyl, cycloalkenylalkenyl, cycloalkylalkynyl, cycloalkenylalkynyl, heterocyclyl, heterocyclylalkyl, heterocyclylalkenyl, heterocyclylalkynyl substituted with one or more substituents independently selected from halo, cyano, hydroxy, nitro, SO₂NR³R⁴ and NR³R⁴, aryl or aryl substituted with one or more substituents independently selected from halo, hydroxy, cyano, nitro, alkyl, alkenyl, alkynyl, haloalkyl, haloalkenyl, haloalkynyl, alkoxy, haloalkoxy, carboxy, alkoxycarbonyl, SO₂NR³R⁴ and NR³R⁴, aralkyl, aralkenyl, aralkynyl or aralkyl, aralkenyl, aralkynyl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, arylcarbonyl, aralkylcarbonyl, aralkenylcarbonyl, aralkynylcarbonyl or arylcarbonyl, aralkylcarbonyl, aralkenylcarbonyl, aralkynylcarbonyl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, heteroaryl or heteroaryl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, heteroaralkyl, heteroaralkenyl, heteroaralkynyl or heteroaralkyl, heteroaralkenyl, heteroaralkynyl substituted with one or more substituents independently selected from halo, cyano, hydroxy, nitro, alkyl, alkenyl, alkynyl, haloalkyl, haloalkenyl, haloalkynyl, alkoxy, haloalkoxy, SO₂NR³R⁴ and NR³R⁴, heteroaralkylcarbonyl, heteroaralkenylcarbonyl, heteroaralkynylcarbonyl or heteroaralkylcarbonyl, heteroaralkenylcarbonyl, heteroaralkynylcarbonyl substituted with one or more substituents independently selected from halo, cyano, hydroxy, nitro, alkyl, alkenyl, alkynyl, haloalkyl, haloalkenyl, haloalkynyl, alkoxy, haloalkoxy, SO₂NR³R⁴ and NR³R⁴,

Y² is halo,

R¹² is alkyl, cycloalkyl, cycloalkenyl, cycloalkylalkyl, cycloalkylalkenyl, cycloalkylalkynyl, cycloalkenylalkyl, cycloalkenylalkenyl, cycloalkenylalkynyl, carboxyalkyl, carboxyalkenyl, carboxyalkynyl, heterocyclyl, heterocyclylalkyl, heterocyclylalkenyl, heterocyclylalkynyl, alkoxyalkyl, alkenyl, alkynyl, or alkyl, cycloalkyl, cycloalkenyl, cycloalkylalkyl, cycloalkylalkenyl, cycloalkylalkynyl, cycloalkenylalkyl, cycloalkenylalkenyl, cycloalkenylalkynyl, carboxyalkyl, carboxyalkenyl, carboxyalkynyl, heterocyclyl, heterocyclylalkyl, heterocyclylalkenyl, heterocyclylalkynyl, alkoxyalkyl, alkenyl or alkynyl substituted with one or more halo, aryl or aryl substituted with one or more substituents independently selected from halo, nitro, cyano, haloalkyl, haloalkenyl, haloalkynyl, alkylcarbonyl, alkenylcarbonyl, alkynylcarbonyl, arylcarbonyl, alkylsulfonyl and arylsulfonyl, aralkyl, aralkenyl, aralkynyl, or aralkyl, aralkenyl, aralkynyl substituted with one or more substituents independently selected from halo, alkyl, alkenyl, alkynyl, haloalkyl, haloalkenyl, haloalkynyl, alkoxy and haloalkoxy, heteroaryl, heteroaralkyl, heteroaralkenyl, heteroaralkynyl, or heteroaryl, heteroaralkyl, heteroaralkenyl, heteroaralkynyl substituted with one or more substituents independently selected from halo, alkyl, alkenyl, alkynyl, haloalkyl, haloalkenyl, haloalkynyl, alkoxy and haloalkoxy,

R³, R⁴ and R⁵ are each independently a hydrogen atom, alkyl, cycloalkyl, cycloalkenyl, cycloalkylalkyl, cycloalkylalkenyl, cycloalkylalkynyl, cycloalkenylalkyl, cycloalkenylalkenyl, cycloalkenylalkynyl, carboxyalkyl, carboxyalkenyl, carboxyalkynyl, heterocyclyl, heterocyclylalkyl, heterocyclylalkenyl, heterocyclylalkynyl, alkoxyalkyl, alkenyl, alkynyl, or alkyl, cycloalkyl, cycloalkenyl, cycloalkylalkyl, cycloalkylalkenyl, cycloalkylalkynyl, cycloalkenylalkyl, cycloalkenylalkenyl, cycloalkenylalkynyl, carboxyalkyl, carboxyalkenyl, carboxyalkynyl, heterocyclyl, heterocyclylalkyl, heterocyclylalkenyl, heterocyclylalkynyl, alkoxyalkyl, alkenyl or alkynyl substituted with one or more halo, aryl, aralkyl, aralkenyl, aralkynyl, or aryl, aralkyl, aralkenyl, aralkynyl substituted with one or more substituents independently selected from halo, alkyl, alkenyl, alkynyl, haloalkyl, haloalkenyl, haloalkynyl, alkoxy and haloalkoxy, heteroaryl, heteroaralkyl, heteroaralkenyl, heteroaralkynyl, or heteroaryl, heteroaralkyl, heteroaralkenyl, heteroaralkynyl substituted with one or more substituents independently selected from halo, alkyl, alkenyl, alkynyl, haloalkyl, haloalkenyl, haloalkynyl, alkoxy and haloalkoxy, or R³ and R⁴ taken together with the nitrogen atom to which they are attached form a 5-or 6-membered saturated or unsaturated heterocyclic ring.

In a preferred mode of this third embodiment,

Y² is chloro, bromo or iodo,

R¹² is alkyl, aralkyl, aryl, heteroaryl, or aralkyl, aryl or heteroaryl substituted with one or more substituents independently selected from halo, alkyl, alkenyl, alkynyl, haloalkyl, haloalkenyl, haloalkynyl, alkoxy and haloalkoxy,

Z³ is alkyl, aryl, aralkyl or aryl or aralkyl substituted with one or more substituents independently selected from halo, alkyl, alkenyl, alkynyl, haloalkyl, haloalkenyl, haloalkynyl, alkoxy and haloalkoxy, and

R² is a hydrogen atom.

In a more preferred mode of this third embodiment, Y² is chloro or iodo, R¹² is alkyl and Z³ is alkyl or aralkyl.

In another more preferred mode of this third embodiment,

Z³ is (C₁-C₂₀)alkyl, (C₁-C₁₀)alkylcarbonyloxy(C₁-C₁₀)alkyl, hydroxy(C₁-C₂₀) alkyl, (C₁-C₁₀)alkylsulfonyl(C₁-C₁₀)alkyl, acetylamino(C₁-C₁₀)alkyl, halo(C₁-C₂₀) alkyl, (C₂-C₁₀)alkenyl, acetylamino(C₂-C₁₀)alkenyl, halo(C₂-C₁₀)alkenyl, (C₂-C₁₀) alkynyl, halo(C₂-C₁₀)alkynyl, cyclo(C₃-C₈)alkyl, cyclo(C₃-C₈)alkenyl, carboxycyclo(C₃-C₈)alkyl, carboxycyclo(C₃-C₈)alkenyl, cyclo(C₃-C₈)alkyl(C₁-C₁₀) alkyl, cyclo(C₃-C₈)alkyl(C₂-C₁₀)alkenyl, cyclo(C₃-C₈)alkenyl(C₁-C₁₀)alkyl, cyclo(C₃-C₈)alkenyl(C₂-C₁₀)alkenyl, cyclo(C₃-C₈)alkyl(C₂-C₁₀)alkynyl, cyclo(C₃-C₈) alkenyl(C₂-C₁₀)alkynyl, carboxycyclo(C₃-C₈)alkyl(C₁-C₁₀)alkyl, carboxycyclo(C₃-C₈)alkyl(C₂-C₁₀)alkenyl, carboxycyclo(C₃-C₈)alkenyl(C₁-C₁₀)alkyl, carboxycyclo(C₃-C₈)alkenyl(C₂-C₁₀)alkenyl, carboxycyclo(C₃-C₈)alkyl(C₂-C₁₀) alkynyl, carboxycyclo(C₃-C₈)alkenyl(C₂-C₁₀)alkynyl, heterocyclyl, heterocyclyl(C₁-C₁₀)alkyl, heterocyclyl(C₂-C₁₀)alkenyl, heterocyclyl(C₂-C₁₀) alkynyl, (C₁-C₁₀)alkoxy(C₁-C₁₀)alkyl, (C₁-C₁₀)alkoxy(C₁-C₁₀)alkoxy(C₁-C₁₀) alkyl, (C₁-C₁₀)alkoxy(C₂-C₁₀)alkenyl, (C₁-C₁₀)alkoxy(C₂-C₁₀)alkynyl, (C₁-C₁₀) alkoxycarbonyl(C₁-C₁₀)alkyl, (C₁-C₁₀)alkoxycarbonyl(C₂-C₁₀)alkenyl, (C₁-C₁₀) alkoxycarbonyl(C₂-C₁₀)alkynyl, halo(C₁-C₁₀)alkoxy(C₁-C₁₀)alkyl, halo(C₁-C₁₀) alkoxy(C₂-C₁₀)alkenyl, halo(C₁-C₁₀)alkoxy(C₂-C₁₀)alkynyl, (C₁-C₁₀)alkylthio(C₁-C₁₀) alkyl, (C₁-C₁₀)alkylthio(C₂-C₁₀)alkenyl, (C₁-C₁₀)alkylthio(C₂-C₁₀) alkynyl, halo(C₁-C₁₀)alkylthio(C₁-C₁₀)alkyl, halo(C₁-C₁₀)alkylthio(C₂-C₁₀)alkenyl, halo (C₁-C₁₀)alkylthio(C₂-C₁₀)alkynyl, carboxy(C₁-C₂₀)alkyl, carboxy(C₂-C₁₀)alkenyl, carboxy(C₂-C₁₀)alkynyl, SiR³R⁴R⁵, aryl, aryl substituted with one or more substituents independently selected from halo, nitro, hydroxy, cyano, thiocyanato, (C₁-C₁₀)alkyl, (C₁-C₁₀)alkylsulfonyl(C₁-C₁₀)alkyl, (C₂-C₁₀) alkenyl, (C₂-C₁₀)alkynyl, halo(C₁-C₁₀)alkyl, halo(C₂-C₁₀)alkenyl, halo(C₂-C₁₀) alkynyl, (C₁-C₁₀)alkoxy, halo(C₁-C₁₀)alkoxy, SO₂NR³R⁴ and NR³R⁴, ar(C₁-C₁₀) alkyl, ar(C₂-C₁₀)alkenyl, ar(C₂-C₁₀)alkynyl, arcyclo(C₃-C₈)alkyl, aroxy(C₁-C₁₀) alkyl, or ar(C₁-C₁₀)alkyl, ar(C₂-C₁₀)alkenyl, ar(C₂-C₁₀)alkynyl, arcyclo(C₃-C₈) alkyl, aroxy(C₁-C₁₀)alkyl substituted with one or more substituents independently selected from halo, nitro, hydroxy, cyano, (C₁-C₁₀)alkyl, cycloalkyl, (C₂-C₁₀)alkenyl, (C₂-C₁₀)alkynyl, halo(C₁-C₁₀)alkyl, halo(C₂-C₁₀)alkenyl, halo(C₂-C₁₀) alkynyl, (C₁-C₁₀)alkoxy, halo(C₁-C₁₀)alkoxy, SO₂NR³R⁴ and NR³R⁴, heteroaryl, heteroaryl substituted with one or more substituents independently selected from halo, nitro, hydroxy, cyano, (C₁-C₁₀)alkyl, (C₂-C₁₀)alkenyl, (C₂-C₁₀) alkynyl, halo(C₁-C₁₀)alkyl, halo(C₂-C₁₀)alkenyl, halo(C₂-C₁₀)alkynyl, (C₁-C₁₀) alkoxy, halo(C₁-C₁₀)alkoxy, SO₂NR³R⁴ and NR³R⁴, heteroar(C₁-C₁₀)alkyl, heteroar(C₂-C₁₀)alkenyl, heteroar(C₂-C₁₀)alkynyl, or heteroar(C₁-C₁₀)alkyl, heteroar(C₂-C₁₀)alkenyl, heteroar(C₂-C₁₀)alkynyl substituted with one or more substituents independently selected from halo, hydroxy, cyano, nitro, (C₁-C₁₀) alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴,

Z² is a hydrogen atom, (C₁-C₂₀)alkyl, (C₁-C₁₀)alkylcarbonyloxy(C₁-C₁₀) alkyl, (C₁-C₂₀)alkylcarbonyl, (C₁-C₂₀)alkenylcarbonyl, (C₁-C₂₀) alkynylcarbonyl, hydroxy(C₁-C₂₀)alkyl, (C₁-C₁₀)alkylsulfonyl(C₁-C₁₀)alkyl, acetylamino(C₁-C₁₀)alkyl, halo(C₁-C₂₀)alkyl, (C₂-C₂₀)alkenyl, halo(C₂-C₂₀)alkenyl, acetylamino(C₂-C₁₀)alkenyl, (C₂-C₂₀)alkynyl, halo(C₂-C₂₀)alkynyl, cyclo(C₃-C₈) alkyl, cyclo(C₃-C₈)alkenyl, carboxycyclo(C₃-C₈)alkyl, carboxycyclo(C₃-C₈) alkenyl, cyclo(C₃-C₈)alkyl(C₁-C₁₀)alkyl, cyclo(C₃-C₈)alkyl(C₂-C₁₀)alkenyl, cyclo(C₃-C₈)alkenyl(C₁-C₁₀)alkyl, cyclo(C₃-C₈)alkenyl(C₂-C₁₀)alkenyl, cyclo(C₃-C₈) alkyl(C₂-C₁₀)alkynyl, cyclo(C₃-C₈)alkenyl(C₂-C₁₀)alkynyl, carboxycyclo(C₃-C₈) alkyl(C₁-C₁₀)alkyl, carboxy(C₃-C₈)cycloalkyl(C₂-C₁₀)alkenyl, carboxycyclo(C₃-C₈) alkenyl(C₁-C₁₀)alkyl, carboxycyclo(C₃-C₈)alkenyl(C₂-C₁₀)alkenyl, carboxycyclo(C₃-C₈)alkyl(C₂-C₁₀)alkynyl, carboxycyclo(C₃-C₈)alkenyl(C₂-C₁₀) alkynyl, heterocyclyl, heterocyclyl(C₁-C₁₀)alkyl, heterocyclyl(C₂-C₁₀)alkenyl, heterocyclyl(C₂-C₁₀)alkynyl, (C₁-C₁₀)alkoxy(C₁-C₁₀)alkyl, (C₁-C₅)alkoxy(C₁-C₅) alkoxy(C₁-C₁₀)alkyl, (C₁-C₁₀)alkoxy(C₂-C₁₀)alkenyl, (C₁-C₁₀)alkoxy(C₂-C₁₀) alkynyl, (C₁-C₁₀)alkoxycarbonyl(C₁-C₁₀)alkyl, (C₁-C₁₀)alkoxycarbonyl(C₂-C₁₀) alkenyl, (C₁-C₁₀)alkoxycarbonyl(C₂-C₁₀)alkynyl, halo(C₁-C₁₀)alkoxy(C₁-C₁₀) alkyl, halo(C₁-C₁₀)alkoxy(C₂-C₁₀)alkenyl, halo(C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀) alkylthio(C₂-C₁₀)alkynyl, halo(C₁-C₁₀)alkylthio(C₁-C₁₀)alkyl, halo(C₁-C₁₀) alkylthio(C₂-C₁₀)alkenyl, halo(C₁-C₁₀)alkylthio(C₂-C₁₀)alkynyl, SO₂NR³R⁴, NR³R⁴, carboxy(C₁-C₂₀)alkyl, carboxy(C₂-C₂₀)alkenyl, carboxy(C₂-C₂₀)alkynyl, di(C₁-C₁₀)alkoxyphosphoryl(C₁-C₁₀)alkyl, aryl, aryl substituted with one or more substituents independently selected from halo, nitro, cyano, hydroxy, (C₁-C₁₀) alkyl, (C₁-C₁₀)alkylsulfonyl(C₁-C₁₀)alkyl, (C₁-C₁₀)alkylsulfonyl, thiocyanato, (C₂-C₁₀)alkenyl, (C₂-C₁₀)alkynyl, halo(C₁-C₁₀)alkyl, halo(C₂-C₁₀)alkenyl, halo(C₂-C₁₀) alkynyl, (C₁-C₁₀)alkoxy, halo(C₁-C₁₀)alkoxy, SO₂NR³R⁴, and NR³R⁴, ar(C₁-C₁₀) alkyl, ar(C₂-C₁₀)alkenyl, ar(C₂-C₁₀)alkynyl, arcyclo(C₃-C₈)alkyl, aroxy(C₁-C₁₀) alkyl, or ar(C₁-C₁₀)alkyl, ar(C₂-C₁₀)alkenyl, ar(C₂-C₁₀)alkynyl, arcyclo(C₃-C₈) alkyl, aroxy(C₁-C₁₀)alkyl substituted with one or more substituents independently selected from halo, nitro, hydroxy, cyano, (C₁-C₁₀)alkyl, cyclo(C₃-C₈) alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroaryl, heteroaryl substituted with one or more substituents independently selected from halo, hydroxy, nitro, cyano, (C₁-C₁₀)alkyl, (C₂-C₁₀)alkenyl, (C₂-C₁₀) alkynyl, halo(C₁-C₁₀)alkyl, halo(C₂-C₁₀)alkenyl, halo(C₂-C₁₀)alkynyl, (C₁-C₁₀) alkoxy, halo(C₁-C₁₀)alkoxy and NR³R⁴, heteroar(C₁-C₁₀)alkyl, heteroar(C₂-C₁₀) alkenyl, heteroar(C₂-C₁₀)alkynyl, or heteroar(C₁-C₁₀)alkyl, heteroar(C₂-C₁₀) alkenyl, heteroar(C₂-C₁₀)alkynyl substituted with one or more substituents independently selected from halo, hydroxy, cyano, nitro, (C₁-C₁₀)alkyl, (C₂-C₁₀) alkenyl, (C₂-C₁₀)alkynyl, halo(C₁-C₁₀)alkyl, halo(C₂-C₁₀)alkenyl, halo(C₂-C₁₀) alkynyl, (C₁-C₁₀)alkoxy, halo(C₁-C₁₀)alkoxy, SO₂NR³R⁴ and NR³R⁴, (C₁-C₁₀) alkylcarbonyl(C₁-C₁₀)alkyl, (C₂-C₁₀)alkenylcarbonyl(C₁-C₁₀)alkyl, (C₂-C₁₀) alkynylcarbonyl(C₁-C₁₀)alkyl, heterocyclylcarbonyl, heterocyclylcarbonyl(C₁-C₁₀) alkyl, heterocyclyloxycarbonyl(C₁-C₁₀)alkyl, arylcarbonyl, arylcarbonyl(C₁-C₁₀) alkyl, ar(C₁-C₁₀)alkylcarbonyl, ar(C₁-C₁₀)alkylcarbonyl(C₁-C₁₀)alkyl, aroxycarbonyl(C₁-C₁₀)alkyl, ar(C₁-C₁₀)alkoxycarbonyl(C₁-C₁₀)alkyl, heteroarylcarbonyl, heteroarylcarbonyl(C₁-C₁₀)alkyl, heteroaroxycarbonyl(C₁-C₁₀) alkyl, or arylcarbonyl, arylcarbonyl(C₁-C₁₀)alkyl, ar(C₁-C₁₀)alkylcarbonyl, ar(C₁-C₁₀)alkylcarbonyl(C₁-C₁₀)alkyl, aroxycarbonyl(C₁-C₁₀)alkyl, ar(C₁-C₁₀) alkoxycarbonyl(C₁-C₁₀)alkyl, heteroarylcarbonyl, heteroarylcarbonyl(C₁-C₁₀) alkyl, heteroaroxycarbonyl(C₁-C₁₀)alkyl substituted with one or more substituents independently selected from halo, hydroxy, nitro, cyano, (C₁-C₁₀) alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, and C(═N—G²²)R²,

G²² is OR³, OCOR³, S(O)ⱼR³, OS(O)ⱼR³, NR³R⁴, OSO₂NR³R⁴, OP(═O)OR³NR³R⁴, OP(═O)(OR³)₂ or N═CR³R⁴,

j is 0, 1 or 2,

R² is a hydrogen atom, (C₁-C₂₀)alkyl, (C₂-C₁₀)alkenyl, (C₂-C₁₀)alkynyl, (C₁-C₁₀) alkoxy(C₁-C₁₀)alkyl, (C₁-C₁₀)alkoxy(C₂-C₁₀)alkenyl, (C₁-C₁₀)alkoxy(C₂-C₁₀) alkynyl, (C₁-C₁₀)alkylthio(C₁-C₁₀)alkyl, (C₁-C₁₀)alkylthio(C₂-C₁₀)alkenyl, (C₁-C₁₀) alkylthio(C₂-C₁₀)alkynyl, carboxy, a carboxylate salt, carboxy(C₁-C₂₀)alkyl, carboxy(C₂-C₂₀)alkenyl, carboxy(C₂-C₂₀)alkynyl, (C₁-C₂₀)alkoxycarbonyl, (C₁-C₁₀) alkoxycarbonyl(C₁-C₁₀)alkyl, (C₁-C₁₀)alkoxycarbonyl(C₂-C₁₀)alkenyl, (C₁-C₁₀) alkoxycarbonyl(C₂-C₁₀)alkynyl, (C₁-C₂₀)alkylcarbonyl, (C₂-C₂₀) alkenylcarbonyl, (C₂-C₂₀)alkynylcarbonyl, cyclo(C₃-C₈)alkyl, cyclo(C₃-C₈) alkenyl, cyclo(C₃-C₈)alkyl(C₁-C₁₀)alkyl, cyclo(C₃-C₈)alkenyl(C₁-C₁₀)alkyl, cyclo(C₃-C₈)alkyl(C₂-C₁₀)alkenyl, cyclo(C₃-C₈)alkenyl(C₂-C₁₀)alkenyl, cyclo(C₃-C₈) alkyl(C₂-C₁₀)alkynyl, cyclo(C₃-C₈)alkenyl(C₂-C₁₀)alkynyl, heterocyclyl, heterocyclyl(C₁-C₁₀)alkyl, heterocyclyl(C₂-C₁₀)alkenyl, heterocyclyl(C₂-C₁₀) alkynyl, or (C₁-C₂₀)alkyl, (C₂-C₁₀)alkenyl, (C₂-C₁₀)alkynyl, (C₁-C₁₀)alkoxy(C₁-C₁₀) alkyl, (C₁-C₁₀)alkoxy(C₂-C₁₀)alkenyl, (C₁-C₁₀)alkoxy(C₂-C₁₀)alkynyl, (C₁-C₁₀) alkylthio(C₁-C₁₀)alkyl, (C₁-C₁₀)alkylthio(C₂-C₁₀)alkenyl, (C₁-C₁₀)alkylthio(C₂-C₁₀) alkynyl, carboxy(C₁-C₂₀)alkyl, carboxy(C₂-C₁₀)alkenyl, carboxy(C₂-C₁₀) alkynyl, (C₁-C₂₀)alkoxycarbonyl, (C₁-C₁₀)alkoxycarbonyl(C₁-C₁₀)alkyl, (C₁-C₁₀) alkoxycarbonyl(C₂-C₁₀)alkenyl, (C₁-C₁₀)alkoxycarbonyl(C₂-C₁₀)alkynyl, (C₁-C₂₀) alkylcarbonyl, (C₂-C₁₀)alkenylcarbonyl, (C₂-C₁₀)alkynylcarbonyl, cyclo(C₃-C₈) alkyl, cyclo(C₃-C₈)alkenyl, cyclo(C₃-C₈)alkyl(C₁-C₁₀)alkyl, cyclo(C₃-C₈) alkenyl(C₁-C₁₀)alkyl, cyclo(C₃-C₈)alkyl(C₂-C₁₀)alkenyl, cyclo(C₃-C₈)alkenyl(C₂-C₁₀) alkenyl, cyclo(C₃-C₈)alkyl(C₂-C₁₀)alkynyl, cyclo(C₃-C₈)alkenyl(C₂-C₁₀)alkynyl, heterocyclyl, heterocyclyl(C₁-C₁₀)alkyl, heterocyclyl(C₂-C₁₀)alkenyl, heterocyclyl(C₂-C₁₀)alkynyl substituted with one or more substituents independently selected from halo, cyano, hydroxy, nitro, SO₂NR³R⁴ and NR³R⁴, aryl or aryl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, carboxy, (C₁-C₄)alkoxycarbonyl, SO₂NR³R⁴ and NR³R⁴, ar(C₁-C₁₀)alkyl, ar(C₂-C₁₀) alkenyl, ar(C₂-C₁₀)alkynyl, or ar(C₁-C₁₀)alkyl, ar(C₂-C₁₀)alkenyl, ar(C₂-C₁₀) alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, arylcarbonyl, ar(C₁-C₁₀)alkylcarbonyl, ar(C₂-C₁₀) alkenylcarbonyl, ar(C₂-C₁₀)alkynylcarbonyl or arylcarbonyl, ar(C₁-C₁₀) alkylcarbonyl, ar(C₂-C₁₀)alkenylcarbonyl, ar(C₂-C₁₀)alkynylcarbonyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀) alkyl, halo(C₂-C₁₀)alkenyl, halo(C₂-C₁₀)alkynyl, (C₁-C₁₀)alkoxy, halo(C₁-C₁₀) alkoxy, SO₂NR³R⁴ and NR³R⁴, heteroaryl, heteroaryl substituted with one or more substituents independently selected from halo, (C₁-C₁₀)alkyl, (C₂-C₁₀) alkenyl, (C₂-C₁₀)alkynyl, halo(C₁-C₁₀)alkyl, halo(C₂-C₁₀)alkenyl, halo(C₂-C₁₀)alkynyl, (C₁-C₁₀) alkoxy, halo(C₁-C₁₀)alkoxy and NR³R⁴, heteroar(C₁-C₁₀)alkyl, heteroar(C₂-C₁₀)alkenyl, heteroar(C₂-C₁₀)alkynyl or heteroar(C₁-C₁₀)alkyl, heteroar(C₂-C₁₀)alkenyl, heteroar(C₂-C₁₀)alkynyl substituted with one or more substituents independently selected from halo, (C₁-C₁₀)alkyl, (C₂-C₁₀)alkenyl, (C₂-C₁₀) alkynyl, halo(C₁-C₁₀)alkyl, halo(C₂-C₁₀)alkenyl, halo(C₂-C₁₀)alkynyl, (C₁-C₁₀) alkoxy, halo(C₁-C₁₀)alkoxy, SO₂NR³R⁴ and NR³R⁴, heteroar(C₁-C₁₀) alkylcarbonyl, heteroar(C₂-C₁₀)alkenylcarbonyl, heteroar(C₂-C₁₀) alkynylcarbonyl or heteroar(C₁-C₁₀)alkylcarbonyl, heteroar(C₂-C₁₀) alkenylcarbonyl, heteroar(C₂-C₁₀)alkynylcarbonyl substituted with one or more substituents independently selected from halo, cyano, hydroxy, nitro, (C₁-C₁₀) alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, or R¹ and R² taken together with the carbon atom to which they are attached form a 5-7 membered saturated or unsaturated ring,

R³, R⁴ and R⁵ are each independently a hydrogen atom, (C₁-C₂₀)alkyl, cyclo(C₃-C₈)alkyl, cyclo(C₃-C₈)alkenyl, cyclo(C₃-C₈)alkyl(C₁-C₁₀)alkyl, cyclo(C₃-C₈) alkyl(C₂-C₁₀)alkenyl, cyclo(C₃-C₈)alkyl(C₂-C₁₀)alkynyl, cyclo(C₃-C₈)alkenyl(C₁-C₁₀) alkyl, cyclo(C₃-C₈)alkenyl(C₂-C₁₀)alkenyl, cyclo(C₃-C₈)alkenyl(C₂-C₁₀)alkynyl, carboxy(C₁-C₂₀)alkyl, carboxy(C₂-C₁₀)alkenyl, carboxy(C₂-C₁₀)alkynyl, heterocyclyl, heterocyclyl(C₁-C₁₀)alkyl, heterocyclyl(C₂-C₁₀)alkenyl, heterocyclyl(C₂-C₁₀)alkynyl, (C₁-C₁₀)alkoxy(C₁-C₁₀)alkyl, (C₂-C₁₀)alkenyl, (C₂-C₁₀) alkynyl, or (C₁-C₁₀)alkyl, cyclo(C₃-C₈)alkyl, cyclo(C₃-C₈)alkenyl, cyclo(C₃-C₈) alkyl(C₁-C₁₀)alkyl, cyclo(C₃-C₈)alkyl(C₂-C₁₀)alkenyl, cyclo(C₃-C₈)alkyl(C₂-C₁₀) alkynyl, cyclo(C₃-C₈)alkenyl(C₁-C₁₀)alkyl, cyclo(C₃-C₈)alkenyl(C₂-C₁₀)alkenyl, cyclo(C₃-C₈)alkenyl(C₂-C₁₀)alkynyl, carboxy(C 1-C₂₀)alkyl, carboxy(C₂-C₁₀)alkenyl, carboxy(C₂-C₁₀)alkynyl, heterocyclyl, heterocyclyl(C₁-C₁₀)alkyl, heterocyclyl(C₂-C₁₀) alkenyl, heterocyclyl(C₂-C₁₀)alkynyl, (C₁-C₁₀)alkoxy(C₁-C₁₀)alkyl, (C₂-C₁₀) alkenyl or (C₂-C₁₀)alkynyl substituted with one or more halo, aryl, ar(C₁-C₁₀) alkyl, ar(C₂-C₁₀)alkenyl, ar(C₂-C₁₀)alkynyl or aryl, ar(C₁-C₁₀)alkyl, ar(C₂-C₁₀) alkenyl, ar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy and halo(C₁-C₁₀)alkoxy, heteroaryl, heteroar(C₁-C₁₀)alkyl, heteroar(C₂-C₁₀)alkenyl, heteroar(C₂-C₁₀)alkynyl or heteroaryl, heteroar(C₁-C₁₀)alkyl, heteroar(C₂-C₁₀) alkenyl, heteroar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy and halo(C₁-C₁₀)alkoxy, or R³ and R⁴ taken together with the nitrogen atom to which they are attached form a 5-or 6-membered saturated or unsaturated heterocyclic ring,

R¹² is (C₁-C₂₀)alkyl, cyclo(C₃-C₈)alkyl, cyclo(C₃-C₈)alkenyl, cyclo(C₃-C₈) alkyl(C₁-C₁₀)alkyl, cyclo(C₃-C₈)alkyl(C₂-C₁₀)alkenyl, cyclo(C₃-C₈)alkyl(C₂-C₁₀) alkynyl, cyclo(C₃-C₈)alkenyl(C₁-C₁₀)alkyl, cyclo(C₃-C₈)alkenyl(C₂-C₁₀)alkenyl, cyclo(C₃-C₈)alkenyl(C₂-C₁₀)alkynyl, carboxy(C₁-C₂₀)alkyl, carboxy(C₂-C₂₀)alkenyl, carboxy(C₂-C₂₀)alkynyl, heterocyclyl, heterocyclyl(C₁-C₁₀)alkyl, heterocyclyl(C₂-C₁₀) alkenyl, heterocyclyl(C₂-C₁₀)alkynyl, (C₁-C₁₀)alkoxy(C₁-C₁₀)alkyl, (C₂-C₂₀) alkenyl, (C₂-C₂₀)alkynyl, or (C₁-C₂₀)alkyl, cyclo(C₃-C₈)alkyl, cyclo(C₃-C₈) alkenyl, cyclo(C₃-C₈)alkyl(C₁-C₁₀)alkyl, cyclo(C₃-C₈)alkyl(C₂-C₁₀)alkenyl, cyclo(C₃-C₈)alkyl(C₂-C₁₀)alkynyl, cyclo(C₃-C₈)alkenyl(C₁-C₁₀)alkyl, cyclo(C₃-C₈) alkenyl(C₂-C₁₀)alkenyl, cyclo(C₃-C₈)alkenyl(C₂-C₁₀)alkynyl, carboxy(C₁-C₂₀) alkyl, carboxy(C₂-C₂₀)alkenyl, carboxy(C₂-C₂₀)alkynyl, heterocyclyl, heterocyclyl(C₁-C₁₀)alkyl, heterocyclyl(C₂-C₁₀)alkenyl, heterocyclyl(C₂-C₁₀) alkynyl, (C₁-C₁₀)alkoxy(C₁-C₁₀)alkyl, (C₂-C₂₀)alkenyl or (C₂-C₂₀)alkynyl substituted with one or more halo, aryl or aryl substituted with one or more substituents independently selected from halo, nitro, cyano, halo(C₁-C₁₀)alkyl, halo(C₂-C₁₀)alkenyl, halo(C₂-C₁₀)alkynyl, (C₁-C₁₀)alkylcarbonyl, (C₂-C₁₀) alkenylcarbonyl, (C₂-C₁₀)alkynylcarbonyl, arylcarbonyl, (C₁-C₁₀)alkylsulfonyl and arylsulfonyl, ar(C₁-C₁₀)alkyl, ar(C₂-C₁₀)alkenyl, ar(C₂-C₁₀)alkynyl, or ar(C₁-C₁₀) alkyl, ar(C₂-C₁₀)alkenyl, ar(C₂-C₁₀)alkynyl substituted with one or more substituents independently selected from halo, (C₁-C₁₀)alkyl, (C₂-C₁₀)alkenyl, (C₂-C₁₀) alkynyl, halo(C₁-C₁₀)alkyl, halo(C₂-C₁₀)alkenyl, halo(C₂-C₁₀)alkynyl, (C₁-C₁₀) alkoxy and halo(C₁-C₁₀)alkoxy, heteroaryl, heteroar(C₁-C₁₀)alkyl, heteroar(C₂-C₁₀)alkenyl, heteroar(C₂-C₁₀)alkynyl, or heteroaryl, heteroar(C₁-C₁₀) alkyl, heteroar(C₂-C₁₀)alkenyl, heteroar(C₂-C₁₀)alkynyl substituted with one or more substituents independently selected from halo, (C₁-C₁₀)alkyl, (C₂-C₁₀) alkenyl, (C₂-C₁₀)alkynyl, halo(C₁-C₁₀)alkyl, halo(C₂-C₁₀)alkenyl, halo(C₂-C₁₀) alkynyl, (C₁-C₁₀)alkoxy and halo(C₁-C₁₀)alkoxy, and

Y² is chloro, bromo or iodo.

In an even more preferred mode of this third embodiment,

Y² is chloro, bromo or iodo,

R¹² is (C₁-C₂₀)alkyl, ar(C₁-C₁₀)alkyl, aryl, heteroaryl or ar(C₁-C₁₀)alkyl, aryl or heteroaryl substituted with one or more substituents independently selected from halo, (C₁-C₁₀)alkyl, (C₂-C₁₀)alkenyl, (C₂-C₁₀)alkynyl, halo(C₁-C₁₀) alkyl, halo(C₂-C₁₀)alkenyl, halo(C₂-C₁₀)alkynyl, (C₁-C₁₀)alkoxy and halo(C₁-C₁₀) alkoxy,

Z³ is (C₁-C₂₀)alkyl, aryl, ar(C₁-C₁₀)alkyl or aryl or ar(C₁-C₁₀)alkyl substituted with one or more substituents independently selected from halo, (C₁-C₁₀) alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy and halo(C₁-C₁₀)alkoxy, and

R² is a hydrogen atom.

In yet an even more preferred mode of this third embodiment, Y² is chloro, or iodo, R¹² is (C₁-C₁₀)alkyl and Z³ is (C₁-C₁₀)alkyl or ar(C₁-C₆)alkyl.

In all of the modes of this third embodiment, suitable solvents for use in the process include, but are not limited to, ethers such as tetrahydrofuran (THF), glyme, diethyl ether and the like, esters such as ethyl acetate, alcohols, dimethylformamide (DMF), dimethylsulfoxide (DMSO), acetonitrile, chlorinated solvents such as methylene chloride (CH₂Cl₂) and chloroform (CHCl₃). If desired, mixtures of these solvents may be used. The preferred solvents are DMF and THF. Suitable bases for use in the above process include, but are not limited to, metal hydrides such as sodium or potassium hydride, metal alkoxides such as sodium or potassium alkoxides, alkali metal hydroxides such as sodium or potassium hydroxide, tertiary amines such as triethylamine or diisopropylethylamine, an alkali metal carbonate such as sodium or potassium carbonate, and pyridine. If desired, mixtures of these bases may be used. Preferred bases are triethylamine and diisopropylethylamine. The above process is carried out at temperatures from about −78° C. to about 100° C., preferably from about 0° C. to about 50° C. The process is preferably carried out at about atmospheric pressure although higher or lower pressures can be used if desired. From about 1 to about 10 equivalents of a compound of formula (IV) are used per equivalent of a compound of formula (III). Substantially equimolar amounts of reactants are preferably used. Generally, one equivalent of base is used per equivalent of a compound of formula (IV).

A fourth embodiment of this invention relates to a process leading to a compound of formula (I)

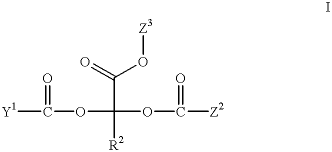

comprising the step of reacting, optionally in the presence of a suitable solvent, a compound of formula (II)

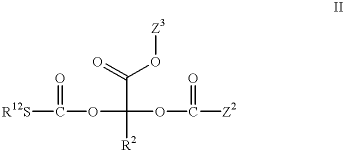

with a suitable halogenating agent wherein

Z³ is alkyl, alkylcarbonyloxyalkyl, hydroxyalkyl, alkylsulfonylalkyl, acetylaminoalkyl, haloalkyl, alkenyl, acetylaminoalkenyl, haloalkenyl, alkynyl, haloalkynyl, cycloalkyl, cycloalkenyl, carboxycycloalkyl, carboxycycloalkenyl, cycloalkylalkyl, cycloalkylalkenyl, cycloalkenylalkyl, cycloalkenylalkenyl, cycloalkylalkynyl, cycloalkenylalkynyl, carboxycycloalkylalkyl, carboxycycloalkylalkenyl, carboxycycloalkenylalkyl, carboxycycloalkenylalkenyl, carboxycycloalkylalkynyl, carboxycycloalkenylalkynyl, heterocyclyl, heterocyclylalkyl, heterocyclylalkenyl, heterocyclylalkynyl, alkoxyalkyl, alkoxyalkoxyalkyl, alkoxyalkenyl, alkoxyalkynyl, alkoxycarbonylalkyl, alkoxycarbonylalkenyl, alkoxycarbonylalkynyl, haloalkoxyalkyl, haloalkoxyalkenyl, haloalkoxyalkynyl, alkylthioalkyl, alkylthioalkenyl, alkylthioalkynyl, haloalkylthioalkyl, haloalkylthioalkenyl, haloalkylthioalkynyl, carboxyalkyl, carboxyalkenyl, carboxyalkynyl, SiR³R⁴R⁵, aryl, aryl substituted with one or more substituents independently selected from halo, nitro, hydroxy, cyano, thiocyanato, alkyl, alkylsulfonylalkyl, alkenyl, alkynyl, haloalkyl, haloalkenyl, haloalkynyl, alkoxy, haloalkoxy, SO₂NR³R⁴ and NR³R⁴, aralkyl, aralkenyl, aralkynyl, arcycloalkyl, aroxyalkyl, or aralkyl, aralkenyl, aralkynyl, arcycloalkyl, aroxyalkyl substituted with one or more substituents independently selected from halo, nitro, hydroxy, cyano, alkyl, cycloalkyl, alkenyl, alkynyl, haloalkyl, haloalkenyl, haloalkynyl, alkoxy, haloalkoxy, SO₂NR³R⁴ and NR³R⁴, heteroaryl, heteroaryl substituted with one or more substituents independently selected from halo, nitro, hydroxy, cyano, alkyl, alkenyl, alkynyl, haloalkyl, haloalkenyl, haloalkynyl, alkoxy, haloalkoxy, SO₂NR³R⁴ and NR³R⁴, heteroaralkyl, heteroaralkenyl, heteroaralkynyl, or heteroaralkyl, heteroaralkenyl, heteroaralkynyl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴,

Z² is a hydrogen atom, alkyl, alkylcarbonyloxyalkyl, alkylcarbonyl, alkenylcarbonyl, alkynylcarbonyl, hydroxyalkyl, alkylsulfonylalkyl, acetylaminoalkyl, haloalkyl, alkenyl, acetylaminoalkenyl, haloalkenyl, alkynyl, haloalkynyl, cycloalkyl, cycloalkenyl, carboxycycloalkyl, carboxycycloalkenyl, cycloalkylalkyl, cycloalkylalkenyl, cycloalkenylalkyl, cycloalkenylalkenyl, cycloalkylalkynyl, cycloalkenylalkynyl, carboxycycloalkylalkyl, carboxycycloalkylalkenyl, carboxycycloalkenylalkyl, carboxycycloalkenylalkenyl, carboxycycloalkylalkynyl, carboxycycloalkenylalkynyl, heterocyclyl, heterocyclylalkyl, heterocyclylalkenyl, heterocyclylalkynyl, alkoxyalkyl, alkoxyalkoxyalkyl, alkoxyalkenyl, alkoxyalkynyl, alkoxycarbonylalkyl, alkoxycarbonylalkenyl, alkoxycarbonylalkynyl, haloalkoxyalkyl, haloalkoxyalkenyl, haloalkoxyalkynyl, alkylthioalkyl, alkylthioalkenyl, alkylthioalkynyl, haloalkylthioalkyl, haloalkylthioalkenyl, haloalkylthioalkynyl, NR³R⁴, SO₂NR³R⁴, carboxyalkyl, carboxyalkenyl, carboxyalkynyl, dialkoxyphosphorylalkyl, aryl, aryl substituted with one or more substituents independently selected from halo, nitro, hydroxy, cyano, thiocyanato, alkyl, alkylsulfonylalkyl, alkenyl, alkynyl, haloalkyl, haloalkenyl, haloalkynyl, alkoxy, haloalkoxy, SO₂NR³R⁴ and NR³R⁴, aralkyl, aralkenyl, aralkynyl, arcycloalkyl, aroxyalkyl, or aralkyl, aralkenyl, aralkynyl, arcycloalkyl, aroxyalkyl substituted with one or more substituents independently selected from halo, nitro, hydroxy, cyano, alkyl, cycloalkyl, alkenyl, alkynyl, haloalkyl, haloalkenyl, haloalkynyl, alkoxy, haloalkoxy, SO₂NR³R⁴ and NR³R⁴, heteroaryl, heteroaryl substituted with one or more substituents independently selected from halo, nitro, hydroxy, cyano, alkyl, alkenyl, alkynyl, haloalkyl, haloalkenyl, haloalkynyl, alkoxy, haloalkoxy, SO₂NR³R⁴ and NR³R⁴, heteroaralkyl, heteroaralkenyl, heteroaralkynyl, or heteroaralkyl, heteroaralkenyl, heteroaralkynyl substituted with one or more substituents independently selected from halo, hydroxy, nitro, cyano, alkyl, alkenyl, alkynyl, haloalkyl, haloalkenyl, haloalkynyl, alkoxy, haloalkoxy, SO₂NR³R⁴ and NR³R⁴, alkylcarbonylalkyl, alkenylcarbonylalkyl, alkynylcarbonylalkyl, heterocyclylcarbonyl, heterocyclylcarbonylalkyl, heterocyclyloxycarbonylalkyl, arylcarbonyl, arylcarbonylalkyl, aralkylcarbonyl, aralkylcarbonylalkyl, aroxycarbonylalkyl, aralkoxycarbonylalkyl, heteroarylcarbonyl, heteroarylcarbonylalkyl, heteroaroxycarbonylalkyl, or arylcarbonyl, arylcarbonylalkyl, aralkylcarbonyl, aralkylcarbonylalkyl, aroxycarbonylalkyl, aralkoxycarbonylalkyl, heteroarylcarbonyl, heteroarylcarbonylalkyl, heteroaroxycarbonylalkyl substituted with one or more substituents independently selected from halo, hydroxy, nitro, cyano, alkyl, alkenyl, alkynyl, haloalkyl, haloalkenyl, haloalkynyl, alkoxy, haloalkoxy, SO₂NR³R⁴ and NR³R⁴, and C(═N—G²²)R²,

G²² is OR³, OCOR³, S(O)ⱼR³, OS(O)ⱼR³, NR³R⁴, OSO₂NR³R⁴, OP(═O)OR³NR³R⁴, OP(═O)(OR³)₂ or N═CR³R⁴,

j is 0, 1 or 2,

R² is a hydrogen atom, alkyl, alkenyl, alkynyl, alkoxyalkyl, alkoxyalkenyl, alkoxyalkynyl, alkylthioalkyl, alkylthioalkenyl, alkylthioalkynyl, carboxy, a carboxylate salt, carboxyalkyl, carboxyalkenyl, carboxyalkynyl, alkoxycarbonyl, alkoxycarbonylalkyl, alkoxycarbonylalkenyl, alkoxycarbonylalkynyl, alkylcarbonyl, alkenylcarbonyl, alkynylcarbonyl, cycloalkyl, cycloalkenyl, cycloalkylalkyl, cycloalkenylalkyl, cycloalkylalkenyl, cycloalkenylalkenyl, cycloalkylalkynyl, cycloalkenylalkynyl, heterocyclyl, heterocyclylalkyl, heterocyclylalkenyl, heterocyclylalkynyl, or alkyl, alkenyl, alkynyl, alkoxyalkyl, alkoxyalkenyl, alkoxyalkynyl, alkylthlioalkyl, alkylthioalkenyl, alkylthioalkynyl, carboxyalkyl, carboxyalkenyl, carboxyalkynyl, alkoxycarbonyl, alkoxycarbonylalkyl, alkoxycarbonylalkenyl, alkoxycarbonylalkynyl, alkylcarbonyl, alkenylcarbonyl, alkynylcarbonyl, cycloalkyl, cycloalkenyl, cycloalkylalkyl, cycloalkenylalkyl, cycloalkylalkenyl, cycloalkenylalkenyl, cycloalkylalkynyl, cycloalkenylalkynyl, heterocyclyl, heterocyclylalkyl, heterocyclylalkenyl, heterocyclylalkynyl substituted with one or more substituents independently selected from halo, cyano, hydroxy, nitro, SO₂NR³R⁴ and NR³R⁴, aryl or aryl substituted with one or more substituents independently selected from halo, hydroxy, cyano, nitro, alkyl, alkenyl, alkynyl, haloalkyl, haloalkenyl, haloalkynyl, alkoxy, haloalkoxy, carboxy, alkoxycarbonyl, SO₂NR³R⁴ and NR³R⁴, aralkyl, aralkenyl, aralkynyl or aralkyl, aralkenyl, aralkynyl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, arylcarbonyl, aralkylcarbonyl, aralkenylcarbonyl, aralkynylcarbonyl or arylcarbonyl, aralkylcarbonyl, aralkenylcarbonyl, aralkynylcarbonyl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, heteroaryl or heteroaryl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, heteroaralkyl, heteroaralkenyl, heteroaralkynyl or heteroaralkyl, heteroaralkenyl, heteroaralkynyl substituted with one or more substituents independently selected from halo, cyano, hydroxy, nitro, alkyl, alkenyl, alkynyl, haloalkyl, haloalkenyl, haloalkynyl, alkoxy, haloalkoxy, SO₂NR³R⁴ and NR³R⁴, heteroaralkylcarbonyl, heteroaralkenylcarbonyl, heteroaralkynylcarbonyl or heteroaralkylcarbonyl, heteroaralkenylcarbonyl, heteroaralkynylcarbonyl substituted with one or more substituents independently selected from halo, cyano, hydroxy, nitro, alkyl, alkenyl, alkynyl, haloalkyl, haloalkenyl, haloalkynyl, alkoxy, haloalkoxy, SO₂NR³R⁴ and NR³R⁴,

R¹² is alkyl, cycloalkyl, cycloalkenyl, cycloalkylalkyl, cycloalkylalkenyl, cycloalkylalkynyl, cycloalkenylalkyl, cycloalkenylalkenyl, cycloalkenylalkynyl, carboxyalkyl, carboxyalkenyl, carboxyalkynyl, heterocyclyl, heterocyclylalkyl, heterocyclylalkenyl, heterocyclylalkynyl, alkoxyalkyl, alkenyl, alkynyl, or alkyl, cycloalkyl, cycloalkenyl, cycloalkylalkyl, cycloalkylalkenyl, cycloalkylalkynyl, cycloalkenylalkyl, cycloalkenylalkenyl, cycloalkenylalkynyl, carboxyalkyl, carboxyalkenyl, carboxyalkynyl, heterocyclyl, heterocyclylalkyl, heterocyclylalkenyl, heterocyclylalkynyl, alkoxyalkyl, alkenyl or alkynyl substituted with one or more halo, aryl or aryl substituted with one or more substituents independently selected from halo, nitro, cyano, haloalkyl, haloalkenyl, haloalkynyl, alkylcarbonyl, alkenylcarbonyl, alkynylcarbonyl, arylcarbonyl, alkylsulfonyl and arylsulfonyl, aralkyl, aralkenyl, aralkynyl, or aralkyl, aralkenyl, aralkynyl substituted with one or more substituents independently selected from halo, alkyl, alkenyl, alkynyl, haloalkyl, haloalkenyl, haloalkynyl, alkoxy and haloalkoxy, heteroaryl, heteroaralkyl, heteroaralkenyl, heteroaralkynyl, or heteroaryl, heteroaralkyl, heteroaralkenyl, heteroaralkynyl substituted with one or more substituents independently selected from halo, alkyl, alkenyl, alkynyl, haloalkyl, haloalkenyl, haloalkynyl, alkoxy and haloalkoxy,

R³, R⁴ and R⁵ are each independently a hydrogen atom, alkyl, cycloalkyl, cycloalkenyl, cycloalkylalkyl, cycloalkylalkenyl, cycloalkylalkynyl, cycloalkenylalkyl, cycloalkenylalkenyl, cycloalkenylalkynyl, carboxyalkyl, carboxyalkenyl, carboxyalkynyl, heterocyclyl, heterocyclylalkyl, heterocyclylalkenyl, heterocyclylalkynyl, alkoxyalkyl, alkenyl, alkynyl, or alkyl, cycloalkyl, cycloalkenyl, cycloalkylalkyl, cycloalkylalkenyl, cycloalkylalkynyl, cycloalkenylalkyl, cycloalkenylalkenyl, cycloalkenylalkynyl, carboxyalkyl, carboxyalkenyl, carboxyalkynyl, heterocyclyl, heterocyclylalkyl, heterocyclylalkenyl, heterocyclylalkynyl, alkoxyalkyl, alkenyl or alkynyl substituted with one or more halo, aryl, aralkyl, aralkenyl, aralkynyl, or aryl, aralkyl, aralkenyl, aralkynyl substituted with one or more substituents independently selected from halo, alkyl, alkenyl, alkynyl, haloalkyl, haloalkenyl, haloalkynyl, alkoxy and haloalkoxy, heteroaryl, heteroaralkyl, heteroaralkenyl, heteroaralkynyl, or heteroaryl, heteroaralkyl, heteroaralkenyl, heteroaralkynyl substituted with one or more substituents independently selected from halo, alkyl, alkenyl, alkynyl, haloalkyl, haloalkenyl, haloalkynyl, alkoxy and haloalkoxy, or R³ and R⁴ taken together with the nitrogen atom to which they are attached form a 5-or 6-membered saturated or unsaturated heterocyclic ring and

Y¹ is halo.

In a preferred mode of this third embodiment,

Y¹ is chloro,

R¹² is alkyl, aralkyl, aryl, heteroaryl, or aralkyl, aryl or heteroaryl substituted with one or more substituents independently selected from halo, alkyl, alkenyl, alkynyl, haloalkyl, haloalkenyl, haloalkynyl, alkoxy and haloalkoxy,

Z³ is alkyl, aryl, aralkyl, or aryl or aralkyl substituted with one or more substituents independently selected from halo, alkyl, alkenyl, alkynyl, haloalkyl, haloalkenyl, haloalkynyl, alkoxy and haloalkoxy, and

R² is a hydrogen atom.

In a more preferred mode of this fourth embodiment, R¹² is alkyl and Z³ is alkyl or aralkyl.

In another more preferred mode of this fourth embodiment,

Z³ is (C₁-C₂₀)alkyl, (C₁-C₁₀)alkylcarbonyloxy(C₁-C₁₀)alkyl, hydroxy(C₁-C₂₀) alkyl, (C₁-C₁₀)alkylsulfonyl(C₁-C₁₀)alkyl, acetylamino(C₁-C₁₀)alkyl, halo(C₁-C₂₀) alkyl, (C₂-C₁₀)alkenyl, acetylamino(C₂-C₁₀)alkenyl, halo(C₂-C₁₀)alkenyl, (C₂-C₁₀) alkynyl, halo(C₂-C₁₀)alkynyl, cyclo(C₃-C₈)alkyl, cyclo(C₃-C₈)alkenyl, carboxycyclo(C₃-C₈)alkyl, carboxycyclo(C₃-C₈)alkenyl, cyclo(C₃-C₈)alkyl(C₁-C₁₀) alkyl, cyclo(C₃-C₈)alkyl(C₂-C₁₀)alkenyl, cyclo(C₃-C₈)alkenyl(C₁-C₁₀)alkyl, cyclo(C₃-C₈)alkenyl(C₂-C₁₀)alkenyl, cyclo(C₃-C₈)alkyl(C₂-C₁₀)alkynyl, cyclo(C₃-C₈) alkenyl(C₂-C₁₀)alkynyl, carboxycyclo(C₃-C₈)alkyl(C₁-C₁₀)alkyl, carboxycyclo(C₃-C₈)alkyl(C₂-C₁₀)alkenyl, carboxycyclo(C₃-C₈)alkenyl(C₁-C₁₀)alkyl, carboxycyclo(C₃-C₈)alkenyl(C₂-C₁₀)alkenyl, carboxycyclo(C₃-C₈)alkyl(C₂-C₁₀) alkynyl, carboxycyclo(C₃-C₈)alkenyl(C₂-C₁₀)alkynyl, heterocyclyl, heterocyclyl(C₁-C₁₀)alkyl, heterocyclyl(C₂-C₁₀)alkenyl, heterocyclyl(C₂-C₁₀) alkynyl, (C₁-C₁₀)alkoxy(C₁-C₁₀)alkyl, (C₁-C₁₀)alkoxy(C₁-C₁₀)alkoxy(C₁-C₁₀) alkyl, (C₁-C₁₀)alkoxy(C₂-C₁₀)alkenyl, (C₁-C₁₀)alkoxy(C₂-C₁₀)alkynyl, (C₁-C₁₀) alkoxycarbonyl(C₁-C₁₀)alkyl, (C₁-C₁₀)alkoxycarbonyl(C₂-C₁₀)alkenyl, (C₁-C₁₀) alkoxycarbonyl(C₂-C₁₀)alkynyl, halo(C₁-C₁₀)alkoxy(C₁-C₁₀)alkyl, halo(C₁-C₁₀) alkoxy(C₂-C₁₀)alkenyl, halo(C₁-C₁₀)alkoxy(C₂-C₁₀)alkynyl, (C₁-C₁₀) alkylthio(C₁-C₁₀)alkyl, (C₁-C₁₀)alkylthio(C₂-C₁₀)alkenyl, (C₁-C₁₀)alkylthio(C₂-C₁₀) alkynyl, halo(C₁-C₁₀)alkylthio(C₁-C₁₀)alkyl, halo(C₁-C₁₀)alkylthio(C₂-C₁₀) alkenyl, halo(C₁-C₁₀)alkylthio(C₂-C₁₀)alkynyl, carboxy(C₁-C₂₀)alkyl, carboxy(C₂-C₁₀)alkenyl, carboxy(C₂-C₁₀)alkynyl, SiR³R⁴R⁵, aryl, aryl substituted with one or more substituents independently selected from halo, nitro, hydroxy, cyano, thiocyanato, (C₁-C₁₀)alkyl, (C₁-C₁₀)alkylsulfonyl(C₁-C₁₀)alkyl, (C₂-C₁₀) alkenyl, (C₂-C₁₀)alkynyl, halo(C₁-C₁₀)alkyl, halo(C₂-C₁₀)alkenyl, halo(C₂-C₁₀) alkynyl, (C₁-C₁₀)alkoxy, halo(C₁-C₁₀)alkoxy, SO₂NR³R⁴ and NR³R⁴, ar(C₁-C₁₀) alkyl, ar(C₂-C₁₀)alkenyl, ar(C₂-C₁₀)alkynyl, arcyclo(C₃-C₈)alkyl, aroxy(C₁-C₁₀) alkyl, or ar(C₁-C₁₀)alkyl, ar(C₂-C₁₀)alkenyl, ar(C₂-C₁₀)alkynyl, arcyclo(C₃-C₈) alkyl, aroxy(C₁-C₁₀)alkyl substituted with one or more substituents independently selected from halo, nitro, hydroxy, cyano, (C₁-C₁₀)alkyl, cycloalkyl, (C₂-C₁₀)alkenyl, (C₂-C₁₀)alkynyl, halo(C₁-C₁₀)alkyl, halo(C₂-C₁₀)alkenyl, halo(C₂-C₁₀) alkynyl, (C₁-C₁₀)alkoxy, halo (C₁-C₁₀)alkoxy, SO₂NR³R⁴ and NR³R⁴, heteroaryl, heteroaryl substituted with one or more substituents independently selected from halo, nitro, hydroxy, cyano, (C₁-C₁₀)alkyl, (C₂-C₁₀)alkenyl, (C₂-C₁₀) alkynyl, halo(C₁-C₁₀)alkyl, halo(C₂-C₁₀)alkenyl, halo(C₂-C₁₀)alkynyl, (C₁-C₁₀) alkoxy, halo(C₁-C₁₀)alkoxy, SO₂NR³R⁴ and NR³R⁴, heteroar(C₁-C₁₀)alkyl, heteroar(C₂-C₁₀)alkenyl, heteroar(C₂-C₁₀)alkynyl, or heteroar(C₁-C₁₀)alkyl, heteroar(C₂-C₁₀)alkenyl, heteroar(C₂-C₁₀)alkynyl substituted with one or more substituents independently selected from halo, hydroxy, cyano, nitro, (C₁-C₁₀) alkyl, (C₂-C₁₀)alkenyl, (C₂-C₁₀)alkynyl, halo(C₁-C₁₀)alkyl, halo (C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴,

Z² is a hydrogen atom, (C₁-C₂₀)alkyl, (C₁-C₁₀)alkylcarbonyloxy(C₁-C₁₀) alkyl, (C₁-C₂₀)alkylcarbonyl, (C₁-C₂₀)alkenylcarbonyl, (C₁-C₂₀) alkynylcarbonyl, hydroxy(C₁-C₂₀)alkyl, (C₁-C₁₀)alkylsulfonyl(C₁-C₁₀)alkyl, acetylamino(C₁-C₁₀)alkyl, halo(C₁-C₂₀)alkyl, (C₂-C₂₀)alkenyl, halo(C₂-C₂₀)alkenyl, acetylamino(C₂-C₁₀)alkenyl, (C₂-C₂₀)alkynyl, halo(C₂-C₂₀)alkynyl, cyclo(C₃-C₈) alkyl, cyclo(C₃-C₈)alkenyl, carboxycyclo(C₃-C₈)alkyl, carboxycyclo(C₃-C₈) alkenyl, cyclo(C₃-C₈)alkyl(C₁-C₁₀)alkyl, cyclo(C₃-C₈)alkyl(C₂-C₁₀)alkenyl, cyclo(C₃-C₈)alkenyl(C₁-C₁₀)alkyl, cyclo(C₃-C₈)alkenyl(C₂-C₁₀)alkenyl, cyclo(C₃-C₈) alkyl(C₂-C₁₀)alkynyl, cyclo(C₃-C₈)alkenyl(C₂-C₁₀)alkynyl, carboxycyclo(C₃-C₈) alkyl(C₁-C₁₀)alkyl, carboxy(C₃-C₈)cycloalkyl(C₂-C₁₀)alkenyl, carboxycyclo(C₃-C₈) alkenyl(C₁-C₁₀)alkyl, carboxycyclo(C₃-C₈)alkenyl(C₂-C₁₀)alkenyl, carboxycyclo(C₃-C₈)alkyl(C₂-C₁₀)alkynyl, carboxycyclo(C₃-C₈)alkenyl(C₂-C₁₀) alkynyl, heterocyclyl, heterocyclyl(C₁-C₁₀)alkyl, heterocyclyl(C₂-C₁₀)alkenyl, heterocyclyl(C₂-C₁₀)alkynyl, (C₁-C₁₀)alkoxy(C₁-C₁₀)alkyl, (C₁-C₈)alkoxy(C₁-C₅) alkoxy(C₁-C₁₀)alkyl, (C₁-C₁₀)alkoxy(C₂-C₁₀)alkenyl, (C₁-C₁₀)alkoxy(C₂-C₁₀) alkynyl, (C₁-C₁₀)alkoxycarbonyl(C₁-C₁₀)alkyl, (C₁-C₁₀)alkoxycarbonyl(C₂-C₁₀) alkenyl, (C₁-C₁₀)alkoxycarbonyl(C₂-C₁₀)alkynyl, halo(C₁-C₁₀)alkoxy(C₁-C₁₀) alkyl, halo(C₁-C₁₀)alkoxy(C₂-C₁₀)alkenyl, halo(C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀) alkylthio(C₂-C₁₀)alkynyl, halo(C₁-C₁₀)alkylthio(C₁-C₁₀)alkyl, halo(C₁-C₁₀) alkylthio(C₂-C₁₀)alkenyl, halo(C₁-C₁₀)alkylthio(C₂-C₁₀)alkynyl, SO₂NR³R⁴, NR³R⁴, carboxy(C₁-C₂₀)alkyl, carboxy(C₂-C₂₀)alkenyl, carboxy(C₂-C₂₀)alkynyl, di(C₁-C₁₀)alkoxyphosphoryl(C₁-C₁₀)alkyl, aryl, aryl substituted with one or more substituents independently selected from halo, nitro, cyano, hydroxy, (C₁-C₁₀) alkyl, (C₁-C₁₀)alkylsulfonyl(C₁-C₁₀)alkyl, (C₁-C₁₀)alkylsulfonyl, thiocyanato, (C₂-C₁₀)alkenyl, (C₂-C₁₀)alkynyl, halo(C₁-C₁₀)alkyl, halo(C₂-C₁₀)alkenyl, halo(C₂-C₁₀) alkynyl, (C₁-C₁₀)alkoxy, halo(C₁-C₁₀)alkoxy, SO₂NR³R⁴, and NR³R⁴, ar(C₁-C₁₀) alkyl, ar(C₂-C₁₀)alkenyl, ar(C₂-C₁₀)alkynyl, arcyclo(C₃-C₈)alkyl, aroxy(C₁-C₁₀) alkyl, or ar(C₁-C₁₀)alkyl, ar(C₂-C₁₀)alkenyl, ar(C₂-C₁₀)alkynyl, arcyclo(C₃-C₈) alkyl, aroxy(C₁-C₁₀)alkyl substituted with one or more substituents independently selected from halo, nitro, hydroxy, cyano, (C₁-C₁₀)alkyl, cyclo(C₃-C₈) alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroaryl, heteroaryl substituted with one or more substituents independently selected from halo, hydroxy, nitro, cyano, (C₁-C₁₀)alkyl, (C₂-C₁₀)alkenyl, (C₂-C₁₀) alkynyl, halo(C₁-C₁₀)alkyl, halo(C₂-C₁₀)alkenyl, halo(C₂-C₁₀)alkynyl, (C₁-C₁₀) alkoxy, halo(C₁-C₁₀)alkoxy and NR³R⁴, heteroar(C₁-C₁₀)alkyl, heteroar(C₂-C₁₀) alkenyl, heteroar(C₂-C₁₀)alkynyl, or heteroar(C₁-C₁₀)alkyl, heteroar(C₂-C₁₀) alkenyl, heteroar(C₂-C₁₀)alkynyl substituted with one or more substituents independently selected from halo, hydroxy, cyano, nitro, (C₁-C₁₀)alkyl, (C₂-C₁₀) alkenyl, (C₂-C₁₀)alkynyl, halo(C₁-C₁₀)alkyl, halo(C₂-C₁₀)alkenyl, halo(C₂-C₁₀) alkynyl, (C₁-C₁₀)alkoxy, halo(C₁-C₁₀)alkoxy, SO₂NR³R⁴ and NR³R⁴, (C₁-C₁₀) alkylcarbonyl(C₁-C₁₀)alkyl, (C₂-C₁₀)alkenylcarbonyl(C₁-C₁₀)alkyl, (C₂-C₁₀) alkynylcarbonyl(C₁-C₁₀)alkyl, heterocyclylcarbonyl, heterocyclylcarbonyl(C₁-C₁₀) alkyl, heterocyclyloxycarbonyl(C₁-C₁₀)alkyl, arylcarbonyl, arylcarbonyl(C₁-C₁₀) alkyl, ar(C₁-C₁₀)alkylcarbonyl, ar(C₁-C₁₀)alkylcarbonyl(C₁-C₁₀)alkyl, aroxycarbonyl(C₁-C₁₀)alkyl, ar(C₁-C₁₀)alkoxycarbonyl(C₁-C₁₀)alkyl, heteroarylcarbonyl, heteroarylcarbonyl(C₁-C₁₀)alkyl, heteroaroxycarbonyl(C₁-C₁₀) alkyl, or arylcarbonyl, arylcarbonyl(C₁-C₁₀)alkyl, ar(C₁-C₁₀)alkylcarbonyl, ar(C₁-C₁₀)alkylcarbonyl(C₁-C₁₀)alkyl, aroxycarbonyl(C₁-C₁₀)alkyl, ar(C₁-C₁₀)alkoxycarbonyl(C₁-C₁₀)alkyl, heteroarylcarbonyl, heteroarylcarbonyl(C₁-C₁₀) alkyl, heteroaroxycarbonyl(C₁-C₁₀)alkyl substituted with one or more substituents independently selected from halo, hydroxy, nitro, cyano, (C₁-C₁₀) alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, and C(═N—G²²)R²,

G²² is OR³, OCOR³, S(O)ⱼR³, OS(O)ⱼR³, NR³R⁴, OSO₂NR³R⁴, OP(═O)OR³NR³R⁴, OP(═O)(OR³)₂ or N═CR³R⁴,

j is 0, 1 or 2,

R² is a hydrogen atom, (C₁-C₂₀)alkyl, (C₂-C₁₀)alkenyl, (C₂-C₁₀)alkynyl, (C₁-C₁₀) alkoxy(C₁-C₁₀)alkyl, (C₁-C₁₀)alkoxy(C₂-C₁₀)alkenyl, (C₁-C₁₀)alkoxy(C₂-C₁₀) alkynyl, (C₁-C₁₀)alkylthio(C₁-C₁₀)alkyl, (C₁-C₁₀)alkylthio(C₂-C₁₀)alkenyl, (C₁-C₁₀) alkylthio(C₂-C₁₀)alkynyl, carboxy, a carboxylate salt, carboxy(C₁-C₂₀)alkyl, carboxy(C₂-C₂₀)alkenyl, carboxy(C₂-C₂₀)alkynyl, (C₁-C₂₀)alkoxycarbonyl, (C₁-C₁₀) alkoxycarbonyl(C₁-C₁₀)alkyl, (C₁-C₁₀)alkoxycarbonyl(C₂-C₁₀)alkenyl, (C₁-C₁₀) alkoxycarbonyl(C₂-C₁₀)alkynyl, (C₁-C₂₀)alkylcarbonyl, (C₂-C₂₀) alkenylcarbonyl, (C₂-C₂₀)alkynylcarbonyl, cyclo(C₃-C₈)alkyl, cyclo(C₃-C₈) alkenyl, cyclo(C₃-C₈)alkyl(C₁-C₁₀)alkyl, cyclo(C₃-C₈)alkenyl(C₁-C₁₀)alkyl, cyclo(C₃-C₈)alkyl(C₂-C₁₀)alkenyl, cyclo(C₃-C₈)alkenyl(C₂-C₁₀)alkenyl, cyclo(C₃-C₈) alkyl(C₂-C₁₀)alkynyl, cyclo(C₃-C₈)alkenyl(C₂-C₁₀)alkynyl, heterocyclyl, heterocyclyl(C₁-C₁₀)alkyl, heterocyclyl(C₂-C₁₀)alkenyl, heterocyclyl(C₂-C₁₀) alkynyl, or (C₁-C₂₀)alkyl, (C₂-C₁₀)alkenyl, (C₂-C₁₀)alkynyl, (C₁-C₁₀)alkoxy(C₁-C₁₀) alkyl, (C₁-C₁₀)alkoxy(C₂-C₁₀)alkenyl, (C₁-C₁₀)alkoxy(C₂-C₁₀)alkynyl, (C₁-C₁₀) alkylthio(C₁-C₁₀)alkyl, (C₁-C₁₀)alkylthio(C₂-C₁₀)alkenyl, (C₁-C₁₀)alkylthio(C₂-C₁₀) alkynyl, carboxy(C₁-C₂₀)alkyl, carboxy(C₂-C₁₀)alkenyl, carboxy(C₂-C₁₀)alkynyl, (C₁-C₂₀)alkoxycarbonyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl (C₂-C₁₀)alkynyl, (C₁-C₂₀)alkylcarbonyl, (C₂-C₁₀)alkenylcarbonyl, (C₂-C₁₀)alkynylcarbonyl, cyclo(C₃-C₈) alkyl, cyclo(C₃-C₈)alkenyl, cyclo(C₃-C₈)alkyl(C₁-C₁₀)alkyl, cyclo(C₃-C₈) alkenyl(C₁-C₁₀)alkyl, cyclo(C₃-C₈)alkyl(C₂-C₁₀)alkenyl, cyclo(C₃-C₈)alkenyl(C₂-C₁₀) alkenyl, cyclo(C₃-C₈)alkyl(C₂-C₁₀)alkynyl, cyclo(C₃-C₈)alkenyl(C₂-C₁₀)alkynyl, heterocyclyl, heterocyclyl(C₁-C₁₀)alkyl, heterocyclyl(C₂-C₁₀)alkenyl, heterocyclyl(C₂-C₁₀)alkynyl substituted with one or more substituents independently selected from halo, cyano, hydroxy, nitro, SO₂NR³R⁴ and NR³R⁴, aryl or aryl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, carboxy, (C₁-C₄)alkoxycarbonyl, SO₂NR³R⁴ and NR³R⁴, ar(C₁-C₁₀)alkyl, ar(C₂-C₁₀) alkenyl, ar(C₂-C₁₀)alkynyl, or ar(C₁-C₁₀)alkyl, ar(C₂-C₁₀)alkenyl, ar(C₂-C₁₀) alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, arylcarbonyl, ar(C₁-C₁₀)alkylcarbonyl, ar(C₂-C₁₀) alkenylcarbonyl, ar(C₂-C₁₀)alkynylcarbonyl or arylcarbonyl, ar(C₁-C₁₀) alkylcarbonyl, ar(C₂-C₁₀)alkenylcarbonyl, ar(C₂-C₁₀)alkynylcarbonyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀) alkyl, halo(C₂-C₁₀)alkenyl, halo(C₂-C₁₀)alkynyl, (C₁-C₁₀)alkoxy, halo(C₁-C₁₀) alkoxy, SO₂NR³R⁴ and NR³R⁴, heteroaryl, heteroaryl substituted with one or more substituents independently selected from halo, (C₁-C₁₀)alkyl, (C₂-C₁₀) alkenyl, (C₂-C₁₀)alkynyl, halo(C₁-C₁₀)alkyl, halo(C₂-C₁₀)alkenyl, halo(C₂-C₁₀) alkynyl, (C₁-C₁₀)alkoxy, halo(C₁-C₁₀)alkoxy and NR³R⁴, heteroar(C₁-C₁₀)alkyl, heteroar(C₂-C₁₀)alkenyl, heteroar(C₂-C₁₀)alkynyl or heteroar(C₁-C₁₀)alkyl, heteroar(C₂-C₁₀)alkenyl, heteroar(C₂-C₁₀)alkynyl substituted with one or more substituents independently selected from halo, (C₁-C₁₀)alkyl, (C₂-C₁₀)alkenyl, (C₂-C₁₀) alkynyl, halo(C₁-C₁₀)alkyl, halo(C₂-C₁₀)alkenyl, halo(C₂-C₁₀)alkynyl, (C₁-C₁₀) alkoxy, halo(C₁-C₁₀)alkoxy, SO₂NR³R⁴ and NR³R⁴, heteroar(C₁-C₁₀) alkylcarbonyl, heteroar(C₂-C₁₀)alkenylcarbonyl, heteroar(C₂-C₁₀) alkynylcarbonyl or heteroar(C₁-C₁₀)alkylcarbonyl, heteroar(C₂-C₁₀) alkenylcarbonyl, heteroar(C₂-C₁₀)alkynylcarbonyl substituted with one or more substituents independently selected from halo, cyano, hydroxy, nitro, (C₁-C₁₀) alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, or R¹ and R² taken together with the carbon atom to which they are attached form a 5-7 membered saturated or unsaturated ring,

R³, R⁴ and R⁵ are each independently a hydrogen atom, (C₁-C₂₀)alkyl, cyclo(C₃-C₈)alkyl, cyclo(C₃-C₈)alkenyl, cyclo(C₃-C₈)alkyl(C₁-C₁₀)alkyl, cyclo(C₃-C₈) alkyl(C₂-C₁₀)alkenyl, cyclo(C₃-C₈)alkyl(C₂-C₁₀)alkynyl, cyclo(C₃-C₈)alkenyl(C₁-C₁₀) alkyl, cyclo(C₃-C₈)alkenyl(C₂-C₁₀)alkenyl, cyclo(C₃-C₈)alkenyl(C₂-C₁₀)alkynyl, carboxy(C₁-C₂₀)alkyl, carboxy(C₂-C₁₀)alkenyl, carboxy(C₂-C₁₀)alkynyl, heterocyclyl, heterocyclyl(C₁-C₁₀)alkyl, heterocyclyl(C₂-C₁₀)alkenyl, heterocyclyl(C₂-C₁₀)alkynyl, (C₁-C₁₀)alkoxy(C₁-C₁₀)alkyl, (C₂-C₁₀)alkenyl, (C₂-C₁₀) alkynyl, or (C₁-C₁₀)alkyl, cyclo(C₃-C₈)alkyl, cyclo(C₃-C₈)alkenyl, cyclo(C₃-C₈) alkyl(C₁-C₁₀)alkyl, cyclo(C₃-C₈)alkyl(C₂-C₁₀)alkenyl, cyclo(C₃-C₈)alkyl(C₂-C₁₀) alkynyl, cyclo(C₃-C₈)alkenyl(C₁-C₁₀)alkyl, cyclo(C₃-C₈)alkenyl(C₂-C₁₀)alkenyl, cyclo(C₃-C₈)alkenyl(C₂-C₁₀)alkynyl, carboxy(C₁-C₂₀)alkyl, carboxy(C₂-C₁₀)alkenyl, carboxy(C₂-C₁₀)alkynyl, heterocyclyl, heterocyclyl(C₁-C₁₀)alkyl, heterocyclyl(C₂-C₁₀) alkenyl, heterocyclyl(C₂-C₁₀)alkynyl, (C₁-C₁₀)alkoxy(C₁-C₁₀)alkyl, (C₂-C₁₀) alkenyl or (C₂-C₁₀)alkynyl substituted with one or more halo, aryl, ar(C₁-C₁₀) alkyl, ar(C₂-C₁₀)alkenyl, ar(C₂-C₁₀)alkynyl or aryl, ar(C₁-C₁₀)alkyl, ar(C₂-C₁₀) alkenyl, ar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy and halo(C₁-C₁₀)alkoxy, heteroaryl, heteroar(C₁-C₁₀)alkyl, heteroar(C₂-C₁₀)alkenyl, heteroar(C₂-C₁₀)alkynyl or heteroaryl, heteroar(C₁-C₁₀)alkyl, heteroar(C₂-C₁₀) alkenyl, heteroar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy and halo(C₁-C₁₀)alkoxy, or R³ and R⁴ taken together with the nitrogen atom to which they are attached form a 5-or 6-membered saturated or unsaturated heterocyclic ring,

R¹² is (C₁-C₂₀)alkyl, cyclo(C₃-C₈)alkyl, cyclo(C₃-C₈)alkenyl, cyclo(C₃-C₈) alkyl(C₁-C₁₀)alkyl, cyclo(C₃-C₈)alkyl(C₂-C₁₀)alkenyl, cyclo(C₃-C₈)alkyl(C₂-C₁₀) alkynyl, cyclo(C₃-C₈)alkenyl(C₁-C₁₀)alkyl, cyclo(C₃-C₈)alkenyl(C₂-C₁₀)alkenyl, cyclo(C₃-C₈)alkenyl(C₂-C₁₀)alkynyl, carboxy(C₁-C₂₀)alkyl, carboxy(C₂-C₂₀)alkenyl, carboxy(C₂-C₂₀)alkynyl, heterocyclyl, heterocyclyl(C₁-C₁₀)alkyl, heterocyclyl(C₂-C₁₀) alkenyl, heterocyclyl(C₂-C₁₀)alkynyl, (C₁-C₁₀)alkoxy(C₁-C₁₀)alkyl, (C₂-C₂₀) alkenyl, (C₂-C₂₀)alkynyl, or (C₁-C₂₀)alkyl, cyclo(C₃-C₈)alkyl, cyclo(C₃-C₈) alkenyl, cyclo(C₃-C₈)alkyl(C₁-C₁₀)alkyl, cyclo(C₃-C₈)alkyl(C₂-C₁₀)alkenyl, cyclo(C₃-C₈)alkyl(C₂-C₁₀)alkynyl, cyclo(C₃-C₈)alkenyl(C₁-C₁₀)alkyl, cyclo(C₃-C₈) alkenyl(C₂-C₁₀)alkenyl, cyclo(C₃-C₈)alkenyl(C₂-C₁₀)alkynyl, carboxy(C₁-C₂₀) alkyl, carboxy(C₂-C₂₀)alkenyl, carboxy(C₂-C₂₀)alkynyl, heterocyclyl, heterocyclyl(C₁-C₁₀)alkyl, heterocyclyl(C₂-C₁₀)alkenyl, heterocyclyl(C₂-C₁₀) alkynyl, (C₁-C₁₀)alkoxy(C₁-C₁₀)alkyl, (C₂-C₂₀)alkenyl or (C₂-C₂₀)alkynyl substituted with one or more halo, aryl or aryl substituted with one or more substituents independently selected from halo, nitro, cyano, halo(C₁-C₁₀)alkyl, halo(C₂-C₁₀)alkenyl, halo(C₂-C₁₀)alkynyl, (C₁-C₁₀)alkylcarbonyl, (C₂-C₁₀) alkenylcarbonyl, (C₂-C₁₀)alkynylcarbonyl, arylcarbonyl, (C₁-C₁₀)alkylsulfonyl and arylsulfonyl, ar(C₁-C₁₀)alkyl, ar(C₂-C₁₀)alkenyl, ar(C₂-C₁₀)alkynyl, or ar(C₁-C₁₀) alkyl, ar(C₂-C₁₀)alkenyl, ar(C₂-C₁₀)alkynyl substituted with one or more substituents independently selected from halo, (C₁-C₁₀)alkyl, (C₂-C₁₀)alkenyl, (C₂-C₁₀) alkynyl, halo(C₁-C₁₀)alkyl, halo(C₂-C₁₀)alkenyl, halo(C₂-C₁₀)alkynyl, (C₁-C₁₀) alkoxy and halo(C₁-C₁₀)alkoxy, heteroaryl, heteroar(C₁-C₁₀)alkyl, heteroar(C₂-C₁₀)alkenyl, heteroar(C₂-C₁₀)alkynyl, or heteroaryl, heteroar(C₁-C₁₀) alkyl, heteroar(C₂-C₁₀)alkenyl, heteroar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀) alkynyl, (C₁-C₁₀)alkoxy and halo(C₁-C₁₀)alkoxy, and

Y¹ is chloro.

In an even more preferred mode of this fourth embodiment,

R¹² is (C₁-C₂₀)alkyl, ar(C₁-C₁₀)alkyl, aryl, heteroaryl, or ar(C₁-C₁₀)alkyl, aryl or heteroaryl substituted with one or more substituents independently selected from halo, (C₁-C₁₀)alkyl, (C₂-C₁₀)alkenyl, (C₂-C₁₀)alkynyl, halo(C₁-C₁₀) alkyl, halo(C₂-C₁₀)alkenyl, halo(C₂-C₁₀)alkynyl, (C₁-C₁₀)alkoxy and halo(C₁-C₁₀)alkoxy,

Z³ is (C₁-C₂₀)alkyl, aryl, ar(C₁-C₁₀)alkyl, or aryl or ar(C₁-C₁₀)alkyl substituted with one or more substituents independently selected from halo, (C₁-C₁₀) alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy and halo (C₁-C₁₀)alkoxy, and

R² is a hydrogen atom.

In yet an even more preferred mode of this fourth embodiment, R¹² is (C₁-C₁₀) alkyl and Z³ is (C₁-C₁₀)alkyl or ar(C₁-C₆)alkyl.

In all of the modes of this fourth embodiment, suitable halogenating agents include, but are not limited to, chlorine gas, thionyl chloride and sulfuryl chloride. A preferred halogenating agent is sulfuryl chloride. Suitable solvents for use in the above process include, but are not limited to, aliphatic hydrocarbons such as hexanes, chlorinated solvents such as methylene chloride, dichloroethane, chloroform, carbon tetrachloride and the like. However, the reactions are normally run neat. The process may be carried out at temperatures from about −78° C. to about 100° C., preferably, from about 0° C. to about 50° C. The process is preferably carried out at about atmospheric pressure although higher or lower pressures can be used if desired. Substantially equimolar amounts of reactants are preferably used although higher or lower amounts can be used if desired. From about 0.9 to about 1.1 equivalents of the halogenating agent are used per equivalent of a compound of formula (II).

A fifth embodiment of this invention is the preparation of a compound of formula (I) comprising the steps of

(i) reacting a compound of formula (VII) with a compound of formula (VIII) to form a compound of formula (V),

(ii) reacting a compound of formula (V) with a compound of formula (VI) to form a compound of formula (III),

(iii) reacting a compound of formula (III) with a compound of formula (IV) to form a compound of formula (II), and

(iv) reacting a compound of formula (II) with a halogenating agent to form a compound of formula (I) as described in each of the first four embodiments, respectively.

In all embodiments of this invention, the term “alkyl” includes both branched and straight chain alkyl groups. Typical alkyl groups are methyl, ethyl, n-propyl, isopropyl, n-butyl, sec-butyl, isobutyl, tert-butyl, n-pentyl, isopentyl, n-hexyl, n-heptyl, isooctyl, nonyl, decyl, undecyl, dodecyl, tetradecyl, hexadecyl, octadecyl, eicosyl and the like.

The term “halo” refers to fluoro, chloro, bromo or iodo.

The term “haloalkyl” refers to an alkyl group substituted with one or more halo groups, for example chloromethyl, 2-bromoethyl, 3-iodopropyl, trifluoromethyl, perfluoropropyl, 8-chlorononyl and the like.

The term “cycloalkyl” refers to a cyclic aliphatic ring structure, optionally substituted with alkyl, hydroxy and halo, such as cyclopropyl, methylcyclopropyl, cyclobutyl, 2-hydroxycyclopentyl, cyclohexyl, 4-chlorocyclohexyl, cycloheptyl, cyclooctyl and the like.

The term “alkylcarbonyloxyalkyl” refers to an ester moiety, for example acetoxymethyl, n-butyryloxyethyl and the like.

The term “alkynylcarbonyl” refers to an alkynylketo functionality, for example propynoyl and the like.

The term “hydroxyalkyl” refers to an alkyl group substituted with one or more hydroxy groups, for example hydroxymethyl, 2,3-dihydroxybutyl and the like.

The term “alkylsulfonylalkyl” refers to an alkyl group substituted with an alkylsulfonyl moiety, for example mesylmethyl, isopropylsulfonylethyl and the like.

The term “alkylsulfonyl” refers to a sulfonyl moiety substituted with an alkyl group, for example mesyl, n-propylsulfonyl and the like.

The term “acetylaminoalkyl” refers to an alkyl group substituted with an amide moiety, for example acetylaminomethyl and the like.

The term “acetylaminoalkenyl” refers to an alkenyl group substituted with an amide moiety, for example 2-(acetylamino)vinyl and the like.

The term “alkenyl” refers to an ethylenically unsaturated hydrocarbon group, straight or branched chain, having 1 or 2 ethylenic bonds, for example vinyl, allyl, 1-butenyl, 2-butenyl, isopropenyl, 2-pentenyl and the like.

The term “haloalkenyl” refers to an alkenyl group substituted with one or more halo groups.

The term “cycloalkenyl” refers to a cyclic aliphatic ring structure, optionally substituted with alkyl, hydroxy and halo, having 1 or 2 ethylenic bonds such as methylcyclopropenyl, trifluoromethylcyclopropenyl, cyclopentenyl, cyclohexenyl, 1,4-cyclohexadienyl and the like.

The term “alkynyl” refers to an unsaturated hydrocarbon group, straight or branched, having 1 or 2 acetylenic bonds, for example ethynyl, propargyl and the like.

The term “haloalkynyl” refers to an alkynyl group substituted with one or more halo groups.

The term “alkylcarbonyl” refers to an alkylketo functionality, for example acetyl, n-butyryl and the like.

The term “alkylcarbonylalkyl” refers to an alkylketoalkyl functionality, for example acetonyl and the like.

The term “alkenylcarbonyl” refers to an alkenylketo functionality, for example, propenoyl and the like.

The term “alkenylcarbonylalkyl” refers to an alkenylketoalkyl functionality, for example, propenoylmethyl and the like.

The term “alkynylcarbonyl” refers to an alkynylketoalkyl functionality, for example, propynoyl and the like.

The term “alkynylcarbonylalkyl” refers to an alkynylketoalkyl functionality, for example, propynoylmethyl and the like.

The term “aryl” refers to phenyl or naphthyl which may be optionally substituted. Typical aryl substituents include, but are not limited to, phenyl, 4-chlorophenyl, 4-fluorophenyl, 4-bromophenyl, 3-nitrophenyl, 2-methoxyphenyl, 2-methylphenyl, 3-methyphenyl, 4-methylphenyl, 4-ethylphenyl, 2-methyl-3-methoxyphenyl, 2,4-dibromophenyl, 3,5-difluorophenyl, 3,5-dimethylphenyl, 2,4,6-trichlorophenyl, 4-methoxyphenyl, naphthyl, 2-chloronaphthyl, 2,4-dimethoxyphenyl, 4-(trifluoromethyl)phenyl, 2-carboxyphenyl, 2-methoxycarbonylphenyl, 4-nitrophenyl, 2,4-dinitrophenyl, 2,3,4,5,6-pentafluorophenyl and 2-iodo-4-methylphenyl.

The term “arylcarbonyl” refers to an aryl group as defined previously attached to a keto group, for example benzoyl and the like.

The term “arylcarbonylalkyl” refers to an arylcarbonyl group as defined previously attached to an alkyl group, for example phenacyl and the like.

The term “heteroaryl” refers to a substituted or unsubstituted 5 or 6 membered unsaturated ring containing one, two or three heteroatoms, preferably one or two heteroatoms independently selected from oxygen, nitrogen and sulfur or to a bicyclic unsaturated ring system containing up to 10 atoms including one heteroatom selected from oxygen, nitrogen and sulfur. Examples of heteroaryls include, but is not limited to, 2-, 3- or 4-pyridinyl, pyrazinyl, 2-, 4-, or 5-pyrimidinyl, pyridazinyl, triazolyl, imidazolyl, 2-or 3-thienyl, 2-or 3-furyl, pyrrolyl, oxazolyl, isoxazolyl, thiazolyl, isothiazolyl, oxadiazolyl, thiadiazolyl, quinolyl and isoquinolyl. The heterocyclic ring may be optionally substituted with up to two substituents.

The term “heteroarylcarbonyl” refers to a heteroaryl group as defined above attached to a keto group, for example 2-pyridylcarbonyl and the like.

The term “heteroarylcarbonylalkyl” refers to a heteroarylcarbonyl group as defined above attached to an group, for example 2-pyridylcarbonylmethyl and the like.

The term “aralkyl” is used to describe a group wherein the alkyl chain can be branched or straight chain with the aryl portion, as defined hereinbefore, forming a terminal portion of the aralkyl moiety. Examples of aralkyl groups include, but are not limited to, optionally substituted benzyl, phenethyl, phenpropyl and phenbutyl such as 4-chlorobenzyl, 2,4-dibromobenzyl, 2-methylbenzyl, 2-(3-fluorophenyl)ethyl, 2-(4-methylphenyl)ethyl, 2-(4-(trifluoromethyl)phenyl)ethyl, 2-(2-methoxyphenyl)ethyl, 2-(3-nitrophenyl)ethyl, 2-(2,4-dichlorophenyl)ethyl, 2-(3,5-dimethoxyphenyl)ethyl, 3-phenylpropyl, 3-(3-chlorophenyl)propyl, 3-(2-methylphenyl)propyl, 3-(4-methoxyphenyl)propyl, 3-(4-(trifluoromethyl)phenyl)propyl, 3-(2,4-dichlorophenyl)propyl, 4-phenylbutyl, 4-(4-chlorophenyl)butyl, 4-(2-methylphenyl)butyl, 4-(2,4-dichlorophenyl)butyl, 4-(2-methoxphenyl)butyl and 10-phenyldecyl.

The term “aralkylcarbonyl” refers to an aralkyl group as defined above attached to a keto group, for example phenylacetyl and the like.

The term “aralkylcarbonylalkyl” refers to an aralkylcarbonyl group as defined above attached to an alkyl group, for example phenylacetylmethyl and the like.

The term “aralkoxy” refers to an aryl group as defined above attached to an alkoxy group, for example benzyloxy and the like.

The term “aralkoxycarbonyl” refers to an aralkoxy group as defined above attached to a keto group, for example benzyloxycarbonyl and the like.

The term “aralkoxycarbonylalkyl” refers to an aralkoxycarbonyl group as defined above attached to an alkyl group, for example benzyloxycarbonylmethyl and the like.

The term “arcycloalkyl” is used to describe a group wherein the aryl group is attached to a cycloalkyl group, for example phenylcyclopentyl and the like.

The term “aralkenyl” is used to describe a group wherein the alkenyl chain can be branched or straight chain with the aryl portion, as defined hereinbefore, forming a terminal portion of the aralkenyl moiety, for example styryl (2-phenylvinyl), phenpropenyl and the like.

The term “aralkynyl” is used to describe a group wherein the alkynyl chain can be branched or straight chain with the aryl portion, as defined hereinbefore, forming a terminal portion of the aralkynyl moiety, for example ³- phenyl-1-propynyl and the like.

The term “aroxy” is used to describe an aryl group attached to a terminal oxygen atom. Typical aroxy groups include phenoxy, 3,4-dichlorophenoxy and the like.

The term “aroxycarbonylalkyl” is used to describe an aroxy group as defined above attached to an alkylcarbonyl group, for example phenoxycarbonylmethyl and the like.

The term “heteroaroxycarbonylalkyl” is used to describe a heteroaroxy group as defined above attached to an alkylcarbonyl group, for example pyridyloxycarbonylmethyl and the like.

The term “aroxyalkyl” is used to describe a group wherein an alkyl group is substituted with an aroxy group, for example pentafluorophenoxymethyl and the like.

The term “heteroaroxy” is used to describe an heteroaryl group attached to a terminal oxygen atom. Typical heteroaroxy groups include 4,6-dimethoxypyrimidin-2-yloxy and the like.

The term “heteroaralkyl” is used to describe a group wherein the alkyl chain can be branched or straight chain with the heteroaryl portion, as defined hereinbefore, forming a terminal portion of the heteroaralkyl moiety, for example 3-furylmethyl, thenyl, furfuryl and the like.

The term “heteroaralkenyl” is used to describe a group wherein the alkenyl chain can be branched or straight chain with the heteroaryl portion, as defined hereinbefore, forming a terminal portion of the heteroaralkenyl moiety, for example 3-(4-pyridyl)-1-propenyl.

The term “heteroaralkynyl” is used to describe a group wherein the alkynyl chain can be branched or straight chain with the heteroaryl portion, as defined hereinbefore, forming a terminal portion of the heteroaralkynyl moiety, for example 4-(2-thienyl)-1-butynyl.

The term “heterocyclyl” refers to a substituted or unsubstituted 5 or 6 membered saturated or partially saturated ring containing one, two or three heteroatoms, preferably three nitrogen atoms, two nitrogen atoms and an oxygen or a sulfur atom, one or two heteroatoms independently selected from oxygen, nitrogen and sulfur or to a bicyclic ring system containing up to 10 atoms including one heteroatom selected from oxygen, nitrogen and sulfur wherein the ring containing the heteroatom is saturated. Substituents can include halo, alkyl, haloalkyl, hydroxy, alkoxy, nitro or oxo (═O) on the heterocyclyl ring. Examples of heterocyclyls include, but are not limited to, tetrahydrofuryl, pyrrolidinyl, piperidinyl, tetrahydropyranyl, morpholinyl, piperazinyl, dioxolanyl, dioxanyl, indolinyl, triazolonyl, oxadiazolonyl, thiadiazolonyl, pyrazolonyl, imidazolonyl, pyridonyl, primidinoyl, pyrazinoyl, triazonyl and 5-methyl-6-chromanyl.

The term “heterocyclylcarbonyl” refers to a heterocyclyl group as defined before attached to a keto group, for example tetrahydrofurylcarbonyl and the like.

The term “heterocyclylcarbonylalkyl” refers to a heterocyclylcarbonyl group as defined before attached to an alkyl group, for example tetrahydrofurylcarbonylmethyl and the like.

The term “heterocyclyloxy” refers to a heterocyclyl group as defined above attached to an oxygen atom, for example piperidinoxy and the like.

The term “heterocyclyloxycarbonyl” refers to a heterocyclyloxy group as defined above attached to a keto group, for example piperidinoxycarbonyl and the like.

The term “heterocyclyloxycarbonylalkyl” refers to a heterocyclyloxycarbonyl group as defined above attached to an alkyl group, for example piperidinoxycarbonylmethyl and the like.

The term “heterocyclylalkyl” is used to describe a group wherein the alkyl chain can be branched or straight chain with the heterocyclyl portion, as defined hereinabove, forming a terminal portion of the heterocyclylalkyl moiety, for example 3-piperidinylmethyl and the like.

The term “heterocyclylalkenyl” is used to describe a group wherein the alkenyl chain can be branched or straight chain with the heterocyclyl portion, as defined hereinbefore, forming a terminal portion of the heterocyclylalkenyl moiety, for example 2-morpholinyl-1-propenyl.

The term “heterocyclylalkynyl” is used to describe a group wherein the alkynyl chain can be branched or straight chain with the heterocyclyl portion, as defined hereinnbefore, forming a terminal portion of the heterocyclylalkynyl moiety, for example 2-pyrrolidinyl-1-butynyl.

The term “carboxyalkyl” includes both branched and straight chain alkyl groups as defined hereinbefore attached to a carboxy (—COOH) group.

The term “carboxyalkenyl” includes both branched and straight chain alkenyl groups as defined hereinbefore attached to a carboxy group.

The term “carboxyalkynyl” includes both branched and straight chain alkynyl groups as defined hereinbefore attached to a carboxy group.

The term “carboxycycloalkyl” refers to a carboxy group attached to a cyclic aliphatic ring structure as defined hereinbefore.

The term “carboxycycloalkenyl” refers to a carboxy group attached to a cyclic aliphatic ring structure having 1 or 2 ethylenic bonds as defined hereinbefore.

The term “cycloalkylalkyl” refers to a cycloalkyl group as defined hereinbefore attached to an alkyl group, for example cyclopropylmethyl, cyclohexylethyl and the like.

The term “cycloalkylalkenyl” refers to a cycloalkyl group as defined hereinbefore attached to an alkenyl group, for example cyclohexylvinyl, cycloheptylallyl and the like.

The term “cycloalkylalkynyl” refers to a cycloalkyl group as defined hereinbefore attached to an alkynyl group, for example cyclopropylpropargyl, 4-cyclopentyl-2-butynyl and the like.

The term “cycloalkenylalkyl” refers to a cycloalkenyl group as defined hereinbefore attached to an alkyl group, for example 2-(cyclopenten-1-yl)ethyl and the like.

The term “cycloalkenylalkenyl” refers to a cycloalkenyl group as defined hereinbefore attached to an alkenyl group, for example 1-(cyclohexen-3-yl)allyl and the like.

The term “cycloalkenylalkynyl” refers to a cycloalkenyl group as defined hereinbefore attached to an alkynyl group, for example 1-(cyclohexen-3-yl) propargyl and the like.

The term “carboxycycloalkylalkyl” refers to a carboxy group attached to the cycloalkyl ring portion of a cycloalkylalkyl group as defined hereinbefore.

The term “carboxycycloalkylalkenyl” refers to a carboxy group attached to the cycloalkyl ring portion of a cycloalkylalkenyl group as defined hereinbefore.

The term “carboxycycloalkylalkynyl” refers to a carboxy group attached to the cycloalkyl ring portion of a cycloalkylalkynyl group as defined hereinbefore.

The term “carboxycycloalkenylalkyl” refers to a carboxy group attached to the cycloalkenyl ring portion of a cycloalkenylalkyl group as defined hereinbefore.

The term “carboxycycloalkenylalkenyl” refers to a carboxy group attached to the cycloalkenyl ring portion of a cycloalkenylalkenyl group as defined hereinbefore.

The term “carboxycycloalkenylalkynyl” refers to a carboxy group attached to the cycloalkenyl ring portion of a cycloalkenylalkynyl group as defined hereinbefore.

The term “alkoxy” includes both branched and straight chain alkyl groups attached to a terminal oxygen atom. Typical alkoxy groups include methoxy, ethoxy, n-propoxy, isopropoxy, tert-butoxy and the like.

The term “haloalkoxy” refers to an alkoxy group substituted with one or more halo groups, for example chloromethoxy, trifluoromethoxy, difluoromethoxy, perfluoroisobutoxy and the like.

The term “alkoxyalkoxyalkyl” refers to an alkyl group substituted with an alkoxy moiety which is in turn substituted with a second alkoxy moiety, for example methoxymethoxymethyl, isopropoxymethoxyethyl and the like.

The term “alkylthio” includes both branched and straight chain alkyl groups attached to a terminal sulfur atom, for example methylthio.

The term “haloalkylthio” refers to an alkylthio group substituted with one or more halo groups, for example trifluoromethylthio.

The term “alkoxyalkyl” refers to an alkyl group substituted with an alkoxy group, for example isopropoxymethyl.

The term “alkoxyalkenyl” refers to an alkenyl group substituted with an alkoxy group, for example 3-methoxyallyl.

The term “alkoxyalkynyl” refers to an alkynyl group substituted with an alkoxy group, for example 3-methoxypropargyl.

The term “alkoxycarbonylalkyl” refers to a straight chain or branched alkyl substituted with an alkoxycarbonyl, for example ethoxycarbonylmethyl, 2-(methoxycarbonyl)propyl and the like.

The term “alkoxycarbonylalkenyl” refers to a straight chain or branched alkenyl as defined hereinbefore substituted with an alkoxycarbonyl, for example 4-(ethoxycarbonyl)-2-butenyl and the like.

The term “alkoxycarbonylalkynyl” refers to a straight chain or branched alkynyl as defined hereinbefore substituted with an alkoxycarbonyl, for example 4-(ethoxycarbonyl)-2-butynyl and the like.

The term “haloalkoxyalkyl” refers to a straight chain or branched alkyl as defined hereinbefore substituted with a haloalkoxy, for example 2-chloroethoxymethyl, trifluoromethoxymethyl and the like.

The term “haloalkoxyalkenyl” refers to a straight chain or branched alkenyl as defined hereinbefore substituted with a haloalkoxy, for example 4-(chloromethoxy)-2-butenyl and the like.

The term “haloalkoxyalkynyl” refers to a straight chain or branched alkynyl as defined hereinbefore substituted with a haloalkoxy, for example 4-(2-fluoroethoxy)-2-butynyl and the like.

The term “alkylthioalkyl” refers to a straight chain or branched alkyl as defined hereinbefore substituted with an alkylthio group, for example methylthiomethyl, 3-(isobutylthio)heptyl and the like.

The term “alkylthioalkenyl” refers to a straight chain or branched alkenyl as defined hereinbefore substituted with an alkylthio group, for example 4-(methylthio)-2-butenyl and the like.

The term “alkylthioalkynyl” refers to a straight chain or branched alkynyl as defined hereinbefore substituted with an alkylthio group, for example 4-(ethylthio)-2-butynyl and the like.

The term “haloalkylthioalkyl” refers to a straight chain or branched alkyl as defined hereinbefore substituted with an haloalkylthio group, for example 2-chloroethylthiomethyl, trifluoromethylthiomethyl and the like.

The term “haloalkylthioalkenyl” refers to a straight chain or branched alkenyl as defined hereinbefore substituted with an haloalkylthio group, for example 4-(chloromethylthio)-2-butenyl and the like.

The term “haloalkylthioalkynyl” refers to a straight chain or branched alkynyl as defined hereinbefore substituted with an haloalkylthio group, for example 4-(2-fluoroethylthio)-2-butynyl and the like.

The term “dialkoxyphosphorylalkyl” refers to two straight chain or branched alkoxy groups as defined hereinbefore attached to a pentavalent phosphorous atom, containing an oxo substituent, which is in turn attached to an alkyl, for example diethoxyphosphorylmethyl.

The compounds of Formula I of this invention and the intermediates used in the synthesis of the compounds of this invention can be prepared according to the following method. Scheme 1 can be used when preparing compounds of Formula I as shown.

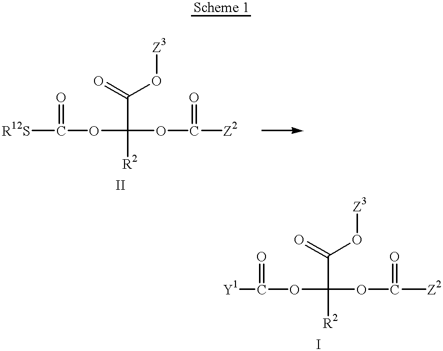

where Z², Z³, R¹² and R² are as defined previously for compound of Formula I and Y¹ is halo.

In a typical preparation, according to Scheme 1, of a compound of Formula I, a compound of Formula II is reacted in a suitable solvent with a suitable halogenating agent. Suitable halogenating agents include chlorine gas, thionyl chloride, and sulfuryl chloride, however, the preferred halogenating agent is sulfuryl chloride. Suitable solvents for use in the above process include, but are not limited to, hexanes, chlorinated solvents such as methylene chloride, dichloroethane, chloroform, carbon tetrachloride and the like, however, the reactions are normally run neat. The above process may be carried out at temperatures between about −78° C. and about 100° C. Preferably, the reaction is carried out between 0° C. and about 50° C. Preparation of the compounds of the present invention by the above process is preferably carried out at about atmospheric pressure although higher or lower pressures can be used if desired. Substantially equimolar amounts of reactants are preferably used although higher or lower amounts can be used if desired.

The compounds of Formula II of Scheme 1 are prepared as shown in Scheme 2.

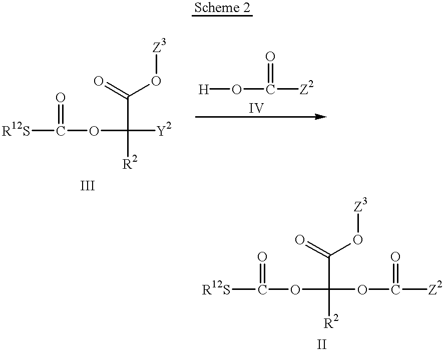

where Z², Z³, R¹² and R² are as defined previously for compound of Formula I and Y² is halogen such as chlorine, bromine, or iodine.

In a typical preparation, according to Scheme 2, of a compound of Formula II, a compound of Formula III is reacted with a compound of Formula IV in a suitable solvent in the presence of a suitable base. Suitable solvents for use in the above process include, but are not limited to, ethers such as tetrahydrofuran (THF), glyme, diethyl ether and the like; dimethylformamide (DMF), dimethylsulfoxide (DMSO), acetonitrile; chlorinated solvents such as methylene chloride (CH₂Cl₂) or chloroform (CHCl₃). If desired, mixtures of these solvents may be used, however, the preferred solvent is THF. Suitable bases for use in the above process include, but are not limited to, metal hydrides such as sodium or potassium hydride; metal alkoxides such as sodium or potassium alkoxides; alkali metal hydroxides such as sodium or potassium hydroxide; tertiary amines such as triethylamine or diisopropylethylamine; an alkali metal carbonate such as sodium or potassium carbonate; or pyridine. If desired, mixtures of these bases may be used, however, the preferred base is diisopropylethylamine. The above process may be carried out at temperatures between about −78° C. and about 100° C. Preferably, the reaction is carried out between 0° C. and about 50° C. Preparation of the compounds of the present invention by the above process is preferably carried out at about atmospheric pressure although higher or lower pressures can be used if desired. Substantially equimolar amounts of reactants are preferably used although higher or lower amounts can be used if desired. Generally, one equivalent of base is used per equivalent of starting material of compound of Formula IV. The compounds of Formula IV are generally commercially available or can be prepared according to known procedures.

The compounds of Formula III of Scheme 2 are prepared as shown in Scheme 3.

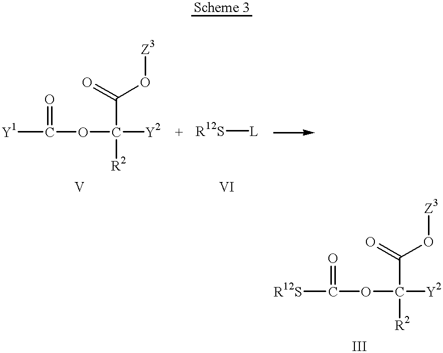

where Z³, R¹² and R² are as defined previously for compound of Formula I, L is a hydrogen atom or a metal cation such as a sodium atom or a potassium atom, and Y¹ and Y² is halogen such as chlorine, bromine, or iodine.

In a typical preparation, according to Scheme 3, of a compound of Formula III, a compound of Formula V is reacted with a compound of Formula VI in a suitable solvent in the presence of a suitable base. Suitable solvents for use in the above process include, but are not limited to, ethers such as tetrahydrofuran (THF), glyme, diethyl ether and the like; dimethylformamide (DMF); dimethylsulfoxide (DMSO); acetonitrile; chlorinated solvents such as methylene chloride (CH₂Cl₂) or chloroform (CHCl₃). If desired, mixtures of these solvents may be used, however, the preferred solvent is diethyl ether. Suitable bases for use in the above process when L is H in compound of formula VI include, but are not limited to, metal hydrides such as sodium or potassium hydride; metal alkoxides such as sodium or potassium alkoxides; alkali metal hydroxides such as sodium or potassium hydroxide; tertiary amines such as triethylamine or diusopropylethylamine; an alkali metal carbonate such as sodium or potassium carbonate; or pyridine. If desired, mixtures of these bases may be used, however, the preferred base is sodium hydride. The above process may be carried out at temperatures between about −78° C. and about 100° C. Preferably, the reaction is carried out between 0° C. and about 50° C. Preparation of the compounds of the present invention by the above process is preferably carried out at about atmospheric pressure although higher or lower pressures can be used if desired. Substantially equimolar amounts of reactants are preferably used although higher or lower amounts can be used if desired. Generally, one equivalent of base is used per equivalent of starting material of compound of Formula VI except when L is a sodium atom or a potassium atom, then no base is required. The compounds of Formula VI are generally commercially available or can be prepared according to known procedures. For example, R¹²S—L is EtS—Na, is commercially available. Conversion of Y² from Cl to Br or Cl to I in compound of Formula III can be prepared according to literature procedures. A general description of the synthesis of halogen exchange (Finkelstein reaction) is described in March, J. Also, see synthesis Example 8 for conversion of Y² from Cl to I in compound of Formula III.

The compounds of Formula V of Scheme 3 are prepared as shown in Scheme 4.

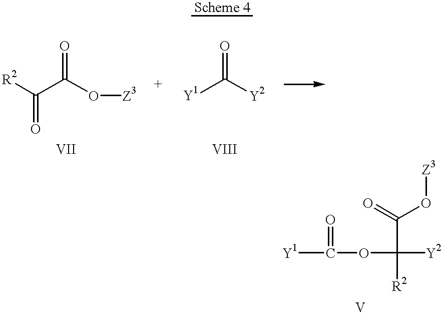

where Z³ and R² are as defined previously for compound of Formula I, and Y¹ and Y² are each independently halo such as chloro or OCCl₃.

In a typical preparation, according to Scheme 4, of a compound of Formula V, a compound of Formula VII is reacted with a compound of Formula VIII in a suitable solvent in the presence of a suitable catalyst. Suitable solvents for use in the above process include, but are not limited to, ethers such as tetrahydrofuran (THF), glyme, diethyl ether, dioxane and the like; acetonitrile; chlorinated solvents such as methylene chloride (CH₂Cl₂), carbon tetrachloride (CCl₄) or chloroform (CHCl₃). If desired, mixtures of these solvents may be used. The preferred solvent is dependent upon the substrates employed and is selected according to the properties of the substrates. For example, in the reaction of compound of Formula VII, where R² is H and Z³ is benzyl (Bn), with compound of Formula VIII, where Y¹ and Y² is Cl, to afford compound of Formula V where R² is H, Z³ is benzyl (Bn), and Y¹ and Y² is Cl, then the preferred solvent is carbon tetrachloride. However, in the reaction of compound of Formula VII, where R² is H and Z³ is ethyl (Et), with compound of Formula VIII, where Y¹ and Y² is Cl, to afford compound of Formula V, where R² is H, Z³ is ethyl (Et), and Y¹ and Y² is Cl, then the preferred solvent is tetrahydrofuran. Suitable catalysts for use in the above process include, but are not limited to, pyridine, thioureas and ureas such as tetra-n-butylurea, phosphoramides such as hexamethylphosphotriamide, substituted amides such as dimethylformamide, quaternary ammonium halides such as tetrabutyl or tributylbenzyl ammonium chloride, arylamines such as N,N-dimethylaminopyridine, N,N-dimethylaniline, tertiary phosphines such as trioctyl phosphine, and alkali metal or alkaline earth metal halides such as cesium or potassium chloride which are used in conjunction with a sequestering agent such as a crown ether (18-crown-6). If desired, mixtures of these catalysts may be used, however, the preferred catalyst is pyridine. Compound of Formula VII may in some cases exist in a polymeric form. If so, the monomeric form can be achieved via known procedures, one being through thermal depolymerization. Compound of Formula VIII in which Y¹ and Y² is Cl is phosgene, C(═O)Cl₂. However, other forms of phosgene, phosgene equivalents, can be utilized such as trichloromethyl chloroformate (compound of Formula VIII in which Y¹ is Cl, and Y² is OCCl₃) or di(trichloromethyl)carbonate (compound of Formula VIII in which Y¹ and Y² are both OCCl₃). The above process may be carried out at temperatures between about −78° C. and about 100° C. Preferably, the reaction is carried out between 0° C. and about 100° C. The above process to produce compounds of the present invention is preferably carried out at about atmospheric pressure although higher or lower pressures can be used if desired. Substantially equimolar amounts of reactants are preferably used although higher or lower amounts can be used if desired. The catalyst is normally used in lower amounts than that of both compounds of Formula VII and VIII. The compounds of Formula VII and VIII are generally commercially available or can be prepared according to known procedures.

Following the general methods described hereinbefore, the following compounds of Formula V as listed in Table 1 were prepared.

TABLE 1

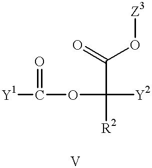

Listing of Compounds of Formula V

| Cmpd# | Y¹ | Y² | R² | Z³ |
|---|---|---|---|---|
| 1-1 | Cl | Cl | H | benzyl |
| 1-2 | Cl | Cl | H | ethyl |
| 1-3 | Cl | Cl | H | methyl |
| 1-4 | Cl | Cl | H | isopropyl |
| 1-5 | Cl | Cl | H | tert-butyl |
| 1-6 | Cl | Cl | H | n-butyl |

The following Examples are provided for guidance to the practitioner in order to practice the invention.

### EXAMPLE 1

Benzyl 2-chloro-2-[(chlorocarbonyl)oxy]Acetate (Compound 1-1 of Table 1)

To a 3-neck round-bottom flask, equipped with nitrogen inlet, a thermometer and a solid addition funnel, was added benzyl glyoxylate (50.9 g, 300 mmol), pyridine (2.5 mL, 31.0 mmol) and 1500 mL of carbon tetrachloride. The solution was cooled with dry ice/acetone to −20° C. and triphosgene (230 g, 770 mmol) was added over 5 minutes, maintaining the temperature between −10° C. and −20° C. The reaction was gradually warmed to room temperature over 2 h, then warmed to 50° C. and was stirred at that temperature for 1 h. The reaction was then cooled and placed in the freezer overnight. The precipitates were filtered by gravity, washing with carbon tetrachloride. The solvent was removed in vacuo, with low heat, to yield 58 g of the desired benzyl 2-chloro-2-[(chlorocarbonyl)oxy]acetate as a clear colorless oil. ¹H-NMR (300 MHz, CDCl₃) δ (ppm): 5.24 (s, 2H), 6.45 (s, 1H), 7.32 (s, 5H).

### EXAMPLE 2

Ethyl 2-chloro-2-[(chlorocarbonyl)oxy]Acetate (Compound 1-2 of Table 1)

Pyridine (0.145 mL, 1.79 mmol) was added to a solution of polymeric ethyl glyoxylate (18.51 g, 181 mmol) and triphosgene (48.5 g, 163 mmol) in dry THF at room temperature in a flask fitted with a reflux condenser and connected to a N₂ bubbler. After 10 min the flask was placed in a pre-heated 65° C. oil bath. After 21 h, the reaction was allowed to cool to room temperature, and then the mixture was concentrated under vacuum. Ether was added to the residue, the mixture was filtered through Celite, and the filtrate was concentrated, affording 34.6 g (87% yield) of a yellow oil. ¹H-NMR (300 MHz, CDCl₃) δ (ppm): 1.36 (t, 3H) 4.36 (q, 2H), 6.48 (s, 1H).

### EXAMPLE 3

Methyl 2-chloro-2-[(chlorocarbonyl)oxy]Acetate (Compound 1-3 of Table 1)

The title compound was prepared according to the procedure described in Example 2 above, except methyl glyoxylate was substituted for ethyl glyoxylate. ¹H-NMR (300 MHz, CDCl₃) δ (ppm): 3.92 (s, 3H), 6.52 (s, 1H).

### EXAMPLE 4

Isopropyl 2-chloro-2-[(chlorocarbonyl)oxy]Acetate (Compound 1-⁴ of Table 1)

The title compound was prepared according to the procedure described in Example 2 above, except isopropyl glyoxylate was substituted for ethyl glyoxylate. ¹H-NMR (300 MHz, CDCl₃) δ (ppm): 1.33 (s, 3H), 1.35 (s, 3H), 5.2 (q, 1H), 6.44 (s, 1H).

### EXAMPLE 5

Tert-Butyl 2-chloro-2-[(chlorocarbonyl)oxy]Acetate (Compound 1-5 of Table 1)

The title compound was prepared according to the procedure described in Example 2 above, except tert-butyl glyoxylate was substituted for ethyl glyoxylate. ¹H-NMR (300 MHz, CDCl₃) δ (ppm): 1.53 (s, 9H), 6.35 (s, 1H).

### EXAMPLE 6

N-Butyl 2-chloro-2-[(chlorocarbonyl)oxy]Acetate (Compound 1-6 of Table 1)

The title compound was prepared according to the procedure described in Example 2 above, except n-butyl glyoxylate was substituted for ethyl glyoxylate. ¹H-NMR (300 MHz, CDCl³) δ (ppm): 0.94 (t, ³H), 1.40 (q, 2H), 1.68 (m, 2H), 4.28 (m, 2H), 6.49 (s, 1H).

Following the general methods described hereinbefore, the following compounds of Formula III as listed in Table 2 were prepared.

TABLE 2

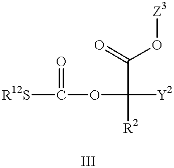

Listing of Compounds of Formula III

| Cmpd# | Y¹ | Y² | R² | Z³ |
|---|---|---|---|---|
| 2-2 | Et | H | Cl | ethyl |
| 2-2 | Et | H | I | ethyl |
| 2-3 | Et | H | Cl | n-butyl |
| 2-4 | Et | H | I | n-butyl |
| 2-5 | Et | H | Cl | isopropyl |
| 2-6 | Et | H | I | isopropyl |

The following Examples are provided for guidance to the practitioner in order to practice the invention.

### EXAMPLE 7

Chloro-ethylsulfanylcarbonyloxy-acetic Acid Ethyl Ester (Compound 2-1 of Table 2)

1000 mL round bottom flask was charged with sodium ethylthiolate (13.3 g, 158 mmol) and 500 mL of dry diethyl ether. The mixture was cooled to −70° C. in an acetone dry ice bath. Ethyl 2-chloro-2-[(chlorocarbonyl)oxy]acetate (32.8 g, 155 mmol) was added as a solution in 20 mL of diethyl ether over 1.5 h at such a rate that the reaction temperature did not exceed −65° C. The reaction was allowed to warm to room temperature and stir for 16 h. The reaction was vacuum filtered, the filtrate dried (MgSO₄), gravity filtered, and concentrated under reduced pressure to yield 33.5 g of a clear liquid. ¹H-NMR (300 MHz, CDCl₃) δ (ppm): 1.33 (m, 6H), 2.94 (q, 2H), 4.31 (q, 2H), 6.65 (s, 1H).

### EXAMPLE 8

Iodo-ethylsulfanylcarbonyloxy-acetic Acid Ethyl Ester (Compound 2-2 of Table 2)

To a stirred solution of chloro-ethylsulfanylcarbonyloxy-acetic acid ethyl ester (33.5 g, 148 mmol) in 160 mL of dry acetone was added NaI (28.8. g, 192 mmol). The mixture was stirred at room temperature for 4 h. The acetone was removed and the remaining slurry was diluted with 100 mL of diethyl ether. The mixture was filtered through Celite and concentrated under reduced pressure to yield a brown liquid. The liquid was redissolved in 50 mL of diethyl ether and gravity filtered to afford 37.1 g of a brown liquid. ¹H-NMR (300 MHz, CDCl₃) δ (ppm): 1.33 (m, 6H), 2.95 (q, 2H), 4.30 (q, 2H), 7.21 (s, 1H).

### EXAMPLE 9

Chloro-ethylsulfanylcarbonyloxy-acetic Acid Butyl Ester (Compound 2-3 of Table 2)

The title compound was prepared according to the procedure described in Example 7 above except for the substitution of n-butyl 2-chloro-2-[(chlorocarbonyl)oxy] acetate for ethyl 2-chloro-2 -[(chlorocarbonyl)oxy] acetate. ¹H-NMR (300 MHz, CDCl₃) δ (ppm): 0.95 (t, 3H), 1.33-1.43 (m, 5H), 1.67-1.72 (m, 2H), 2.93 (q, 2H), 4.25-4.30 (m, 2H), 6.65 (s, 1H).

### EXAMPLE 10

Iodo-ethylsulfanylcarbonyloxy-acetic Acid Butyl Ester (Compound 2-⁴ of Table 2)

The title compound was prepared according to the procedure described in Example 8 above except for the substitution of chloro-ethylsulfanylcarbonyloxyacetic acid butyl ester for chloro-ethylsulfanylcarbonyloxy-acetic acid ethyl ester. ¹H-NMR (300 MHz, CDCl₃) δ (ppm): 0.95 (t, 3H), 1.32-1.43 (m, 5H), 1.65-1.70 (m, 2H), 2.92-2.95 (m, 2H), 4.22-4.26 (m, 2H), 7.21 (s, 1H).

### EXAMPLE 11

Chloro-ethylsulfanylcarbonyloxy-acetic Acid Isopropyl Ester (Compound 2-5 of Table 2)

The title compound was prepared according to the procedure described in Example 7 above except for the substitution of isopropyl 2-chloro-2-[(chlorocarbonyl)oxy] acetate for ethyl 2-chloro-2-[(chlorocarbonyl)oxy] acetate. ¹H-NMR (300 MHz, CDCl₃) δ (ppm): 1.30 (t, 9H), 2.90 (q, 2H), 5.10 (s, 1H), 6.60 (s, 1H).

### EXAMPLE 12

Iodo-ethylsulfanylcarbonyloxy-acetic Acid Isopropyl Ester (Compound 2-6 of Table 2)

The title compound was prepared according to the procedure described in Example 8 above except for the substitution of chloro-ethylsulfanylcarbonyloxy- acetic acid isopropyl ester for chloro-ethylsulfanylcarbonyloxy-acetic acid ethyl ester. ¹H-NMR (300 MHz, CDCl₃) δ (ppm): 1.30 (t, 9H), 2.90 (q, 2H), 5.10 (s, 1H), 7.15 (s, 1H).

Following the general methods described hereinbefore, the following compounds of Formula II as listed in Table 3 were prepared.

TABLE 3

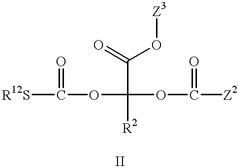

Listing of Compounds of Formula II

| Cmpd# | R¹² | R² | Z³ | Z² |
|---|---|---|---|---|
| 3-1 | Et | H | ethyl | 2-propyl |
| 3-2 | Et | H | ethyl | tert-butyl |
| 3-3 | Et | H | ethyl | ethyl |
| 3-4 | Et | H | ethyl | 2-ethoxyphenyl |
| 3-5 | Et | H | ethyl | phenyl |
| 3-6 | Et | H | ethyl | 2,4-dichlorophenoxymethyl |
| 3-7 | Et | H | ethyl | 3-(2,4-dichlorophenoxy)propyl |
| 3-8 | Et | H | ethyl | 1-(2,4-dichlorophenoxy)ethyl |
| 3-9 | Et | H | ethyl | 2,5-dichloro-6-methoxyphenyl |
| 3-10 | Et | H | ethyl | 2,4,6-trimethylphenyl |
| 3-11 | Et | H | 2-propyl | phenyl |
| 3-12 | Et | H | 2-propyl | tert-butyl |
| 3-13 | Et | H | 2-propyl | 1-methyl-1-cyclopropyl |
| 3-14 | Et | H | 2-propyl | 2-propyl |
| 3-15 | Et | H | 2-propyl | ethyl |
| 3-16 | Et | H | 2-propyl | N-acetyl-N-methyl-aminomethyl |
| 3-17 | Et | H | n-butyl | (diethoxyphosphoryl)methyl |
| 3-18 | Et | H | n-butyl | tert-butyl |
| 3-19 | Et | H | 2-propyl | 3,7-dichloro-8-quinoline |

The following Examples are provided for guidance to the practitioner in order to practice the invention.

### EXAMPLE 13

2-Methylpropanoic Acid Ethoxycarbonyl-ethylsulfanylcarbonyloxy-methyl Ester (Compound ³-1 of Table ³)

To a stirred ice cold solution of iodo-ethylsulfanylcarbonyloxy-acetic acid ethyl ester (5.4 g, 17.0 mmol) in 20 mL of dry THF was added 2-methylpropanoic acid (1.94 g, 22.1 mmol) followed by diisopropylethylamine (DIEA) (2.85 g, 22.1 mmol). The reaction was allowed to stir at room temperature for 16 h. The reaction was diluted with 100 mL of diethyl ether, gravity filtered, and concentrated under reduced pressure. The liquid was suction filtered through a pad of flash grade silica gel and eluted with 20% methylene chloride/hexanes. ¹H-NMR (300 MHz, CDCl₃) δ (ppm): 1.24 (m, 6H), 1.33 (m, 6H), 2.66 (m, 1H), 2.90 (q, 2H), 4.28 (q, 2H), 5.92 (s, 1H).

### EXAMPLE 14

Pivalic Acid ethoxycarbonyl-ethylsulfanylcarbonyloxy-methyl Ester (Compound 3-2 of Table 3)

The title compound was prepared according to the procedure described in Example 13 above except for the substitution of pivalic acid for 2-methylpropanoic acid. 1H-NMR (300 MHz, CDCl₃) δ (ppm): 1.25 (s, 9H), 1.33 (m, 6H), 2.88 (q, 2H), 4.28 (q, 2H), 6.82 (s, 1H).

### EXAMPLE 15

Propionic Acid ethoxycarbonyl-ethylsulfanylcarbonyloxy-methyl Ester (Compound 3-3 of Table 3)

The title compound was prepared according to the procedure described in Example 13 above except for the substitution of propionic acid for 2-methylpropanoic acid. ¹H-NMR (300 MHz, CDCl₃) δ (ppm): 1.16 (t, 3H), 1.30 (m, 6H), 2.47 (q, 2H), 2.93 (q, 2H), 4.29 (q, 2H), 6.94 (s, 1H).

### EXAMPLE 16

2-Ethoxybenzoic acid ethoxycarbonyl-ethylsulfanylcarbonyloxy-methyl Ester (Compound 3-4 of Table 3)

The title compound was prepared according to the procedure described in Example 13 above except for the substitution of 2-ethoxybenzoic acid for 2-methylpropanoic acid. 1H-NMR (300 MHz, CDCl₃) δ (ppm): 1.23 (m, 6H), 1.44 (t, 3H), 2.92 (q, 2H), 4.12 (q, 2H), 4.31 (q, 2H), 6.96 (m, 2H), 7.15 (s, 1H), 7.43 (t, 1H), 7.92 (d, 1H).

### EXAMPLE 17

Benzoic Acid ethoxycarbonyl-ethylsulfanylcarbonyloxy-methyl Ester (Compound 3-5 of Table 3)

The title compound was prepared according to the procedure described in Example 13 above except for the substitution of benzoic acid for 2-methylpropanoic acid. 1H-NMR (300 MHz, CDCl₃) δ (ppm): 1.32 (m, 6H), 2.93 (q, 2H), 4.35 (q, 2H), 7.19 (s, 1H), 7.35 (t, 2H), 7.48 (t, 1H), 8.10 (d, 2H).

### EXAMPLE 18

2,4-Dichlorophenoxyacetic Acid Ethoxycarbonyl-ethylsulfanylcarbonyloxy-methyl Ester (Compound 3-6 of Table 3)

The title compound was prepared according to the procedure described in Example 13 above except for the substitution of 2,4-dichlorophenoxyacetic acid for 2-methylpropanoic acid. ¹H-NMR (300 MHz, CDCl₃) δ (ppm): 1.32 (m, 6H), 2.92 (q, 2H), 4.29 (q, 2H), 4.82 (s, 2H), 6.83 (d, 1H), 6.95 (s, 1H), 7.24 (dd, 1H), 7.38 (s, 1H).

### EXAMPLE 19

4-(2,4-Dichlorophenoxy)butyric Acid Ethoxycarbonyl-ethylsulfanylcarbonyloxymethyl Ester (Compound 3-7 of Table 3)

The title compound was prepared according to the procedure described in Example 13 above except for the substitution of 4-(2,4-diclorophenoxy)butyric acid for 2-methylpropanoic acid. 1H-NMR (300 MHz, CDCl₃) δ (ppm): 1.32 (m, 6H), 2.18 (m, 2H), 2.76 (t, 2H), 2.97 (q, 2H), 4.02 (q, 2H), 4.31 (q, 2H), 6.83 (d, 1H), 6.86 (s, 1H), 7.15 (d, 1H), 7.35 (d, 1H).

### EXAMPLE 20

2′-(2,4-Dichlorophenoxy)propionic Acid Ethoxycarbonyl-ethylsulfanylcarbonyloxymethyl Ester (Compound 3-8 of Table 3)

The title compound was prepared according to the procedure described in Example 13 above except for the substitution of 2′-(2,4-diclorophenoxy)propionic acid for 2-methylpropanoic acid. ¹H-NMR (300 MHz, CDCl₃) δ (ppm): 1.32 (m, 6H), 1.74 (m, 3H), 2.91 (q, 2H), 4.26 (q, 2H), 4.88 (m, 1H), 6.88 (m, 1H), 6.95 (s, 1H), 7.18 (d, 1H), 7.38 (s, 1H).

### EXAMPLE 21

2,5-Dichloro-6-methoxybenzoic Acid Ethoxycarbonyl-ethylsulfanylcarbonyloxymethyl Ester (Compound 3-9 of Table 3)

The title compound was prepared according to the procedure described in Example 13 above except for the substitution of 2,5-dichloro-6-methoxybenzoic acid for 2-methylpropanoic acid. ¹H-NMR (300 MHz, CDCl₃) δ (ppm): 1.34 (m, 6H), 2.93 (q, 2H), 3.94 (s, 3H), 4.34 (q, 2H), 7.16 (d, 1H), 7.18 (s, 1H), 7.39 (d, 1H).

### EXAMPLE 22

2,4,6-Trimethylbenzoic Acid Ethoxycarbonyl-ethylsulfanylcarbonyloxy-methyl Ester (Compound 3-10 of Table 3)

The title compound was prepared according to the procedure described in Example 13 above except for the substitution of 2,4,6-trimethylbenzoic acid for 2-methylpropanoic acid. ¹H-NMR (300 MHz, CDCl₃) δ (ppm): 1.33 (m, 6H), 2.27 (s, 3H), 2.34 (s, 6H), 2.90 (q, 2H), 4.31 (q, 2H), 6.86 (s, 2H), 7.16 (s, 1H).

### EXAMPLE 23

Benzoic Acid Isopropoxycarbonyl-ethylsulfanylcarbonyloxy-methyl Ester (Compound 3-11 of Table 3)

To a stirred ice cold solution of iodo-ethylsulfanylcarbonyloxy-acetic acid isopropyl ester (3.1 g, 9.3 mmol) in 40 mL of dry THF was added benzoic acid (1.5 g, 12.0 mmol) followed by DIEA (2.1 mL, 12.0 mmol). The reaction was allowed to stir at room temperature for 16 h. The solvent was removed under reduced pressure. The residue was dissolved in ether and washed 3 times with a saturated solution of sodium bicarbonate and once with brine. The ether layer was dried over MgSO₄ and concentrated to yield 2.0 g (66%) of the desired product, which was used without further purification. ¹H-NMR (300 MHz, CDCl₃) δ (ppm): 1.30 (m, 9H), 2.90 (q, 2H), 5.15 (m, 1H), 7.13 (s, 1H), 7.55 (m, 3H), 8.10 (m, 2H).

### EXAMPLE 24

Pivalic Acid Isopropoxycarbonyl-ethyl Sulfanylcarbonyloxy-methyl Ester (Compound 3-12 of Table 3)

The title compound was prepared according to the procedure described in Example 23 above except for the substitution of pivalic acid for benzoic acid. ¹H-NMR (300 MHz, CDCl₃) δ (ppm): 1.30 (m, 18H), 2.90 (q, 2H), 5.10 (m, 1H), 6.85 (s, 1H).

### EXAMPLE 25

1-Methyl-1-cyclopropanecarboxylic Acid Isopropoxycarbonylethylsulfanylcarbonyloxy-methyl Ester (Compound 3-13 of Table 3)

The title compound was prepared according to the procedure described in Example 23 above except for the substitution of 1-methyl-1-cyclopropanecarboxylic acid for benzoic acid. ¹H-NMR (300 MHz, CDCl₃) δ (ppm): 0.75 (d, 2H), 1.35 (m, 14H), 2.90 (m, 2H), 5.10 (m, 1H), 6.85 (s, 1H).

### EXAMPLE 26

Isobutyric Acid Isopropoxycarbonyl-ethylsulfanylcarbonyloxy-methyl Ester (Compound 3-14 of Table 3)

The title compound was prepared according to the procedure described in Example 23 above except for the substitution of isobutyric acid for benzoic acid. ¹H-NMR (300 MHz, CDCl₃) δ (ppm): 1.25 (m, 15H), 2.65 (m, 1H), 2.90 (m, 2H), 5.10 (m, 1H), 6.90 (s, 1H).

### EXAMPLE 27

Propionic Acid isopropoxycarbonyl-ethylsulfanylcarbonyloxy-methyl Ester (Compound 3-15 of Table 3)

The title compound was prepared according to the procedure described in Example 23 above except for the substitution of propionic acid for benzoic acid. ¹H-NMR (300 MHz, CDCl₃) δ (ppm): 1.15 (t, 3H), 1.30 (m, 9H), 2.45 (q, 2H), 2.90 (q, 2H), 5.10 (m, 1H), 6.90 (s, 1H).

### EXAMPLE 28

Acetyl Methyl Carbamic Acid Isopropoxycarbonyl-ethylsulfanylcarbonyloxymethyl Ester (Compound 3-16 of Table 3)

The title compound was prepared according to the procedure described in Example 23 above except for the substitution of acetyl methyl carbamic acid for benzoic acid. ¹H-NMR (300 MHz, CDCl₃) δ (ppm): 1.30 (m, 9H), 2.10 (d, 3H), 2.90 (m, 2H), 3.10 (d, 3H), 4.20 (m, 2H), 5.10 (m, 1H), 6.90 (s, 1H).

### EXAMPLE 29

Diethylphosphonoacetic Acid Butoxycarbonyl-ethylsulfanylcarbonyloxy-methyl Ester (Compound 3-17 of Table 3)

The title compound was prepared according to the procedure described in Example 13 above except for the substitution of diethylphosphonoacetic acid for 2-methylpropanoic acid and iodoethylsulfanylcarbonyloxy-acetic acid butyl ester for iodoethylsulfanylcarbonyloxy-acetic acid ethyl ester. ¹H-NMR (300 MHz, CD₃OD) δ (ppm): 1.35 (t, 6H), 2.89 (q, 2H), 3.03 (d, 2H), 4.18 (q, 4H), 5.84 (s, 2H).

### EXAMPLE 30

Pivalic Acid Butoxycarbonyl-ethylsulfanylcarbonyloxy-methyl Ester (Compound 3-18 of Table 3)

The title compound was prepared according to the procedure described in Example 13 above except for the substitution of pivalic acid for 2-methylpropanoic acid and iodoethylsulfanylcarbonyloxy-acetic acid butyl ester for iodoethylsulfanylcarbonyloxy-acetic acid ethyl ester. ¹H-NMR (300 MHz, CD₃OD) δ (ppm): 1.25 (s, 9H), 1.31-1.45 (m, 8H), 1.65 (m 2H), 2.95 (q, 2H), 4.25 (m, 2H), 6.92 (s, 1H).

### EXAMPLE 31

3,7-Dichloro-8-quinolinecarboxylic Acid Isopropoxycarbonylethylsulfanylcarbonyloxy-methyl Ester (Compound 3-19 of Table 3)

The title compound was prepared according to the procedure described in Example 23 above except for the substitution of 3,7-dichloro-8-quinolinecarboxylic acid for benzoic acid. ¹H-NMR (300 MHz, CDCl₃) d (ppm): 1.35 (m, 9H), 2.95 (q, 2H), 5.15 (m, 1H), 7.25 (s, 1H), 7.57 (d, 1H), 7.76 (d, 1H), 8.14 (s, 1H) 8.83 (s, 1H).

Following the general methods described hereinbefore, the following compounds of Formula I as listed in Table 4 were prepared.

TABLE 4

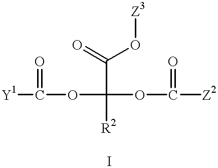

Listing of Compounds of Formula I

| Cmpd# | Y¹ | R² | Z³ | Z² |
|---|---|---|---|---|
| 4-1 | Cl | H | ethyl | 2-propyl |
| 4-2 | Cl | H | ethyl | tert-butyl |
| 4-3 | Cl | H | ethyl | ethyl |
| 4-4 | Cl | H | ethyl | 2-ethoxyphenyl |
| 4-5 | Cl | H | ethyl | phenyl |
| 4-6 | Cl | H | ethyl | 3-(2,4-dichlorophenoxy)propyl |
| 4-7 | Cl | H | ethyl | 1-(2,4-dichlorophenoxy)ethyl |
| 4-8 | Cl | H | ethyl | 2,5-dichloro-6-methoxyphenyl |
| 4-9 | Cl | H | ethyl | 2,4,6-trimethylphenyl |
| 4-10 | Cl | H | n-butyl | diethylphosphonomethyl |
| 4-11 | Cl | H | n-butyl | tert-butyl |
| 4-12 | Cl | H | 2-propyl | 2-propyl |

The following Examples are provided for guidance to the practitioner in order to practice the invention.

### EXAMPLE 32

2-Methylpropionic Acid Ethoxycarbonyl-chlorocarbonyloxy-methyl Ester (Compound 4-1 of Table 4)

A 100 mL round bottom flask was charged with 2-methylpropionic acid ethoxycarbonyl-ethyl sulfanylcarbonyloxy-methyl ester (4.4 g, 15.8 mmol) and cooled to 5° C. Sulfuryl chloride (2.70 g, 20.0 mmol) was added over 1 min. After 30 min of stirring, the cooling bath was removed and the reaction was allowed to stir for 3 h at room temperature and then placed under vacuum. The material was used without purification. ¹H-NMR (300 MHz, CDCl₃) δ (ppm): 1.26 (m, 9H), 2.58 (m, 1H), 4.32 (q, 2H), 6.83 (s, 1H).

### EXAMPLE 33

Pivalic Acid Ethoxycarbonyl-chlorocarbonyloxy Methyl Ester (Compound 4-2 of Table 4)

The title compound was prepared according to the procedure described in Example 32 above except for the substitution of pivalic acid ethoxycarbonyl-ethylsulfanylcarbonyloxy methyl ester for isobutyric acid ethoxycarbonyl-ethylsulfanylcarbonyloxy-methyl ester. ¹H-NMR (300 MHz, CDCl₃) δ (ppm): 1.25 (s, 9H), 1.33 (t, 3H), 4.32 (q, 2H), 6.78 (s, 1H).

### EXAMPLE 34

Propionic Acid Ethoxycarbonyl-chlorocarbonyloxy-methyl Ester (Compound 4-3 of Table 4)

The title compound was prepared according to the procedure described in Example 32 above except for the substitution propionic acid ethoxycarbonyl-ethylsulfanylcarbonyloxy-methyl ester for isobutyric acid ethoxycarbonyl-ethylsulfanylcarbonyloxy-methyl ester, using 1.4 equiv of sulfuryl chloride and starting the reaction at 5° C. ¹H-NMR (300 MHz, CDCl₃) δ (ppm): 1.21 (t, 3H), 1.35 (t, 3H), 2.53 (q,2H), 4.32 (q, 2H), 6.84 (s, 1H).

### EXAMPLE 35

2-Ethoxybenzoic Acid Ethoxycarbonyl-chlorocarbonyloxy-methyl Ester (Compound 4-4 of Table 4)

The title compound was prepared according to the procedure described in Example 32 above except for the substitution 2-ethoxybenzoic acid ethoxycarbonylethylsulfanyl-carbonyloxy methyl ester for isobutyric acid ethoxycarbonyl-ethylsulfanylcarbonyloxy-methyl ester, using 1.4 equiv of sulfuryl chloride and starting the reaction at 5° C. 1H-NMR (300 MHz, CDCl₃) δ (ppm): 1.36 (t, 3H), 1.45 (t, 3H), 4.13 (q, 2H), 4.36 (q, 2H), 6.95 (m, 2H), 7.02 (s, 1H), 7.47 (t, 1H), 7.91(d, 1H).

### EXAMPLE 36

Benzoic Acid Ethoxycarbonyl-chlorocarbonyloxy-methyl Ester (Compound 4-5 of Table 4)

The title compound was prepared according to the procedure described in Example 32 above except for the substitution benzoic acid ethoxycarbonyl-ethylsulfanylcarbonyloxy-methyl ester for isobutyric acid ethoxycarbonyl-ethylsulfanylcarbonyloxy-methyl ester, using 1.2 equiv of sulfuryl chloride and starting the reaction at 5° C. ¹H-NMR (300 MHz, CDCl₃) δ (ppm): 1.36 (t, 3H), 4.32 (q, 2H), 7.09 (s, 1H), 7.50 (t, 2H), 7.63 (t, 1H), 8.10 (d, 2H).

### EXAMPLE 37

2,4-Dichlorophenoxybutyric Acid Ethoxycarbonyl-chlorocarbonyloxy-methyl Ester (Compound 4-6 of Table 4)

The title compound was prepared according to the procedure described in Example 32 above except for the substitution 2,4-dichlorophenoxybutyric acid ethoxycarbonylethyl-sulfanylcarbonyloxy-methyl ester for isobutyric acid ethoxycarbonyl ethylsulfanylcarbonyl-oxy-methyl ester, using 1.45 equiv of sulfuryl chloride and starting the reaction at 5° C. ¹H-NMR (300 MHz, CDCl₃) δ (ppm): 1.34 (t, 3H), 2.21 (m, 2H), 2.56 (t, 2H), 4.07 (t, 2H), 4.31 (q, 2H), 6.83 (d, 1H), 6.86 (s, 1H), 7.17 (d, 1H), 7.35 (d, 1H).

### EXAMPLE 38

2′-(2,4-Dichlorophenoxy)propionic Acid Ethoxycarbonyl-chlorocarbonyloxy-methyl Ester (Compound 4-7 of Table 4)

The title compound was prepared according to the procedure described in Example 32 above except for the substitution 2′-(2,4-dichlorophenoxy)propionic acid ethoxycarbonyl-ethylsulfanylcarbonyloxy-methyl ester for isobutyric acid ethoxycarbonyl-ethylsulfanylcarbonyloxy-methyl ester, using 1.80 equiv of sulfuryl chloride and starting the reaction at 5° C. ¹H-NMR (300 MHz, CDCl₃) δ (ppm): 1.37 (t, 3H), 1.92 (m, 3H), 4.33 (q, 2H), 4.83 (m, 1H), 6.84 (m, 2H), 7.16 (dd, 1H), 7.38 (d, 1H).

### EXAMPLE 39

2,5-Dichloro-6-methoxybenzoic Acid Ethoxycarbonyl-chlorocarbonyloxy-methyl Ester (Compound 4-8 of Table 4)

The title compound was prepared according to the procedure described in Example 32 above except for the substitution 2,5-dichloro-6-methoxybenzoic acid ethoxycarbonyl-ethylsulfanylcarbonyloxy-methyl ester for isobutyric acid ethoxycarbonyl-ethylsulfanylcarbonyloxy-methyl ester, using 1.45 equiv of sulfuryl chloride and starting the reaction at 5° C. ¹H-NMR (300 MHz, CDCl₃) δ (ppm): 1.34 (t, 3H), 3.95 (s, 3H), 4.36 (q, 2H), 7.08 (s, 1H), 7.18 (d, 1H), 7.42 (d, 1H).

### EXAMPLE 40

2,4,6-Trimethylbenzoic Acid Ethoxycarbonyl-chlorocarbonyloxy-methyl Ester (Compound 4-9 of Table 4)

The title compound was prepared according to the procedure described in Example 32 above except for the substitution of 2,4,6-trimethylbenzoic acid ethoxycarbonyl-ethylsulfanylcarbonyloxy-methyl ester for isobutyric acid ethoxycarbonyl-ethylsulfanylcarbonyloxy-methyl ester, using 1.45 equiv of sulfuryl chloride and starting the reaction at 5° C. ¹H-NMR (300 MHz, CDCl₃) δ (ppm): 1.34 (t, 3H), 2.28 (s, 3H), 2.35 (s, 6H), 4.34 (q, 2H), 6.89 (s, 1H), 7.06 (s, 1H), 7.12 (s, 1H).

### EXAMPLE 41

Diethylphosphonoacetic Acid Butoxycarbonyl-chlorocarbonyloxy-methyl Ester (Compound 4-10 of Table 4)

The title compound was prepared according to the procedure described in Example 32 above except for the substitution diethylphosphonoacetic acid butoxycarbonyl-ethylsulfanylcarbonyloxy-methyl ester for isobutyric acid ethoxycarbonyl-ethylsulfanylcarbonyloxy-methyl ester, using 1.9 equiv of sulfuryl chloride and starting the reaction at 5° C. ¹H-NMR (300 MHz, CDCl₃) δ (ppm): 0.96 (t, 3H), 1.37 (t, 6H), 1.72 (m, 2H), 2.01 (m, 2H), 3.15 (d, 2H), 4.27 (m, 6H), 6.85 (s, 1H).

### EXAMPLE 42-A

Pivalic Acid Butoxycarbonyl-chlorocarbonyloxy-methyl Ester (Compound 4-11 of Table 4)

The title compound was prepared according to the procedure described in Example 32 above except for the substitution of pivalic acid butoxycarbonyl-ethylsulfanylcarbonyloxy-methyl ester for isobutyric acid ethoxycarbonyl-ethylsulfanylcarbonyloxy-methyl ester, using 1.9 equiv of sulfuryl chloride and starting the reaction at 5° C. ¹H-NMR (300 MHz, CDCl₃) δ (ppm): 0.95 (t, 3H), 1.27 (s, 9H), 1.35-1.45 (m, 2H), 1.65-1.75 (m 2H), 4.31 (m, 2H), 6.85 (s, 1H).

### EXAMPLE 42-B

2-Methylpropanoic Acid (Chlorocarbonyloxy)-[(1-methylethoxy)carbonyl]Methyl Ester Compound 4-12 of Table 4)

The title compound was prepared according to the procedure described in Example 32 above except for the substitution of isobutyric acid isobutoxycarbonyl-ethylsulfanylcarbonyloxy-methyl ester for isobutyric acid ethoxycarbonyl-ethylsulfanylcarbonyloxy-methyl ester, using 1.9 equiv of sulfuryl chloride and starting the reaction at 5° C. ¹H-NMR (300 MHz, CDCl₃) δ (ppm): 1.22-1.30 (m, 6H), 1.30-1.40 (m, 6H), 2.70 (heptet, 1H), 5.14 (heptet, 1H), 6.77 (s, 1H).

We claim:

1. A process leading to a compound of formula (V) 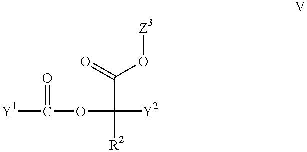
comprising the step of reacting, in the presence of a suitable solvent and a suitable catalyst, a compound of formula (VII) 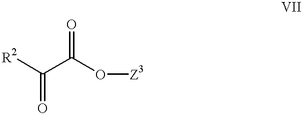
with a compound of formula (VIII) 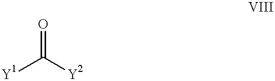
wherein
Z³ is alkyl, alkylcarbonyloxyalkyl, hydroxyalkyl, alkylsulfonylalkyl, acetylaminoalkyl, haloalkyl, alkenyl, acetylaminoalkenyl, haloalkenyl, alkynyl, haloalkynyl, cycloalkyl, cycloalkenyl, carboxycycloalkyl, carboxycycloalkenyl, cycloalkylalkyl, cycloalkylalkenyl, cycloalkenylalkyl, cycloalkenylalkenyl, cycloalkylalkynyl, cycloalkenylalkynyl, carboxycyclo alkylalkyl, carboxycycloalkylalkenyl, carboxycycloalkenylalkyl, carboxycycloalkenylalkenyl, carboxycycloalkylalkynyl, carboxycycloalkenylalkynyl, heterocyclyl, heterocyclylalkyl, heterocyclylalkenyl, heterocyclylalkynyl, alkoxyalkyl, alkoxyalkoxyalkyl, alkoxyalkenyl, alkoxyalkynyl, alkoxycarbonylalkyl, alkoxycarbonylalkenyl, alkoxycarbonylalkynyl, haloalkoxyalkyl, haloalkoxyalkenyl, haloalkoxyalkynyl, alkylthioalkyl, alkylthlioalkenyl, alkylthioalkynyl, haloalkylthioalkyl, haloalkylthioalkenyl, haloalkylthioalkynyl, carboxyalkyl, carboxyalkenyl, carboxyalkynyl, SiR³R⁴R⁵, aryl, aryl substituted with one or more substituents independently selected from halo, nitro, hydroxy, cyano, thiocyanato, alkyl, alkylsulfonylalkyl, alkenyl, alkynyl, haloalkyl, haloalkenyl, haloalkynyl, alkoxy, haloalkoxy, SO₂NR³R⁴ and NR³R⁴, aralkyl, aralkenyl, aralkynyl, arcycloalkyl, aroxyalkyl, or aralkyl, aralkenyl, aralkynyl, arcycloalkyl, aroxyalkyl substituted with one or more substituents independently selected from halo, nitro, hydroxy, cyano, alkyl, cycloalkyl, alkenyl, alkynyl, haloalkyl, haloalkenyl, haloalkynyl, alkoxy, haloalkoxy, SO₂NR³R⁴ and NR³R⁴, heteroaryl, heteroaryl substituted with one or more substituents independently selected from halo, nitro, hydroxy, cyano, alkyl, alkenyl, alkynyl, haloalkyl, haloalkenyl, haloalkynyl, alkoxy, haloalkoxy, SO₂NR³R⁴ and NR³R⁴, heteroaralkyl, heteroaralkenyl, heteroaralkynyl, or heteroaralkyl, heteroaralkenyl, heteroaralkynyl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴,
R² is a hydrogen atom, alkyl, alkenyl, alkynyl, alkoxyalkyl, alkoxyalkenyl, alkoxyalkynyl, alkylthioalkyl, alkylthioalkenyl, alkylthioalkynyl, carboxy, a carboxylate salt, carboxyalkyl, carboxyalkenyl, carboxyalkynyl, alkoxycarbonyl, alkoxycarbonylalkyl, alkoxycarbonylalkenyl, alkoxycarbonylalkynyl, alkylcarbonyl, alkenylcarbonyl, alkynylcarbonyl, cycloalkyl, cycloalkenyl, cycloalkylalkyl, cycloalkenylalkyl, cycloalkylalkenyl, cycloalkenylalkenyl, cycloalkylalkynyl, cycloalkenylalkynyl, heterocyclyl, heterocyclylalkyl, heterocyclylalkenyl, heterocyclylalkynyl, or alkyl, alkenyl, alkynyl, alkoxyalkyl, alkoxyalkenyl, alkoxyalkynyl, alkylthioalkyl, alkylthioalkenyl, alkylthioalkynyl, carboxyalkyl, carboxyalkenyl, carboxyalkynyl, alkoxycarbonyl, alkoxycarbonylalkyl, alkoxycarbonylalkenyl, alkoxycarbonylalkynyl, alkylcarbonyl, alkenylcarbonyl, alkynylcarbonyl, cycloalkyl, cycloalkenyl, cycloalkylalkyl, cycloalkenylalkyl, cycloalkylalkenyl, cycloalkenylalkenyl, cycloalkylalkynyl, cycloalkenylalkynyl, heterocyclyl, heterocyclylalkyl, heterocyclylalkenyl, heterocyclylalkynyl substituted with one or more substituents independently selected from halo, cyano, hydroxy, nitro, SO₂NR³R⁴ and NR³R⁴, aryl or aryl substituted with one or more substituents independently selected from halo, hydroxy, cyano, nitro, alkyl, alkenyl, alkynyl, haloalkyl, haloalkenyl, haloalkynyl, alkoxy, haloalkoxy, carboxy, alkoxycarbonyl, SO₂NR³R⁴ and NR³R⁴, aralkyl, aralkenyl, aralkynyl or aralkyl, aralkenyl, aralkynyl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, arylcarbonyl, aralkylcarbonyl, aralkenylcarbonyl, aralkynylcarbonyl or arylcarbonyl, aralkylcarbonyl, aralkenylcarbonyl, aralkynylcarbonyl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, heteroaryl or heteroaryl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, heteroaralkyl, heteroaralkenyl, heteroaralkynyl or heteroaralkyl, heteroaralkenyl, heteroaralkynyl substituted with one or more substituents independently selected from halo, cyano, hydroxy, nitro, alkyl, alkenyl, alkynyl, haloalkyl, haloalkenyl, haloalkynyl, alkoxy, haloalkoxy, SO₂NR³R⁴ and NR³R⁴, heteroaralkylcarbonyl, heteroaralkenylcarbonyl, heteroaralkynylcarbonyl or heteroaralkylcarbonyl, heteroaralkenylcarbonyl, heteroaralkynylcarbonyl substituted with one or more substituents independently selected from halo, cyano, hydroxy, nitro, alkyl, alkenyl, alkynyl, haloalkyl, haloalkenyl, haloalkynyl, alkoxy, haloalkoxy, SO₂NR³R⁴ and NR³R⁴,
R³, R⁴ and R⁵ are each independently a hydrogen atom, alkyl, cycloalkyl, cycloalkenyl, cycloalkylalkyl, cycloalkylalkenyl, cycloalkylalkynyl, cycloalkenylalkyl, cycloalkenylalkenyl, cycloalkenylalkynyl, carboxyalkyl, carboxyalkenyl, carboxyalkynyl, heterocyclyl, heterocyclylalkyl, heterocyclylalkenyl, heterocyclylalkynyl, alkoxyalkyl, alkenyl, alkynyl, or alkyl, cycloalkyl, cycloalkenyl, cycloalkylalkyl, cycloalkylalkenyl, cycloalkylalkynyl, cycloalkenylalkyl, cycloalkenylalkenyl, cycloalkenylalkynyl, carboxyalkyl, carboxyalkenyl, carboxyalkynyl, heterocyclyl, heterocyclylalkyl, heterocyclylalkenyl, heterocyclylalkynyl, alkoxyalkyl, alkenyl or alkynyl substituted with one or more halo, aryl, aralkyl, aralkenyl, aralkynyl, or aryl, aralkyl, aralkenyl, aralkynyl substituted with one or more substituents independently selected from halo, alkyl, alkenyl, alkynyl, haloalkyl, haloalkenyl, haloalkynyl, alkoxy and haloalkoxy, heteroaryl, heteroaralkyl, heteroaralkenyl, heteroaralkynyl, or heteroaryl, heteroaralkyl, heteroaralkenyl, heteroaralkynyl substituted with one or more substituents independently selected from halo, alkyl, alkenyl, alkynyl, haloalkyl, haloalkenyl, haloalkynyl, alkoxy and haloalkoxy, or R³ and R⁴ taken together with the nitrogen atom to which they are attached form a 5-or 6-membered saturated or unsaturated heterocyclic ring, and
Y¹ and Y² are each independently halo or OCCl₃.

2. The process of claim 1 wherin Z³ is alkyl, aralkyl or aralkyl substituted with one or more substituents independently selected from halo, alkyl, alkenyl, alkynyl, haloalkyl, haloalkenyl, haloalkynyl, alkoxy and haloalkoxy, and R² is a hydrogen atom.

3. The process of claim 2 wherein Z³ is alkyl or aralkyl.

4. The process of claim 1 wherein
Z³ is (C₁-C₂₀)alkyl, (C₁-C₁₀)alkylcarbonyloxy(C₁-C₁₀)alkyl, hydroxy (C₁-C₂₀)alkyl, (C₁-C₁₀)alkylsulfonyl(C₁-C₁₀)alkyl, acetylamino(C₁-C₁₀) alkyl, halo (C₁-C₂₀)alkyl, (C₂-C₁₀)alkenyl, acetylamino(C₂-C₁₀)alkenyl, halo(C₂-C₁₀)alkenyl, (C₂-C₁₀) alkynyl, halo(C₂-C₁₀)alkynyl, cyclo(C₃-C₈)alkyl, cyclo(C₃-C₈)alkenyl, carboxycyclo(C₃-C₈)alkyl, carboxycyclo(C₃-C₈)alkenyl, cyclo(C₃-C₈)alkyl (C₁-C₁₀)alkyl, cyclo(C₃-C₈)alkyl(C₂-C₁₀)alkenyl, cyclo(C₃-C₈)alkenyl(C₁-C₁₀)alkyl, cyclo(C₃-C₈)alkenyl(C₂-C₁₀)alkenyl, cyclo(C₃-C₈)alkyl(C₂-C₁₀)alkynyl, cyclo (C₃-C₈)alkenyl(C₂-C₁₀)alkynyl, carboxycyclo(C₃-C₈)alkyl(C₁-C₁₀)alkyl, carboxycyclo(C₃-C₈)alkyl(C₂-C₁₀)alkenyl, carboxycyclo(C₃-C₈)alkenyl(C₁-C₁₀)alkyl, carboxycyclo(C₃-C₈)alkenyl(C₂-C₁₀)alkenyl, carboxycyclo(C₃-C₈)alkyl (C₂-C₁₀)alkynyl, carboxycyclo(C₃-C₈)alkenyl(C₂-C₁₀)alkynyl, heterocyclyl, heterocyclyl(C₁-C₁₀)alkyl, heterocyclyl(C₂-C₁₀)alkenyl, heterocyclyl(C₂-C₁₀) alkynyl, (C₁-C₁₀)alkoxy(C₁-C₁₀)alkyl, (C₁-C₁₀)alkoxy(C₁-C₁₀)alkoxy(C₁-C₁₀) alkyl, (C₁-C₁₀)alkoxy(C₂-C₁₀)alkenyl, (C₁-C₁₀)alkoxy(C₂-C₁₀)alkynyl, (C₁-C₁₀) alkoxycarbonyl(C₁-C₁₀)alkyl, (C₁-C₁₀)alkoxycarbonyl(C₂-C₁₀)alkenyl, (C₁-C₁₀) alkoxycarbonyl(C₂-C₁₀)alkynyl, halo(C₁-C₁₀)alkoxy(C₁-C₁₀)alkyl, halo(C₁-C₁₀) alkoxy(C₂-C₁₀)alkenyl, halo(C₁-C₁₀)alkoxy(C₂-C₁₀)alkynyl, (C₁-C₁₀) alkylthio(C₁-C₁₀)alkyl, (C₁-C₁₀)alkylthio(C₂-C₁₀)alkenyl, (C₁-C₁₀)alkylthio(C₂-C₁₀) alkynyl, halo(C₁-C₁₀)alkylthio(C₁-C₁₀)alkyl, halo(C₁-C₁₀)alkylthio(C₂-C₁₀) alkenyl, halo(C₁-C₁₀)alkylthio(C₂-C₁₀)alkynyl, carboxy(C₁-C₂₀)alkyl, carboxy(C₂-C₁₀)alkenyl, carboxy(C₂-C₁₀)alkynyl, SiR³R⁴R⁵, aryl, aryl substituted with one or more substituents independently selected from halo, nitro, hydroxy, cyano, thiocyanato, (C₁-C₁₀)alkyl, (C₁-C₁₀)alkylsulfonyl(C₁-C₁₀)alkyl, (C₂-C₁₀) alkenyl, (C₂-C₁₀)alkynyl, halo(C₁-C₁₀)alkyl, halo(C₂-C₁₀)alkenyl, halo(C₂-C₁₀) alkynyl, (C₁-C₁₀)alkoxy, halo(C₁-C₁₀)alkoxy, SO₂NR³R⁴ and NR³R⁴, ar(C₁-C₁₀) alkyl, ar(C₂-C₁₀)alkenyl, ar(C₂-C₁₀)alkynyl, arcyclo(C₃-C₈)alkyl, aroxy(C₁-C₁₀) alkyl, or ar(C₁-C₁₀)alkyl, ar(C₂-C₁₀)alkenyl, ar(C₂-C₁₀)alkynyl, arcyclo(C₃-C₈) alkyl, aroxy(C₁-C₁₀)alkyl substituted with one or more substituents independently selected from halo, nitro, hydroxy, cyano, (C₁-C₁₀)alkyl, cycloalkyl, (C₂-C₁₀)alkenyl, (C₂-C₁₀)alkynyl, halo(C₁-C₁₀)alkyl, halo(C₂-C₁₀)alkenyl, halo (C₂-C₁₀)alkynyl, (C₁-C₁₀)alkoxy, halo(C₁-C₁₀)alkoxy, SO₂NR³R⁴ and NR³R⁴, heteroaryl, heteroaryl substituted with one or more substituents independently selected from halo, nitro, hydroxy, cyano, (C₁-C₁₀)alkyl, (C₂-C₁₀)alkenyl, (C₂-C₁₀) alkynyl, halo(C₁-C₁₀)alkyl, halo(C₂-C₁₀)alkenyl, halo(C₂-C₁₀)alkynyl, (C₁-C₁₀) alkoxy, halo(C₁-C₁₀)alkoxy, SO₂NR³R⁴ and NR³R⁴, heteroar(C₁-C₁₀)alkyl, heteroar(C₂-C₁₀)alkenyl, heteroar(C₂-C₁₀)alkynyl, or heteroar(C₁-C₁₀)alkyl, heteroar(C₂-C₁₀)alkenyl, heteroar(C₂-C₁₀)alkynyl substituted with one or more substituents independently selected from halo, hydroxy, cyano, nitro, (C₁-C₁₀) alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴,
R² is a hydrogen atom, (C₁-C₂₀)alkyl, (C₂-C₁₀)alkenyl, (C₂-C₁₀)alkynyl, (C₁-C₁₀) alkoxy(C₁-C₁₀)alkyl, (C₁-C₁₀)alkoxy(C₂-C₁₀)alkenyl, (C₁-C₁₀)alkoxy(C₂-C₁₀) alkynyl, (C₁-C₁₀)alkylthio(C₁-C₁₀)alkyl, (C₁-C₁₀)alkylthio(C₂-C₁₀)alkenyl, (C₁-C₁₀) alkylthio(C₂-C₁₀)alkynyl, carboxy, a carboxylate salt, carboxy(C₁-C₂₀)alkyl, carboxy(C₂-C₂₀)alkenyl, carboxy(C₂-C₂₀)alkynyl, (C₁-C₂₀)alkoxycarbonyl, (C₁-C₁₀) alkoxycarbonyl(C₁-C₁₀)alkyl, (C₁-C₁₀)alkoxycarbonyl(C₂-C₁₀)alkenyl, (C₁-C₁₀) alkoxycarbonyl(C₂-C₁₀)alkynyl, (C₁-C₂₀)alkylcarbonyl, (C₂-C₂₀) alkenylcarbonyl, (C₂-C₂₀)alkynylcarbonyl, cyclo(C₃-C₈)alkyl, cyclo(C₃-C₈) alkenyl, cyclo(C₃-C₈)alkyl(C₁-C₁₀)alkyl, cyclo(C₃-C₈)alkenyl(C₁-C₁₀)alkyl, cyclo(C₃-C₈)alkyl(C₂-C₁₀)alkenyl, cyclo(C₃-C₈)alkenyl(C₂-C₁₀)alkenyl, cyclo(C₃-C₈) alkyl(C₂-C₁₀)alkynyl, cyclo(C₃-C₈)alkenyl(C₂-C₁₀)alkynyl, heterocyclyl, heterocyclyl(C₁-C₁₀)alkyl, heterocyclyl(C₂-C₁₀)alkenyl, heterocyclyl(C₂-C₁₀) alkynyl, or (C₁-C₂₀)alkyl, (C₂-C₁₀)alkenyl, (C₂-C₁₀)alkynyl, (C₁-C₁₀)alkoxy(C₁-C₁₀) alkyl, (C₁-C₁₀)alkoxy(C₂-C₁₀)alkenyl, (C₁-C₁₀)alkoxy(C₂-C₁₀)alkynyl, (C₁-C₁₀) alkylthio(C₁-C₁₀)alkyl, (C₁-C₁₀)alkylthio(C₂-C₁₀)alkenyl, (C₁-C₁₀)alkylthio (C₂-C₁₀)alkynyl, carboxy(C₁-C₂₀)alkyl, carboxy(C₂-C₁₀)alkenyl, carboxy(C₂-C₁₀) alkynyl, (C₁-C₂₀)alkoxycarbonyl, (C₁-C₁₀)alkoxycarbonyl(C₁-C₁₀)alkyl, (C₁-C₁₀) alkoxycarbonyl(C₂-C₁₀)alkenyl, (C₁-C₁₀)alkoxycarbonyl(C₂-C₁₀)alkynyl, (C₁-C₂₀) alkylcarbonyl, (C₂-C₁₀)alkenylcarbonyl, (C₂-C₁₀)alkynylcarbonyl, cyclo(C₃-C₈) alkyl, cyclo(C₃-C₈)alkenyl, cyclo(C₃-C₈)alkyl(C₁-C₁₀)alkyl, cyclo(C₃-C₈) alkenyl(C₁-C₁₀)alkyl, cyclo(C₃-C₈)alkyl(C₂-C₁₀)alkenyl, cyclo(C₃-C₈)alkenyl (C₂-C₁₀)alkenyl, cyclo(C₃-C₈)alkyl(C₂-C₁₀)alkynyl, cyclo(C₃-C₈)alkenyl(C₂-C₁₀)alkynyl, heterocyclyl, heterocyclyl(C₁-C₁₀)alkyl, heterocyclyl(C₂-C₁₀)alkenyl, heterocyclyl(C₂-C₁₀)alkynyl substituted with one or more substituents independently selected from halo, cyano, hydroxy, nitro, SO₂NR³R⁴ and NR³R⁴, aryl or aryl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, carboxy, (C₁-C₄)alkoxycarbonyl, SO₂NR³R⁴ and NR³R⁴, ar(C₁-C₁₀)alkyl, ar (C₂-C₁₀)alkenyl, ar(C₂-C₁₀)alkynyl, or ar(C₁-C₁₀)alkyl, ar(C₂-C₁₀)alkenyl, ar (C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, arylcarbonyl, ar(C₁-C₁₀)alkylcarbonyl, ar(C₂-C₁₀) alkenylcarbonyl, ar(C₂-C₁₀)alkynylcarbonyl or arylcarbonyl, ar(C₁-C₁₀) alkylcarbonyl, ar(C₂-C₁₀)alkenylcarbonyl, ar(C₂-C₁₀)alkynylcarbonyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀) alkyl, halo(C₂-C₁₀)alkenyl, halo(C₂-C₁₀)alkynyl, (C₁-C₁₀)alkoxy, halo(C₁-C₁₀) alkoxy, SO₂NR³R⁴ and NR³R⁴, heteroaryl, heteroaryl substituted with one or more substituents independently selected from halo, (C₁-C₁₀)alkyl, (C₂-C₁₀) alkenyl, (C₂-C₁₀)alkynyl, halo(C₁-C₁₀)alkyl, halo(C₂-C₁₀)alkenyl, halo(C₂-C₁₀) alkynyl, (C₁-C₁₀)alkoxy, halo(C₁-C₁₀)alkoxy and NR³R⁴, heteroar(C₁-C₁₀)alkyl, heteroar(C₂-C₁₀)alkenyl, heteroar(C₂-C₁₀)alkynyl or heteroar(C₁-C₁₀)alkyl, heteroar(C₂-C₁₀)alkenyl, heteroar(C₂-C₁₀)alkynyl substituted with one or more substituents independently selected from halo, (C₁-C₁₀)alkyl, (C₂-C₁₀)alkenyl, (C₂-C₁₀) alkynyl, halo(C₁-C₁₀)alkyl, halo(C₂-C₁₀)alkenyl, halo(C₂-C₁₀)alkynyl, (C₁-C₁₀) alkoxy, halo(C₁-C₁₀)alkoxy, SO₂NR³R⁴ and NR³R⁴, heteroar(C₁-C₁₀) alkylcarbonyl, heteroar(C₂-C₁₀)alkenylcarbonyl, heteroar(C₂-C₁₀) alkynylcarbonyl or heteroar(C₁-C₁₀)alkylcarbonyl, heteroar(C₂-C₁₀) alkenylcarbonyl, heteroar(C₂-C₁₀)alkynylcarbonyl substituted with one or more substituents independently selected from halo, cyano, hydroxy, nitro, (C₁-C₁₀) alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, or R¹ and R² taken together with the carbon atom to which they are attached form a 5-7 membered saturated or unsaturated ring,
R³, R⁴ and R⁵ are each independently a hydrogen atom, (C₁-C₂₀)alkyl, cyclo(C₃-C₈)alkyl, cyclo(C₃-C₈)alkenyl, cyclo(C₃-C₈)alkyl(C₁-C₁₀)alkyl, cyclo(C₃-C₈) alkyl(C₂-C₁₀)alkenyl, cyclo(C₃-C₈)alkyl(C₂-C₁₀)alkynyl, cyclo(C₃-C₈)alkenyl(C₁-C₁₀) alkyl, cyclo(C₃-C₈)alkenyl(C₂-C₁₀)alkenyl, cyclo(C₃-C₈)alkenyl(C₂-C₁₀)alkynyl, carboxy(C₁-C₂₀)alkyl, carboxy(C₂-C₁₀)alkenyl, carboxy(C₂-C₁₀)alkynyl, heterocyclyl, heterocyclyl(C₁-C₁₀)alkyl, heterocyclyl(C₂-C₁₀)alkenyl, heterocyclyl(C₂-C₁₀)alkynyl, (C₁-C₁₀)alkoxy(C₁-C₁₀)alkyl, (C₂-C₁₀)alkenyl, (C₂-C₁₀) alkynyl, or (C₁-C₁₀)alkyl, cyclo(C₃-C₈)alkyl, cyclo(C₃-C₈)alkenyl, cyclo(C₃-C₈) alkyl(C₁-C₁₀)alkyl, cyclo(C₃-C₈)alkyl(C₂-C₁₀)alkenyl, cyclo(C₃-C₈)alkyl(C₂-C₁₀) alkynyl, cyclo(C₃-C₈)alkenyl(C₁-C₁₀)alkyl, cyclo(C₃-C₈)alkenyl(C₂-C₁₀)alkenyl, cyclo(C₃-C₈)alkenyl(C₂-C₁₀)alkynyl, carboxy(C₁-C₂₀)alkyl, carboxy(C₂-C₁₀)alkenyl, carboxy(C₂-C₁₀)alkynyl, heterocyclyl, heterocyclyl(C₁-C₁₀)alkyl, heterocyclyl(C₂-C₁₀) alkenyl, heterocyclyl(C₂-C₁₀)alkynyl, (C₁-C₁₀)alkoxy(C₁-C₁₀)alkyl, (C₂-C₁₀) alkenyl or (C₂-C₁₀)alkynyl substituted with one or more halo, aryl, ar(C₁-C₁₀)alkyl, ar(C₂-C₁₀)alkenyl, ar(C₂-C₁₀)alkynyl or aryl, ar(C₁-C₁₀)alkyl, ar(C₂-C₁₀) alkenyl, ar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy and halo(C₁-C₁₀)alkoxy, heteroaryl, heteroar(C₁-C₁₀)alkyl, heteroar(C₂-C₁₀)alkenyl, heteroar(C₂-C₁₀)alkynyl or heteroaryl, heteroar(C₁-C₁₀)alkyl, heteroar(C₂-C₁₀) alkenyl, heteroar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy and halo(C₁-C₁₀)alkoxy, or R³ and R⁴ taken together with the nitrogen atom to which they are attached form a 5-or 6-membered saturated or unsaturated heterocyclic ring, and
Y¹ and Y² are each independently halo or OCCl₃.

5. The process of claim 4 wherein Z³ is (C₁-C₂₀)alkyl, ar(C₁-C₁₀)alkyl or ar(C₁-C₁₀) alkyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀) alkenyl, halo(C₂-C₁₀)alkynyl, (C₁-C₁₀)alkoxy and halo(C₁-C₁₀)alkoxy, and R² is a hydrogen atom.

6. The process of claim 5 wherein Z³ is (C₁-C₁₀)alkyl or ar(C₁-C₆)alkyl.

7. The process of any one of claims 1-6 wherein the suitable catalyst is a pyridine, a thiourea, a urea, a phosphoramide, a substituted amide, a quaternary ammonium halide, an arylamine, a tertiary phosphine, an alkali metal halide or an alkaline earth metal halide.

8. The process of claim 7 wherein pyridine is the catalyst.

9. The process of claim 7 wherein the suitable solvent is an ether, acetonitrile, a chlorinated solvent or a mixture thereof.

10. The process of claim 7 wherein the reaction temperature is between from about −78° C. to about 100° C.

11. The process of claim 7 wherein from about 1 to about 10 equivalents of a compound of formula (VIII) are used per equivalent of a compound of formula (VII).

12. A process leading to a compound of formula (III) 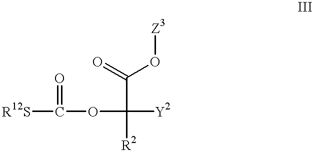
comprising the step of reacting, in the presence of a suitable solvent, a compound of formula (V) 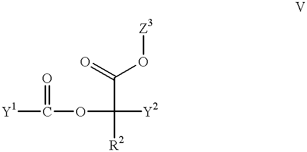
with a compound of formula (VI) 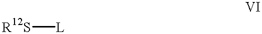
wherein
Z³ is alkyl, alkylcarbonyloxyalkyl, hydroxyalkyl, alkylsulfonylalkyl, acetylaminoalkyl, haloalkyl, alkenyl, acetylaminoalkenyl, haloalkenyl, alkynyl, haloalkynyl, cycloalkyl, cycloalkenyl, carboxycycloalkyl, carboxycycloalkenyl, cycloalkylalkyl, cycloalkylalkenyl, cycloalkenylalkyl, cycloalkenylalkenyl, cycloalkylalkynyl, cycloalkenylalkynyl, carboxycycloalkylalkyl, carboxycycloalkylalkenyl, carboxycycloalkenylalkyl, carboxycycloalkenylalkenyl, carboxycycloalkylalkynyl, carboxycycloalkenylalkynyl, heterocyclyl, heterocyclylalkyl, heterocyclylalkenyl, heterocyclylalkynyl, alkoxyalkyl, alkoxyalkoxyalkyl, alkoxyalkenyl, alkoxyalkynyl, alkoxycarbonylalkyl, alkoxycarbonylalkenyl, alkoxycarbonylalkynyl, haloalkoxyalkyl, haloalkoxyalkenyl, haloalkoxyalkynyl, alkylthioalkyl, alkylthioalkenyl, alkylthioalkynyl, haloalkylthioalkyl, haloalkylthioalkenyl, haloalkylthioalkynyl, carboxyalkyl, carboxyalkenyl, carboxyalkynyl, SiR³R⁴R⁵, aryl, aryl substituted with one or more substituents independently selected from halo, nitro, hydroxy, cyano, thiocyanato, alkyl, alkylsulfonylalkyl, alkenyl, alkynyl, haloalkyl, haloalkenyl, haloalkynyl, alkoxy, haloalkoxy, SO₂NR³R⁴ and NR³R⁴, aralkyl, aralkenyl, aralkynyl, arcycloalkyl, aroxyalkyl, or aralkyl, aralkenyl, aralkynyl, arcycloalkyl, aroxyalkyl substituted with one or more substituents independently selected from halo, nitro, hydroxy, cyano, alkyl, cycloalkyl, alkenyl, alkynyl, haloalkyl, haloalkenyl, haloalkynyl, alkoxy, haloalkoxy, SO₂NR³R⁴ and NR³R⁴, heteroaryl, heteroaryl substituted with one or more substituents independently selected from halo, nitro, hydroxy, cyano, alkyl, alkenyl, alkynyl, haloalkyl, haloalkenyl, haloalkynyl, alkoxy, haloalkoxy, SO₂NR³R⁴ and NR³R⁴, heteroaralkyl, heteroaralkenyl, heteroaralkynyl, or heteroaralkyl, heteroaralkenyl, heteroaralkynyl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴,
R² is a hydrogen atom, alkyl, alkenyl, alkynyl, alkoxyalkyl, alkoxyalkenyl, alkoxyalkynyl, alkylthioalkyl, alkylthioalkenyl, alkylthioalkynyl, carboxy, a carboxylate salt, carboxyalkyl, carboxyalkenyl, carboxyalkynyl, alkoxycarbonyl, alkoxycarbonylalkyl, alkoxycarbonylalkenyl, alkoxycarbonylalkynyl, alkylcarbonyl, alkenylcarbonyl, alkynylcarbonyl, cycloalkyl, cycloalkenyl, cycloalkylalkyl, cycloalkenylalkyl, cycloalkylalkenyl, cycloalkenylalkenyl, cycloalkylalkynyl, cycloalkenylalkynyl, heterocyclyl, heterocyclylalkyl, heterocyclylalkenyl, heterocyclylalkynyl, or alkyl, alkenyl, alkynyl, alkoxyalkyl, alkoxyalkenyl, alkoxyalkynyl, alkylthioalkyl, alkylthioalkenyl, alkylthioalkynyl, carboxyalkyl, carboxyalkenyl, carboxyalkynyl, alkoxycarbonyl, alkoxycarbonylalkyl, alkoxycarbonylalkenyl, alkoxycarbonylalkynyl, alkylcarbonyl, alkenylcarbonyl, alkynylcarbonyl, cycloalkyl, cycloalkenyl, cycloalkylalkyl, cycloalkenylalkyl, cycloalkylalkenyl, cycloalkenylalkenyl, cycloalkylalkynyl, cycloalkenylalkynyl, heterocyclyl, heterocyclylalkyl, heterocyclylalkenyl, heterocyclylalkynyl substituted with one or more substituents independently selected from halo, cyano, hydroxy, nitro, SO₂NR³R⁴ and NR³R⁴, aryl or aryl substituted with one or more substituents independently selected from halo, hydroxy, cyano, nitro, alkyl, alkenyl, alkynyl, haloalkyl, haloalkenyl, haloalkynyl, alkoxy, haloalkoxy, carboxy, alkoxycarbonyl, SO₂NR³R⁴ and NR³R⁴, aralkyl, aralkenyl, aralkynyl or aralkyl, aralkenyl, aralkynyl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, arylcarbonyl, aralkylcarbonyl, aralkenylcarbonyl, aralkynylcarbonyl or arylcarbonyl, aralkylcarbonyl, aralkenylcarbonyl, aralkynylcarbonyl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, heteroaryl or heteroaryl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, heteroaralkyl, heteroaralkenyl, heteroaralkynyl or heteroaralkyl, heteroaralkenyl, heteroaralkynyl substituted with one or more substituents independently selected from halo, cyano, hydroxy, nitro, alkyl, alkenyl, alkynyl, haloalkyl, haloalkenyl, haloalkynyl, alkoxy, haloalkoxy, SO₂NR³R⁴ and NR³R⁴, heteroaralkylcarbonyl, heteroaralkenylcarbonyl, heteroaralkynylcarbonyl or heteroaralkylcarbonyl, heteroaralkenylcarbonyl, heteroaralkynylcarbonyl substituted with one or more substituents independently selected from halo, cyano, hydroxy, nitro, alkyl, alkenyl, alkynyl, haloalkyl, haloalkenyl, haloalkynyl, alkoxy, haloalkoxy, SO₂NR³R⁴ and NR³R⁴,
R³, R⁴ and R⁵ are each independently a hydrogen atom, alkyl, cycloalkyl, cycloalkenyl, cycloalkylalkyl, cycloalkylalkenyl, cycloalkylalkynyl, cycloalkenylalkyl, cycloalkenylalkenyl, cycloalkenylalkynyl, carboxyalkyl, carboxyalkenyl, carboxyalkynyl, heterocyclyl, heterocyclylalkyl, heterocyclylalkenyl, heterocyclylalkynyl, alkoxyalkyl, alkenyl, alkynyl, or alkyl, cycloalkyl, cycloalkenyl, cycloalkylalkyl, cycloalkylalkenyl, cycloalkylalkynyl, cycloalkenylalkyl, cycloalkenylalkenyl, cycloalkenylalkynyl, carboxyalkyl, carboxyalkenyl, carboxyalkynyl, heterocyclyl, heterocyclylalkyl, heterocyclylalkenyl, heterocyclylalkynyl, alkoxyalkyl, alkenyl or alkynyl substituted with one or more halo, aryl, aralkyl, aralkenyl, aralkynyl, or aryl, aralkyl, aralkenyl, aralkynyl substituted with one or more substituents independently selected from halo, alkyl, alkenyl, alkynyl, haloalkyl, haloalkenyl, haloalkynyl, alkoxy and haloalkoxy, heteroaryl, heteroaralkyl, heteroaralkenyl, heteroaralkynyl, or heteroaryl, heteroaralkyl, heteroaralkenyl, heteroaralkynyl substituted with one or more substituents independently selected from halo, alkyl, alkenyl, alkynyl, haloalkyl, haloalkenyl, haloalkynyl, alkoxy and haloalkoxy, or R³ and R⁴ taken together with the nitrogen atom to which they are attached form a 5-or 6-membered saturated or unsaturated heterocyclic ring,
R¹² is alkyl, cycloalkyl, cycloalkenyl, cycloalkylalkyl, cycloalkylalkenyl, cycloalkylalkynyl, cycloalkenylalkyl, cycloalkenylalkenyl, cycloalkenylalkynyl, carboxyalkyl, carboxyalkenyl, carboxyalkynyl, heterocyclyl, heterocyclylalkyl, heterocyclylalkenyl, heterocyclylalkynyl, alkoxyalkyl, alkenyl, alkynyl, or alkyl, cycloalkyl, cycloalkenyl, cycloalkylalkyl, cycloalkylalkenyl, cycloalkylalkynyl, cycloalkenylalkyl, cycloalkenylalkenyl, cycloalkenylalkynyl, carboxyalkyl, carboxyalkenyl, carboxyalkynyl, heterocyclyl, heterocyclylalkyl, heterocyclylalkenyl, heterocyclylalkynyl, alkoxyalkyl, alkenyl or alkynyl substituted with one or more halo, aryl or aryl substituted with one or more substituents independently selected from halo, nitro, cyano, haloalkyl, haloalkenyl, haloalkynyl, alkylcarbonyl, alkenylcarbonyl, alkynylcarbonyl, arylcarbonyl, alkylsulfonyl and arylsulfonyl, aralkyl, aralkenyl, aralkynyl, or aralkyl, aralkenyl, aralkynyl substituted with one or more substituents independently selected from halo, alkyl, alkenyl, alkynyl, haloalkyl, haloalkenyl, haloalkynyl, alkoxy and haloalkoxy, heteroaryl, heteroaralkyl, heteroaralkenyl, heteroaralkynyl, or heteroaryl, heteroaralkyl, heteroaralkenyl, heteroaralkynyl substituted with one or more substituents independently selected from halo, alkyl, alkenyl, alkynyl, haloalkyl, haloalkenyl, haloalkynyl, alkoxy and haloalkoxy,
Y¹ and Y² are each independently halo, and
L is a hydrogen atom, a sodium atom or a potassium atom.

13. The process of claim 12 wherein
Y¹ is chloro,
Y² is chloro, bromo or iodo,
L is a sodium atom or a potassium atom,
R¹² is alkyl, aralkyl, aryl or heteroaryl, or aralkyl, aryl or heteroaryl substituted with one or more substituents independently selected from halo, alkyl, alkenyl, alkynyl, haloalkyl, haloalkenyl, haloalkynyl, alkoxy and haloalkoxy,
Z³ is alkyl, aryl, aralkyl or aryl or aralkyl substituted with one or more substituents independently selected from halo, alkyl, alkenyl, alkynyl, haloalkyl, haloalkenyl, haloalkynyl, alkoxy and haloalkoxy, and
R² is a hydrogen atom.

14. The process of claim 13 wherein R¹² is alkyl, L is sodium and Z³ is alkyl or aralkyl.

15. The process of claim 12 wherein
Z³ is (C₁-C₂₀)alkyl, (C₁-C₁₀)alkylcarbonyloxy(C₁-C₁₀)alkyl, hydroxy(C₁-C₂₀) alkyl, (C₁-C₁₀)alkylsulfonyl(C₁-C₁₀)alkyl, acetylamino(C₁-C₁₀)alkyl, halo(C₁-C₂₀) alkyl, (C₂-C₁₀)alkenyl, acetylamino(C₂-C₁₀)alkenyl, halo(C₂-C₁₀)alkenyl, (C₂-C₁₀) alkynyl, halo(C₂-C₁₀)alkynyl, cyclo(C₃-C₈)alkyl, cyclo(C₃-C₈)alkenyl, carboxycyclo(C₃-C₈)alkyl, carboxycyclo(C₃-C₈)alkenyl, cyclo(C₃-C₈)alkyl(C₁-C₁₀)alkyl, cyclo(C₃-C₈)alkyl(C₂-C₁₀)alkenyl, cyclo(C₃-C₈)alkenyl(C₁-C₁₀)alkyl, cyclo(C₃-C₈)alkenyl(C₂-C₁₀)alkenyl, cyclo(C₃-C₈)alkyl(C₂-C₁₀)alkynyl, cyclo(C₃-C₈) alkenyl(C₂-C₁₀)alkynyl, carboxycyclo(C₃-C₈)alkyl(C₁-C₁₀)alkyl, carboxycyclo(C₃-C₈)alkyl(C₂-C₁₀)alkenyl, carboxycyclo(C₃-C₈)alkenyl(C₁-C₁₀)alkyl, carboxycyclo(C₃-C₈)alkenyl(C₂-C₁₀)alkenyl, carboxycyclo(C₃-C₈)alkyl(C₂-C₁₀) alkynyl, carboxycyclo(C₃-C₈)alkenyl(C₂-C₁₀)alkynyl, heterocyclyl, heterocyclyl(C₁C₁₀)alkyl, heterocyclyl(C₂-C₁₀)alkenyl, heterocyclyl(C₂-C₁₀) alkynyl, (C₁-C₁₀)alkoxy(C₁-C₁₀)alkyl, (C₁-C₁₀)alkoxy(C₁-C₁₀)alkoxy(C₁-C₁₀) alkyl, (C₁-C₁₀)alkoxy(C₂-C₁₀)alkenyl, (C₁-C₁₀)alkoxy(C₂-C₁₀)alkynyl, (C₁-C₁₀) alkoxycarbonyl(C₁-C₁₀)alkyl, (C₁-C₁₀)alkoxycarbonyl(C₂-C₁₀)alkenyl, (C₁-C₁₀) alkoxycarbonyl(C₂-C₁₀)alkynyl, halo(C₁-C₁₀)alkoxy(C₁-C₁₀)alkyl, halo(C₁-C₁₀) alkoxy(C₂-C₁₀)alkenyl, halo(C₁-C₁₀)alkoxy(C₂-C₁₀)alkynyl, (C₁-C₁₀) alkylthio(C₁-C₁₀)alkyl, (C₁-C₁₀)alkylthio(C₂-C₁₀)alkenyl, (C₁-C₁₀)alkylthio(C₂-C₁₀) alkynyl, halo(C₁-C₁₀)alkylthio(C₁-C₁₀)alkyl, halo(C₁-C₁₀)alkylthio(C₂-C₁₀) alkenyl, halo(C₁-C₁₀)alkylthio(C₂-C₁₀)alkynyl, carboxy(C₁-C₂₀)alkyl, carboxy(C₂-C₁₀)alkenyl, carboxy(C₂-C₁₀)alkynyl, SiR³R⁴R⁵, aryl, aryl substituted with one or more substituents independently selected from halo, nitro, hydroxy, cyano, thiocyanato, (C₁-C₁₀)alkyl, (C₁-C₁₀)alkylsulfonyl(C₁-C₁₀)alkyl, (C₂-C₁₀) alkenyl, (C₂-C₁₀)alkynyl, halo(C₁-C₁₀)alkyl, halo(C₂-C₁₀)alkenyl, halo(C₂-C₁₀) alkynyl, (C₁-C₁₀)alkoxy, halo(C₁-C₁₀)alkoxy, SO₂NR³R⁴ and NR³R⁴, ar(C₁-C₁₀) alkyl, ar(C₂-C₁₀)alkenyl, ar(C₂-C₁₀)alkynyl, arcyclo(C₃-C₈)alkyl, aroxy(C₁-C₁₀) alkyl, or ar(C₁-C₁₀)alkyl, ar(C₂-C₁₀)alkenyl, ar(C₂-C₁₀)alkynyl, arcyclo(C₃-C₈) alkyl, aroxy(C₁-C₁₀)alkyl substituted with one or more substituents independently selected from halo, nitro, hydroxy, cyano, (C₁-C₁₀)alkyl, cycloalkyl, (C₂-C₁₀)alkenyl, (C₂-C₁₀)alkynyl, halo(C₁C₁₀)alkyl, halo(C₂-C₁₀)alkenyl, halo(C₂-C₁₀) alkynyl, (C₁-C₁₀)alkoxy, halo(C₁-C₁₀)alkoxy, SO₂NR³R⁴ and NR³R⁴, heteroaryl, heteroaryl substituted with one or more substituents independently selected from halo, nitro, hydroxy, cyano, (C₁-C₁₀)alkyl, (C₂-C₁₀)alkenyl, (C₂-C₁₀) alkynyl, halo(C₁-C₁₀)alkyl, halo(C₂-C₁₀)alkenyl, halo(C₂-C₁₀)alkynyl, (C₁-C₁₀) alkoxy, halo(C₁-C₁₀)alkoxy, SO₂NR³R⁴ and NR³R⁴, heteroar(C₁-C₁₀)alkyl, heteroar(C₂-C₁₀)alkenyl, heteroar(C₂-C₁₀)alkynyl, or heteroar(C₁-C₁₀)alkyl, heteroar(C₂-C₁₀)alkenyl, heteroar(C₂-C₁₀)alkynyl substituted with one or more substituents independently selected from halo, hydroxy, cyano, nitro, (C₁-C₁₀) alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴,
R² is a hydrogen atom, (C₁-C₂₀)alkyl, (C₂-C₁₀)alkenyl, (C₂-C₁₀)alkynyl, (C₁-C₁₀) alkoxy(C₁-C₁₀)alkyl, (C₁-C₁₀)alkoxy(C₂-C₁₀)alkenyl, (C₁-C₁₀)alkoxy(C₂-C₁₀) alkynyl, (C₁-C₁₀)alkylthio(C₁-C₁₀)alkyl, (C₁-C₁₀)alkylthio(C₂-C₁₀)alkenyl, (C₁-C₁₀) alkylthio(C₂-C₁₀)alkynyl, carboxy, a carboxylate salt, carboxy(C₁-C₂₀)alkyl, carboxy(C₂-C₂₀)alkenyl, carboxy(C₂-C₂₀)alkynyl, (C₁-C₂₀)alkoxycarbonyl, (C₁-C₁₀) alkoxycarbonyl(C₁-C₁₀alkyl, (C₁-C₁₀)alkoxycarbonyl(C₂-C₁₀)alkenyl, (C₁-C₁₀) alkoxycarbonyl(C₂-C₁₀)alkynyl, (C₁-C₂₀)alkylcarbonyl, (C₂-C₂₀) alkenylcarbonyl, (C₂-C₂₀)alkynylcarbonyl, cyclo(C₃-C₈)alkyl, cyclo(C₃-C₈) alkenyl, cyclo(C₃-C₈)alkyl(C₁-C₁₀)alkyl, cyclo(C₃-C₈)alkenyl(C₁-C₁₀)alkyl, cyclo(C₃-C₈)alkyl(C₂-C₁₀)alkenyl, cyclo(C₃-C₈)alkenyl(C₂-C₁₀)alkenyl, cyclo(C₃-C₈) alkyl(C₂-C₁₀)alkynyl, cyclo(C₃-C₈)alkenyl(C₂-C₁₀)alkynyl, heterocyclyl, heterocyclyl(C₁-C₁₀)alkyl, heterocyclyl(C₂-C₁₀)alkenyl, heterocyclyl(C₂-C₁₀) alkynyl, or (C₁-C₂₀)alkyl, (C₂-C₁₀)alkenyl, (C₂-C₁₀)alkynyl, (C₁-C₁₀)alkoxy(C₁-C₁₀) alkyl, (C₁-C₁₀)alkoxy(C₂-C₁₀)alkenyl, (C₁-C₁₀)alkoxy(C₂-C₁₀)alkynyl, (C₁-C₁₀) alkylthio(C₁-C₁₀)alkyl, (C₁-C₁₀)alkylthio(C₂-C₁₀)alkenyl, (C₁-C₁₀)alkylthio(C₂-C₁₀) alkynyl, carboxy(C₁-C₂₀)alkyl, carboxy(C₂-C₁₀)alkenyl, carboxy(C₂-C₁₀) alkynyl, (C₁-C₂₀)alkoxycarbonyl, (C₁-C₁₀)alkoxycarbonyl(C₁-C₁₀)alkyl, (C₁-C₁₀) alkoxycarbonyl(C₂-C₁₀)alkenyl, (C₁-C₁₀)alkoxycarbonyl(C₂-C₁₀)alkynyl, (C₁-C₂₀) alkylcarbonyl, (C₂-C₁₀)alkenylcarbonyl, (C₂-C₁₀)alkynylcarbonyl, cyclo(C₃-C₈) alkyl, cyclo(C₃-C₈)alkenyl, cyclo(C₃-C₈)alkyl(C₁-C₁₀)alkyl, cyclo(C₃-C₈) alkenyl(C₁-C₁₀)alkyl, cyclo(C₃-C₈)alkyl(C₂-C₁₀)alkenyl, cyclo(C₃-C₈)alkenyl(C₂-C₁₀) alkenyl, cyclo(C₃-C₈)alkyl(C₂-C₁₀)alkynyl, cyclo(C₃-C₈)alkenyl(C₂-C₁₀)alkynyl, heterocyclyl, heterocyclyl(C₁-C₁₀)alkyl, heterocyclyl(C₂-C₁₀)alkenyl, heterocyclyl(C₂-C₁₀)alkynyl substituted with one or more substituents independently selected from halo, cyano, hydroxy, nitro, SO₂NR³R⁴ and NR³R⁴, aryl or aryl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, carboxy, (C₁-C₄)alkoxycarbonyl, SO₂NR³R⁴ and NR³R⁴, ar(C₁-C₁₀)alkyl, ar(C₂-C₁₀) alkenyl, ar(C₂-C₁₀)alkynyl, or ar(C₁-C₁₀)alkyl, ar(C₂-C₁₀)alkenyl, ar(C₂-C₁₀) alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, arylcarbonyl, ar(C₁-C₁₀)alkylcarbonyl, ar(C₂-C₁₀) alkenylcarbonyl, ar(C₂-C₁₀)alkynylcarbonyl or arylcarbonyl, ar(C₁-C₁₀) alkylcarbonyl, ar(C₂-C₁₀)alkenylcarbonyl, ar(C₂-C₁₀)alkynylcarbonyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀) alkyl, halo(C₂-C₁₀)alkenyl, halo(C₂-C₁₀)alkynyl, (C₁-C₁₀)alkoxy, halo(C₁-C₁₀) alkoxy, SO₂NR³R⁴ and NR³R⁴, heteroaryl, heteroaryl substituted with one or more substituents independently selected from halo, (C₁-C₁₀)alkyl, (C₂-C₁₀) alkenyl, (C₂-C₁₀)alkynyl, halo(C₁-C₁₀)alkyl, halo(C₂-C₁₀)alkenyl, halo(C₂-C₁₀) alkynyl, (C₁-C₁₀)alkoxy, halo(C₁-C₁₀)alkoxy and NR³R⁴, heteroar(C₁-C₁₀)alkyl, heteroar(C₂-C₁₀)alkenyl, heteroar(C₂-C₁₀)alkynyl or heteroar(C₁-C₁₀)alkyl, heteroar(C₂-C₁₀)alkenyl, heteroar(C₂-C₁₀)alkynyl substituted with one or more substituents independently selected from halo, (C₁-C₁₀)alkyl, (C₂-C₁₀)alkenyl, (C₂-C₁₀) alkynyl, halo(C₁-C₁₀)alkyl, halo(C₂-C₁₀)alkenyl, halo(C₂-C₁₀)alkynyl, (C₁-C₁₀) alkoxy, halo(C₁-C₁₀)alkoxy, SO₂NR³R⁴ and NR³R⁴, heteroar(C₁-C₁₀) alkylcarbonyl, heteroar(C₂-C₁₀)alkenylcarbonyl, heteroar(C₂-C₁₀) alkynylcarbonyl or heteroar(C₁-C₁₀)alkylcarbonyl, heteroar(C₂-C₁₀) alkenylcarbonyl, heteroar(C₂-C₁₀)alkynylcarbonyl substituted with one or more substituents independently selected from halo, cyano, hydroxy, nitro, (C₁-C₁₀) alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, or R¹ and R² taken together with the carbon atom to which they are attached form a 5-7 membered saturated or unsaturated ring,
R³, R⁴ and R⁵ are each independently a hydrogen atom, (C₁-C₂₀)alkyl, cyclo(C₃-C₈)alkyl, cyclo(C₃-C₈)alkenyl, cyclo(C₃-C₈)alkyl(C₁-C₁₀)alkyl, cyclo(C₃-C₈) alkyl(C₂-C₁₀)alkenyl, cyclo(C₃-C₈)alkyl(C₂-C₁₀)alkynyl, cyclo(C₃-C₈)alkenyl(C₁-C₁₀) alkyl, cyclo(C₃-C₈)alkenyl(C₂-C₁₀)alkenyl, cyclo(C₃-C₈)alkenyl(C₂-C₁₀)alkynyl, carboxy(C₁-C₂₀)alkyl, carboxy(C₂-C₁₀)alkenyl, carboxy(C₂-C₁₀)alkynyl, heterocyclyl, heterocyclyl(C₁-C₁₀)alkyl, heterocyclyl(C₂-C₁₀)alkenyl, heterocyclyl(C₂-C₁₀)alkynyl, (C₁-C₁₀)alkoxy(C₁-C₁₀)alkyl, (C₂-C₁₀)alkenyl, (C₂-C₁₀) alkynyl, or (C₁-C₁₀)alkyl, cyclo(C₃-C₈)alkyl, cyclo(C₃-C₈)alkenyl, cyclo(C₃-C₈) alkyl(C₁-C₁₀)alkyl, cyclo(C₃-C₈)alkyl(C₂-C₁₀)alkenyl, cyclo(C₃-C₈)alkyl(C₂-C₁₀) alkynyl, cyclo(C₃-C₈)alkenyl(C₁-C₁₀)alkyl, cyclo(C₃-C₈)alkenyl(C₂-C₁₀)alkenyl, cyclo(C₃-C₈)alkenyl(C₂-C₁₀)alkynyl, carboxy(C₁-C₂₀)alkyl, carboxy(C₂-C₁₀)alkenyl, carboxy(C₂-C₁₀)alkynyl, heterocyclyl, heterocyclyl(C₁-C₁₀)alkyl, heterocyclyl(C₂-C₁₀) alkenyl, heterocyclyl(C₂-C₁₀)alkynyl, (C₁-C₁₀)alkoxy(C₁-C₁₀)alkyl, (C₂-C₁₀) alkenyl or (C₂-C₁₀)alkynyl substituted with one or more halo, aryl, ar(C₁-C₁₀) alkyl, ar(C₂-C₁₀)alkenyl, ar(C₂-C₁₀)alkynyl or aryl, ar(C₁-C₁₀)alkyl, ar(C₂-C₁₀) alkenyl, ar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy and halo(C₁-C₁₀)alkoxy, heteroaryl, heteroar(C₁-C₁₀)alkyl, heteroar(C₂-C₁₀)alkenyl, heteroar(C₂-C₁₀)alkynyl or heteroaryl, heteroar(C₁-C₁₀)alkyl, heteroar(C₂-C₁₀) alkenyl, heteroar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy and halo(C₁-C₁₀)alkoxy, or R³ and R⁴ taken together with the nitrogen atom to which they are attached form a 5-or 6-membered saturated or unsaturated heterocyclic ring,
R¹² is (C₁-C₂₀)alkyl, cyclo(C₃-C₈)alkyl, cyclo(C₃-C₈)alkenyl, cyclo(C₃-C₈) alkyl(C₁-C₁₀)alkyl, cyclo(C₃-C₈)alkyl(C₂-C₁₀)alkenyl, cyclo(C₃-C₈)alkyl(C₂-C₁₀) alkynyl, cyclo(C₃-C₈)alkenyl(C₁-C₁₀)alkyl, cyclo(C₃-C₈)alkenyl(C₂-C₁₀)alkenyl, cyclo(C₃-C₈)alkenyl(C₂-C₁₀)alkynyl, carboxy(C₁-C₂₀)alkyl, carboxy(C₂-C₂₀)alkenyl, carboxy(C₂-C₂₀)alkynyl, heterocyclyl, heterocyclyl(C₁-C₁₀)alkyl, heterocyclyl(C₂-C₁₀) alkenyl, heterocyclyl(C₂-C₁₀)alkynyl, (C₁-C₁₀)alkoxy(C₁-C₁₀)alkyl, (C₂-C₂₀) alkenyl, (C₂-C₂₀)alkynyl, or (C₁-C₂₀)alkyl, cyclo(C₃-C₈)alkyl, cyclo(C₃-C₈) alkenyl, cyclo(C₃-C₈)alkyl(C₁-C₁₀)alkyl, cyclo(C₃-C₈)alkyl(C₂-C₁₀)alkenyl, cyclo(C₃-C₈)alkyl(C₂-C₁₀)alkynyl, cyclo(C₃-C₈)alkenyl(C₁-C₁₀)alkyl, cyclo (C₃-C₈)alkenyl(C₂-C₁₀)alkenyl, cyclo(C₃-C₈)alkenyl(C₂-C₁₀)alkynyl, carboxy (C₁-C₂₀)alkyl, carboxy(C₂-C₂₀)alkenyl, carboxy(C₂-C₂₀)alkynyl, heterocyclyl, heterocyclyl(C₁-C₁₀)alkyl, heterocyclyl(C₂-C₁₀)alkenyl, heterocyclyl(C₂-C₁₀) alkynyl, (C₁-C₁₀)alkoxy(C₁-C₁₀)alkyl, (C₂-C₂₀)alkenyl or (C₂-C₂₀)alkynyl substituted with one or more halo, aryl or aryl substituted with one or more substituents independently selected from halo, nitro, cyano, halo(C₁-C₁₀)alkyl, halo(C₂-C₁₀)alkenyl, halo(C₂-C₁₀)alkynyl, (C₁-C₁₀)alkylcarbonyl, (C₂-C₁₀) alkenylcarbonyl, (C₂-C₁₀)alkynylcarbonyl, arylcarbonyl, (C₁-C₁₀)alkylsulfonyl and arylsulfonyl, ar(C₁-C₁₀)alkyl, ar(C₂-C₁₀)alkenyl, ar(C₂-C₁₀)alkynyl, or ar(C₁-C₁₀) alkyl, ar(C₂-C₁₀)alkenyl, ar(C₂-C₁₀)alkynyl substituted with one or more substituents independently selected from halo, (C₁-C₁₀)alkyl, (C₂-C₁₀)alkenyl, (C₂-C₁₀) alkynyl, halo(C₁-C₁₀)alkyl, halo(C₂-C₁₀)alkenyl, halo(C₂-C₁₀)alkynyl, (C₁-C₁₀) alkoxy and halo(C₁-C₁₀)alkoxy, heteroaryl, heteroar(C₁-C₁₀)alkyl, heteroar(C₂-C₁₀)alkenyl, heteroar(C₂-C₁₀)alkynyl, or heteroaryl, heteroar(C₁-C₁₀) alkyl, heteroar(C₂-C₁₀)alkenyl, heteroar(C₂-C₁₀)alkynyl substituted with one or more substituents independently selected from halo, (C₁-C₁₀)alkyl, (C₂-C₁₀) alkenyl, (C₂-C₁₀)alkynyl, halo(C₁-C₁₀)alkyl, halo(C₂-C₁₀)alkenyl, halo(C₂-C₁₀) alkynyl, (C₁-C₁₀)alkoxy and halo(C₁-C₁₀)alkoxy,
Y¹ and Y² are each independently halo, and
L is a hydrogen atom, a sodium atom or a potassium atom.

16. The process of claim 15 wherein
Y¹ is chloro,
Y² is chloro or iodo,
L is a sodium atom or a potassium atom,
R¹² is (C₁-C₂₀)alkyl, ar(C₁-C₁₀)alkyl, aryl, heteroaryl or ar(C₁-C₁₀)alkyl, aryl or heteroaryl substituted with one or more substituents independently selected from halo, (C₁-C₁₀)alkyl, (C₂-C₁₀)alkenyl, (C₂-C₁₀)alkynyl, halo(C₁-C₁₀) alkyl, halo(C₂-C₁₀)alkenyl, halo(C₂-C₁₀)alkynyl, (C₁-C₁₀)alkoxy and halo(C₁-C₁₀)alkoxy,
Z³ is (C₁-C₂₀)alkyl, aryl, ar(C₁-C₁₀)alkyl or aryl or ar(C₁-C₁₀)alkyl substituted with one or more substituents independently selected from halo, (C₁-C₁₀) alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy and halo(C₁-C₁₀)alkoxy, and
R² is a hydrogen atom.

17. The process of claim 16 wherein R¹² is (C₁-C₁₀)alkyl, L is sodium and Z³ is (C₁-C₁₀)alkyl or ar(C₁-C₆)alkyl.

18. The process of any one of claims 12-17 wherein the suitable solvent is an ether, dimethylformamide, dimethylsulfoxide, acetonitrile, a chlorinated solvent or a mixture thereof.

19. The process of claim 18 wherein the solvent is diethyl ether.

20. The process of claim 12 or 15 wherein the suitable base is a metal hydride, a metal alkoxide, an alkali metal hydroxide, a tertiary amine, an alkali metal carbonate, a pyridine or a mixture thereof when L is a hydrogen atom.

21. The process of claim 20 wherein the base is sodium hydride.

22. The process of claim 20 wherein about one equivalent of base is used per equivalent of a compound of formula (VI).

23. The process of claim 12 or 15 wherein the reaction temperature is from about −78° C. to about 100° C.

24. The process of any one of claims 12-17 wherein from about 0.9 to about 1.1 equivalents of a compound of formula (VI) are used per equivalent of a compound of formula (V).

25. A process leading to a compound of formula (II) 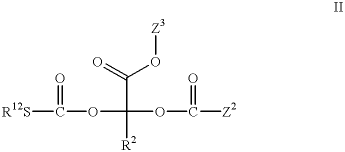
comprising the step of reacting, in the presence of a suitable solvent and a suitable base, a compound of formula (III) 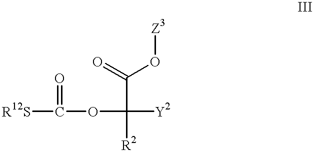
with a compound of formula (IV) 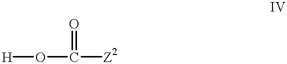
wherein
Z³ is alkyl, alkylcarbonyloxyalkyl, hydroxyalkyl, alkylsulfonylalkyl, acetylaminoalkyl, haloalkyl, alkenyl, acetylaminoalkenyl, haloalkenyl, alkynyl, haloalkynyl, cycloalkyl, cycloalkenyl, carboxycycloalkyl, carboxycycloalkenyl, cycloalkylalkyl, cycloalkylalkenyl, cycloalkenylalkyl, cycloalkenylalkenyl, cycloalkylalkynyl, cycloalkenylalkynyl, carboxycycloalkylalkyl, carboxycycloalkylalkenyl, carboxycycloalkenylalkyl, carboxycycloalkenylalkenyl, carboxycycloalkylalkynyl, carboxycycloalkenylalkynyl, heterocyclyl, heterocyclylalkyl, heterocyclylalkenyl, heterocyclylalkynyl, alkoxyalkyl, alkoxyalkoxyalkyl, alkoxyalkenyl, alkoxyalkynyl, alkoxycarbonylalkyl, alkoxycarbonylalkenyl, alkoxycarbonylalkynyl, haloalkoxyalkyl, haloalkoxyalkenyl, haloalkoxyalkynyl, alkylthioalkyl, alkylthioalkenyl, alkylthioalkynyl, haloalkylthlioalkyl, haloalkylthioalkenyl, haloalkylthioalkynyl, carboxyalkyl, carboxyalkenyl, carboxyalkynyl, SiR³R⁴R⁵, aryl, aryl substituted with one or more substituents independently selected from halo, nitro, hydroxy, cyano, thiocyanato, alkyl, alkylsulfonylalkyl, alkenyl, alkynyl, haloalkyl, haloalkenyl, haloalkynyl, alkoxy, haloalkoxy, SO₂NR³R⁴ and NR³R⁴, aralkyl, aralkenyl, aralkynyl, arcycloalkyl, aroxyalkyl, or aralkyl, aralkenyl, aralkynyl, arcycloalkyl, aroxyalkyl substituted with one or more substituents independently selected from halo, nitro, hydroxy, cyano, alkyl, cycloalkyl, alkenyl, alkynyl, haloalkyl, haloalkenyl, haloalkynyl, alkoxy, haloalkoxy, SO₂NR³R⁴ and NR³R⁴, heteroaryl, heteroaryl substituted with one or more substituents independently selected from halo, nitro, hydroxy, cyano, alkyl, alkenyl, alkynyl, haloalkyl, haloalkenyl, haloalkynyl, alkoxy, haloalkoxy, SO₂NR³R⁴ and NR³R⁴, heteroaralkyl, heteroaralkenyl, heteroaralkynyl, or heteroaralkyl, heteroaralkenyl, heteroaralkynyl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴,
Z² is a hydrogen atom, alkyl, alkylcarbonyloxyalkyl, alkylcarbonyl, alkenylcarbonyl, alkynylcarbonyl, hydroxyalkyl, alkylsulfonylalkyl, acetylaminoalkyl, haloalkyl, alkenyl, acetylaminoalkenyl, haloalkenyl, alkynyl, haloalkynyl, cycloalkyl, cycloalkenyl, carboxycycloalkyl, carboxycycloalkenyl, cycloalkylalkyl, cycloalkylalkenyl, cycloalkenylalkyl, cycloalkenylalkenyl, cycloalkylalkynyl, cycloalkenylalkynyl, carboxycycloalkylalkyl, carboxycycloalkylalkenyl, carboxycycloalkenylalkyl, carboxycycloalkenylalkenyl, carboxycycloalkylalkynyl, carboxycycloalkenylalkynyl, heterocyclyl, heterocyclylalkyl, heterocyclylalkenyl, heterocyclylalkynyl, alkoxyalkyl, alkoxyalkoxyalkyl, alkoxyalkenyl, alkoxyalkynyl, alkoxycarbonylalkyl, alkoxycarbonylalkenyl, alkoxycarbonylalkynyl, haloalkoxyalkyl, haloalkoxyalkenyl, haloalkoxyalkynyl, alkylthioalkyl, alkylthlioalkenyl, alkylthioalkynyl, haloalkylthioalkyl, haloalkylthioalkenyl, haloalkylthioalkynyl, NR³R⁴, SO₂NR³R⁴, carboxyalkyl, carboxyalkenyl, carboxyalkynyl, dialkoxyphosphorylalkyl, aryl, aryl substituted with one or more substituents independently selected from halo, nitro, hydroxy, cyano, thiocyanato, alkyl, alkylsulfonylalkyl, alkenyl, alkynyl, haloalkyl, haloalkenyl, haloalkynyl, alkoxy, haloalkoxy, SO₂NR³R⁴ and NR³R⁴, aralkyl, aralkenyl, aralkynyl, arcycloalkyl, aroxyalkyl, or aralkyl, aralkenyl, aralkynyl, arcycloalkyl, aroxyalkyl substituted with one or more substituents independently selected from halo, nitro, hydroxy, cyano, alkyl, cycloalkyl, alkenyl, alkynyl, haloalkyl, haloalkenyl, haloalkynyl, alkoxy, haloalkoxy, SO₂NR³R⁴ and NR³R⁴, heteroaryl, heteroaryl substituted with one or more substituents independently selected from halo, nitro, hydroxy, cyano, alkyl, alkenyl, alkynyl, haloalkyl, haloalkenyl, haloalkynyl, alkoxy, haloalkoxy, SO₂NR³R⁴ and NR³R⁴, heteroaralkyl, heteroaralkenyl, heteroaralkynyl, or heteroaralkyl, heteroaralkenyl, heteroaralkynyl substituted with one or more substituents independently selected from halo, hydroxy, nitro, cyano, alkyl, alkenyl, alkynyl, haloalkyl, haloalkenyl, haloalkynyl, alkoxy, haloalkoxy, SO₂NR³R⁴ and NR³R⁴, alkylcarbonylalkyl, alkenylcarbonylalkyl, alkynylcarbonylalkyl, heterocyclylcarbonyl, heterocyclylcarbonylalkyl, heterocyclyloxycarbonylalkyl, arylcarbonyl, arylcarbonylalkyl, aralkylcarbonyl, aralkylcarbonylalkyl, aroxycarbonylalkyl, aralkoxycarbonylalkyl, heteroarylcarbonyl, heteroarylcarbonylalkyl, heteroaroxycarbonylalkyl, or arylcarbonyl, arylcarbonylalkyl, aralkylcarbonyl, aralkylcarbonylalkyl, aroxycarbonylalkyl, aralkoxycarbonylalkyl, heteroarylcarbonyl, heteroarylcarbonylalkyl, heteroaroxycarbonylalkyl substituted with one or more substituents independently selected from halo, hydroxy, nitro, cyano, alkyl, alkenyl, alkynyl, haloalkyl, haloalkenyl, haloalkynyl, alkoxy, haloalkoxy, SO₂NR³R⁴ and NR³R⁴, and C(═N—G²²)R²,
G²² is OR³, OCOR³, S(O)ⱼR³, OS(O)ⱼR³, NR³R⁴, OSO₂NR³R⁴, OP(═O)OR³NR³R⁴, OP(═O)(OR³)₂ or N═CR³R⁴,
j is 0, 1 or 2,
R² is a hydrogen atom, alkyl, alkenyl, alkynyl, alkoxyalkyl, alkoxyalkenyl, alkoxyalkynyl, alkylthioalkyl, alkylthioalkenyl, alkylthioalkynyl, carboxy, a carboxylate salt, carboxyalkyl, carboxyalkenyl, carboxyalkynyl, alkoxycarbonyl, alkoxycarbonylalkyl, alkoxycarbonylalkenyl, alkoxycarbonylalkynyl, alkylcarbonyl, alkenylcarbonyl, alkynylcarbonyl, cycloalkyl, cycloalkenyl, cycloalkylalkyl, cycloalkenylalkyl, cycloalkylalkenyl, cycloalkenylalkenyl, cycloalkylalkynyl, cycloalkenylalkynyl, heterocyclyl, heterocyclylalkyl, heterocyclylalkenyl, heterocyclylalkynyl, or alkyl, alkenyl, alkynyl, alkoxyalkyl, alkoxyalkenyl, alkoxyalkynyl, alkylthioalkyl, alkylthioalkenyl, alkylthioalkynyl, carboxyalkyl, carboxyalkenyl, carboxyalkynyl, alkoxycarbonyl, alkoxycarbonylalkyl, alkoxycarbonylalkenyl, alkoxycarbonylalkynyl, alkylcarbonyl, alkenylcarbonyl, alkynylcarbonyl, cycloalkyl, cycloalkenyl, cycloalkylalkyl, cycloalkenylalkyl, cycloalkylalkenyl, cycloalkenylalkenyl, cycloalkylalkynyl, cycloalkenylalkynyl, heterocyclyl, heterocyclylalkyl, heterocyclylalkenyl, heterocyclylalkynyl substituted with one or more substituents independently selected from halo, cyano, hydroxy, nitro, SO₂NR³R⁴ and NR³R⁴, aryl or aryl substituted with one or more substituents independently selected from halo, hydroxy, cyano, nitro, alkyl, alkenyl, alkynyl, haloalkyl, haloalkenyl, haloalkynyl, alkoxy, haloalkoxy, carboxy, alkoxycarbonyl, SO₂NR³R⁴ and NR³R⁴, aralkyl, aralkenyl, aralkynyl or aralkyl, aralkenyl, aralkynyl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, arylcarbonyl, aralkylcarbonyl, aralkenylcarbonyl, aralkynylcarbonyl or arylcarbonyl, aralkylcarbonyl, aralkenylcarbonyl, aralkynylcarbonyl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, heteroaryl or heteroaryl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, heteroaralkyl, heteroaralkenyl, heteroaralkynyl or heteroaralkyl, heteroaralkenyl, heteroaralkynyl substituted with one or more substituents independently selected from halo, cyano, hydroxy, nitro, alkyl, alkenyl, alkynyl, haloalkyl, haloalkenyl, haloalkynyl, alkoxy, haloalkoxy, SO₂NR³R⁴ and NR³R⁴, heteroaralkylcarbonyl, heteroaralkenylcarbonyl, heteroaralkynylcarbonyl or heteroaralkylcarbonyl, heteroaralkenylcarbonyl, heteroaralkynylcarbonyl substituted with one or more substituents independently selected from halo, cyano, hydroxy, nitro, alkyl, alkenyl, alkynyl, haloalkyl, haloalkenyl, haloalkynyl, alkoxy, haloalkoxy, SO₂NR³R⁴ and NR³R⁴,
Y² is halo,
R¹² is alkyl, cycloalkyl, cycloalkenyl, cycloalkylalkyl, cycloalkylalkenyl, cycloalkylalkynyl, cycloalkenylalkyl, cycloalkenylalkenyl, cycloalkenylalkynyl, carboxyalkyl, carboxyalkenyl, carboxyalkynyl, heterocyclyl, heterocyclylalkyl, heterocyclylalkenyl, heterocyclylalkynyl, alkoxyalkyl, alkenyl, alkynyl, or alkyl, cycloalkyl, cycloalkenyl, cycloalkylalkyl, cycloalkylalkenyl, cycloalkylalkynyl, cycloalkenylalkyl, cycloalkenylalkenyl, cycloalkenylalkynyl, carboxyalkyl, carboxyalkenyl, carboxyalkynyl, heterocyclyl, heterocyclylalkyl, heterocyclylalkenyl, heterocyclylalkynyl, alkoxyalkyl, alkenyl or alkynyl substituted with one or more halo, aryl or aryl substituted with one or more substituents independently selected from halo, nitro, cyano, haloalkyl, haloalkenyl, haloalkynyl, alkylcarbonyl, alkenylcarbonyl, alkynylcarbonyl, arylcarbonyl, alkylsulfonyl and arylsulfonyl, aralkyl, aralkenyl, aralkynyl, or aralkyl, aralkenyl, aralkynyl substituted with one or more substituents independently selected from halo, alkyl, alkenyl, alkynyl, haloalkyl, haloalkenyl, haloalkynyl, alkoxy and haloalkoxy, heteroaryl, heteroaralkyl, heteroaralkenyl, heteroaralkynyl, or heteroaryl, heteroaralkyl, heteroaralkenyl, heteroaralkynyl substituted with one or more substituents independently selected from halo, alkyl, alkenyl, alkynyl, haloalkyl, haloalkenyl, haloalkynyl, alkoxy and haloalkoxy,
R³, R⁴ and R⁵ are each independently a hydrogen atom, alkyl, cycloalkyl, cycloalkenyl, cycloalkylalkyl, cycloalkylalkenyl, cycloalkylalkynyl, cycloalkenylalkyl, cycloalkenylalkenyl, cycloalkenylalkynyl, carboxyalkyl, carboxyalkenyl, carboxyalkynyl, heterocyclyl, heterocyclylalkyl, heterocyclylalkenyl, heterocyclylalkynyl, alkoxyalkyl, alkenyl, alkynyl, or alkyl, cycloalkyl, cycloalkenyl, cycloalkylalkyl, cycloalkylalkenyl, cycloalkylalkynyl, cycloalkenylalkyl, cycloalkenylalkenyl, cycloalkenylalkynyl, carboxyalkyl, carboxyalkenyl, carboxyalkynyl, heterocyclyl, heterocyclylalkyl, heterocyclylalkenyl, heterocyclylalkynyl, alkoxyalkyl, alkenyl or alkynyl substituted with one or more halo, aryl, aralkyl, aralkenyl, aralkynyl, or aryl, aralkyl, aralkenyl, aralkynyl substituted with one or more substituents independently selected from halo, alkyl, alkenyl, alkynyl, haloalkyl, haloalkenyl, haloalkynyl, alkoxy and haloalkoxy, heteroaryl, heteroaralkyl, heteroaralkenyl, heteroaralkynyl, or heteroaryl, heteroaralkyl, heteroaralkenyl, heteroaralkynyl substituted with one or more substituents independently selected from halo, alkyl, alkenyl, alkynyl, haloalkyl, haloalkenyl, haloalkynyl, alkoxy and haloalkoxy, or R³ and R⁴ taken together with the nitrogen atom to which they are attached form a 5-or 6-membered saturated or unsaturated heterocyclic ring.

26. The process of claim 25 wherein
Y² is chloro, bromo or iodo,
R¹² is alkyl, aralkyl, aryl, heteroaryl, or aralkyl, aryl or heteroaryl substituted with one or more substituents independently selected from halo, alkyl, alkenyl, alkynyl, haloalkyl, haloalkenyl, haloalkynyl, alkoxy and haloalkoxy,
Z³ is alkyl, aryl, aralkyl or aryl or aralkyl substituted with one or more substituents independently selected from halo, alkyl, alkenyl, alkynyl, haloalkyl, haloalkenyl, haloalkynyl, alkoxy and haloalkoxy, and
R² is a hydrogen atom.

27. The process of claim 26 wherein Y² is chloro or iodo, R¹² is alkyl and Z³ is alkyl or aralkyl.

28. The process of claim 25 wherein
Z³ is (C₁-C₂₀)alkyl, (C₁-C₁₀)alkylcarbonyloxy(C₁-C₁₀)alkyl, hydroxy (C₁-C₂₀)alkyl, (C₁-C₁₀)alkylsulfonyl(C₁-C₁₀)alkyl, acetylamino(C₁-C₁₀)alkyl, halo (C₁-C₂₀)alkyl, (C₂-C₁₀)alkenyl, acetylamino(C₂-C₁₀)alkenyl, halo(C₂-C₁₀)alkenyl, (C₂-C₁₀)alkynyl, halo(C₂-C₁₀)alkynyl, cyclo(C₃-C₈)alkyl, cycoI(C₃-C₈)alkenyl, carboxycyclo(C₃-C₈)alkyl, carboxycyclo(C₃-C₈)alkenyl, cyclo(C₃-C₈)alkyl(C₁-C₁₀) alkyl, cyclo(C³-C₈)alkyl(C₂-C₁₀)alkenyl, cyclo(C₃-C₈)alkenyl(C₁-C₁₀)alkyl, cyclo(C₃-C₈)alkenyl(C₂-C₁₀)alkenyl, cyclo(C₃-C₈)alkyl(C₂-C₁₀)alkynyl, cyclo (C₃-C₈)alkenyl(C₂-C₁₀)alkynyl, carboxycyclo(C₃-C₈)alkyl(C₁-C₁₀)alkyl, carboxycyclo(C₃-C₈)alkyl(C₂-C₁₀)alkenyl, carboxycyclo(C₃-C₈)alkenyl(C₁-C₁₀)alkyl, carboxycyclo(C₃-C₈)alkenyl(C₂-C₁₀)alkenyl, carboxycyclo(C₃-C₈)alkyl(C₂-C₁₀) alkynyl, carboxycyclo(C₃-C₈)alkenyl(C₂-C₁₀)alkynyl, heterocyclyl, heterocyclyl(C₁-C₁₀)alkyl, heterocyclyl(C₂-C₁₀)alkenyl, heterocyclyl(C₂-C₁₀) alkynyl, (C₁-C₁₀)alkoxy(C₁-C₁₀)alkyl, (C₁-C₁₀)alkoxy(C₁-C₁₀)alkoxy(C₁-C₁₀) alkyl, (C₁-C₁₀)alkoxy(C₂-C₁₀)alkenyl, (C₁-C₁₀)alkoxy(C₂-C₁₀)alkynyl, (C₁-C₁₀) alkoxycarbonyl(C₁-C₁₀)alkyl, (C₁-C₁₀)alkoxycarbonyl(C₂-C₁₀)alkenyl, (C₁-C₁₀) alkoxycarbonyl(C₂-C₁₀)alkynyl, halo(C₁-C₁₀)alkoxy(C₁-C₁₀)alkyl, halo(C₁-C₁₀) alkoxy(C₂-C₁₀)alkenyl, halo(C₁-C₁₀)alkoxy(C₂-C₁₀)alkynyl, (C₁-C₁₀) alkylthio(C₁-C₁₀)alkyl, (C₁-C₁₀)alkylthio(C₂-C₁₀)alkenyl, (C₁-C₁₀)alkylthio (C₂-C₁₀)alkynyl, halo(C₁-C₁₀)alkylthio(C₁-C₁₀)alkyl, halo(C₁-C₁₀)alkylthio(C₂-C₁₀) alkenyl, halo(C₁-C₁₀)alkylthio(C₂-C₁₀)alkynyl, carboxy(C₁-C₂₀)alkyl, carboxy(C₂-C₁₀)alkenyl, carboxy(C₂-C₁₀)alkynyl, SiR³R⁴R⁵, aryl, aryl substituted with one or more substituents independently selected from halo, nitro, hydroxy, cyano, thiocyanato, (C₁-C₁₀)alkyl, (C₁-C₁₀)alkylsulfonyl(C₁-C₁₀)alkyl, (C₂-C₁₀) alkenyl, (C₂-C₁₀)alkynyl, halo(C₁-C₁₀)alkyl, halo(C₂-C₁₀)alkenyl, halo(C₂-C₁₀) alkynyl, (C₁-C₁₀)alkoxy, halo(C₁-C₁₀)alkoxy, SO₂NR³R⁴ and NR³R⁴, ar(C₁-C₁₀) alkyl, ar(C₂-C₁₀)alkenyl, ar(C₂-C₁₀)alkynyl, arcyclo(C₃-C₈)alkyl, aroxy(C₁-C₁₀) alkyl, or ar(C₁-C₁₀)alkyl, ar(C₂-C₁₀)alkenyl, ar(C₂-C₁₀)alkynyl, arcyclo(C₃-C₈) alkyl, aroxy(C₁-C₁₀)alkyl substituted with one or more substituents independently selected from halo, nitro, hydroxy, cyano, (C₁-C₁₀)alkyl, cycloalkyl, (C₂-C₁₀)alkenyl, (C₂-C₁₀)alkynyl, halo(C₁-C₁₀)alkyl, halo(C₂-C₁₀)alkenyl, halo(C₂-C₁₀) alkynyl, (C₁-C₁₀)alkoxy, halo(C₁-C₁₀)alkoxy, SO₂NR³R⁴ and NR³R⁴, heteroaryl, heteroaryl substituted with one or more substituents independently selected from halo, nitro, hydroxy, cyano, (C₁-C₁₀)alkyl, (C₂-C₁₀)alkenyl, (C₂-C₁₀) alkynyl, halo(C₁-C₁₀)alkyl, halo(C₂-C₁₀)alkenyl, halo(C₂-C₁₀)alkynyl, (C₁-C₁₀) alkoxy, halo(C₁-C₁₀)alkoxy, SO₂NR³R⁴ and NR³R⁴, heteroar(C₁-C₁₀)alkyl, heteroar(C₂-C₁₀)alkenyl, heteroar(C₂-C₁₀)alkynyl, or heteroar(C₁-C₁₀)alkyl, heteroar(C₂-C₁₀)alkenyl, heteroar(C₂-C₁₀)alkynyl substituted with one or more substituents independently selected from halo, hydroxy, cyano, nitro, (C₁-C₁₀) alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴,
Z² is a hydrogen atom, (C₁-C₂₀)alkyl, (C₁-C₁₀)alkylcarbonyloxy(C₁-C₁₀) alkyl, (C₁-C₂₀)alkylcarbonyl, (C₁-C₂₀)alkenylcarbonyl, (C₁-C₂₀) alkynylcarbonyl, hydroxy(C₁-C₂₀)alkyl, (C₁-C₁₀)alkylsulfonyl(C₁-C₁₀)alkyl, acetylamino(C₁-C₁₀)alkyl, halo (C₁-C₂₀)alkyl, (C₂-C₂₀)alkenyl, halo(C₂-C₂₀)alkenyl, acetylamino(C₂-C₁₀)alkenyl, (C₂-C₂₀)alkynyl, halo(C₂-C₂₀)alkynyl, cyclo(C₃-C₈) alkyl, cyclo(C₃-C₈)alkenyl, carboxycyclo(C₃-C₈)alkyl, carboxycyclo(C₃-C₈) alkenyl, cyclo(C₃-C₈)alkyl(C₁-C₁₀)alkyl, cyclo(C₃-C₈)alkyl(C₂-C₁₀)alkenyl, cyclo(C₃-C₈)alkenyl(C₁-C₁₀)alkyl, cyclo(C₃-C₈)alkenyl(C₂-C₁₀)alkenyl, cyclo (C₃-C₈)alkyl(C₂-C₁₀)alkynyl, cyclo(C₃-C₈)alkenyl(C₂-C₁₀)alkynyl, carboxycyclo (C₃-C₈)alkyl(C₁-C₁₀)alkyl, carboxy(C₃-C₈)cycloalkyl(C₂-C₁₀)alkenyl, carboxycyclo (C₃-C₈)alkenyl(C₁-C₁₀)alkyl, carboxycyclo(C₃-C₈)alkenyl(C₂-C₁₀)alkenyl, carboxycyclo(C₃-C₈)alkyl(C₂-C₁₀)alkynyl, carboxycyclo(C₃-C₈)alkenyl(C₂-C₁₀) alkynyl, heterocyclyl, heterocyclyl(C₁-C₁₀)alkyl, heterocyclyl(C₂-C₁₀)alkenyl, heterocyclyl(C₂-C₁₀)alkynyl, (C₁-C₁₀)alkoxy(C₁-C₁₀)alkyl, (C₁-C₅)alkoxy (C₁-C₅)alkoxy(C₁-C₁₀)alkyl, (C₁-C₁₀)alkoxy(C₂-C₁₀)alkenyl, (C₁-C₁₀)alkoxy (C₂-C₁₀)alkynyl, (C₁-C₁₀)alkoxycarbonyl(C₁C₁₀)alkyl, (C₁-C₁₀)alkoxycarbonyl (C₂-C₁₀)alkenyl, (C₁-C₁₀)alkoxycarbonyl(C₂-C₁₀)alkynyl, halo(C₁-C₁₀)alkoxy (C₁-C₁₀)alkyl, halo(C₁-C₁₀)alkoxy(C₂-C₁₀)alkenyl, halo(C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀)alkylthio(C₂-C₁₀)alkynyl, halo(C₁-C₁₀)alkylthio(C₁-C₁₀)alkyl, halo(C₁-C₁₀) alkylthio(C₂-C₁₀)alkenyl, halo(C₁-C₁₀)alkylthio(C₂-C₁₀)alkynyl, SO₂NR³R⁴, NR³R⁴, carboxy(C₁-C₂₀)alkyl, carboxy(C₂-C₂₀)alkenyl, carboxy(C₂-C₂₀)alkynyl, di(C₁-C₁₀)alkoxyphosphoryl(C₁-C₁₀)alkyl, aryl, aryl substituted with one or more substituents independently selected from halo, nitro, cyano, hydroxy, (C₁-C₁₀) alkyl, (C₁-C₁₀)alkylsulfonyl(C₁-C₁₀)alkyl, (C₁-C₁₀)alkylsulfonyl, thiocyanato, (C₂-C₁₀)alkenyl, (C₂-C₁₀)alkynyl, halo(C₁-C₁₀)alkyl, halo(C₂-C₁₀)alkenyl, halo (C₂-C₁₀)alkynyl, (C₁-C₁₀)alkoxy, halo(C₁-C₁₀)alkoxy, SO₂NR³R⁴, and NR³R⁴, ar (C₁-C₁₀)alkyl, ar(C₂-C₁₀)alkenyl, ar(C₂-C₁₀)alkynyl, arcyclo(C₃-C₈)alkyl, aroxy(C₁-C₁₀) alkyl, or ar(C₁-C₁₀)alkyl, ar(C₂-C₁₀)alkenyl, ar(C₂-C₁₀)alkynyl, arcyclo(C₃-C₈) alkyl, aroxy(C₁-C₁₀)alkyl substituted with one or more substituents independently selected from halo, nitro, hydroxy, cyano, (C₁-C₁₀)alkyl, cyclo(C₃-C₈) alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroaryl, heteroaryl substituted with one or more substituents independently selected from halo, hydroxy, nitro, cyano, (C₁-C₁₀)alkyl, (C₂-C₁₀)alkenyl, (C₂-C₁₀) alkynyl, halo(C₁-C₁₀)alkyl, halo(C₂-C₁₀)alkenyl, halo(C₂-C₁₀)alkynyl, (C₁-C₁₀) alkoxy, halo(C₁-C₁₀)alkoxy and NR³R⁴, heteroar(C₁-C₁₀)alkyl, heteroar(C₂-C₁₀) alkenyl, heteroar(C₂-C₁₀)alkynyl, or heteroar(C₁-C₁₀)alkyl, heteroar(C₂-C₁₀) alkenyl, heteroar(C₂-C₁₀)alkynyl substituted with one or more substituents independently selected from halo, hydroxy, cyano, nitro, (C₁-C₁₀)alkyl, (C₂-C₁₀) alkenyl, (C₂-C₁₀)alkynyl, halo(C₁-C₁₀)alkyl, halo(C₂-C₁₀)alkenyl, halo(C₂-C₁₀) alkynyl, (C₁-C₁₀)alkoxy, halo(C₁-C₁₀)alkoxy, SO₂NR³R⁴ and NR³R⁴, (C₁-C₁₀)alkylcarbonyl(C₁-C₁₀)alkyl, (C₂-C₁₀)alkenylcarbonyl(C₁-C₁₀)alkyl, (C₂-C₁₀)alkynylcarbonyl(C₁-C₁₀)alkyl, heterocyclylcarbonyl, heterocyclylcarbonyl(C₁-C₁₀) alkyl, heterocyclyloxycarbonyl(C₁-C₁₀)alkyl, arylcarbonyl, arylcarbonyl(C₁-C₁₀) alkyl, ar(C₁-C₁₀)alkylcarbonyl, ar(C₁-C₁₀)alkylcarbonyl(C₁-C₁₀)alkyl, aroxycarbonyl(C₁-C₁₀)alkyl, ar(C₁-C₁₀)alkoxycarbonyl(C₁-C₁₀)alkyl, heteroarylcarbonyl, heteroarylcarbonyl(C₁-C₁₀)alkyl, heteroaroxycarbonyl(C₁-C₁₀) alkyl, or arylcarbonyl, arylcarbonyl(C₁-C₁₀)alkyl, ar(C₁-C₁₀)alkylcarbonyl, ar(C₁-C₁₀)alkylcarbonyl(C₁-C₁₀)alkyl, aroxycarbonyl(C₁-C₁₀)alkyl, ar (C₁-C₁₀)alkoxycarbonyl(C₁-C₁₀)alkyl, heteroarylcarbonyl, heteroarylcarbonyl (C₁-C₁₀)alkyl, heteroaroxycarbonyl(C₁-C₁₀)alkyl substituted with one or more substituents independently selected from halo, hydroxy, nitro, cyano, (C₁-C₁₀) alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, and C(═N—G²²)R²,
G²² is OR³, OCOR³, S(O)ⱼR³, OS(O)ⱼR³, NR³R⁴, OSO₂NR³R⁴, OP(═O)OR³NR³R⁴, OP(═O)(OR³)₂ or N═CR³R⁴,
j is 0, 1 or 2,
R² is a hydrogen atom, (C₁-C₂₀)alkyl, (C₂-C₁₀)alkenyl, (C₂-C₁₀)alkynyl, (C₁-C₁₀) alkoxy(C₁-C₁₀)alkyl, (C₁-C₁₀)alkoxy(C₂-C₁₀)alkenyl, (C₁-C₁₀)alkoxy(C₂-C₁₀) alkynyl, (C₁-C₁₀)alkylthio(C₁-C₁₀)alkyl, (C₁-C₁₀)alkylthio(C₂-C₁₀)alkenyl, (C₁-C₁₀) alkylthio(C₂-C₁₀)alkynyl, carboxy, a carboxylate salt, carboxy(C₁-C₂₀)alkyl, carboxy(C₂-C₂₀)alkenyl, carboxy(C₂-C₂₀)alkynyl, (C₁-C₂₀)alkoxycarbonyl, (C₁-C₁₀) alkoxycarbonyl(C₁-C₁₀)alkyl, (C₁-C₁₀)alkoxycarbonyl(C₂-C₁₀)alkenyl, (C₁-C₁₀) alkoxycarbonyl(C₂-C₁₀)alkynyl, (C₁-C₂₀)alkylcarbonyl, (C₂-C₂₀) alkenylcarbonyl, (C₂-C₂₀)alkynylcarbonyl, cyclo(C₃-C₈)alkyl, cyclo(C₃-C₈) alkenyl, cyclo(C₃-C₈)alkyl(C₁-C₁₀)alkyl, cyclo(C₃-C₈)alkenyl(C₁-C₁₀)alkyl, cyclo(C₃-C₈)alkyl(C₂-C₁₀)alkenyl, cyclo(C₃-C₈)alkenyl(C₂-C₁₀)alkenyl, cyclo(C₃-C₈) alkyl(C₂-C₁₀)alkynyl, cyclo(C₃-C₈)alkenyl(C₂-C₁₀)alkynyl, heterocyclyl, heterocyclyl(C₁-C₁₀)alkyl, heterocyclyl(C₂-C₁₀)alkenyl, heterocyclyl(C₂-C₁₀) alkynyl, or (C₁-C₂₀)alkyl, (C₂-C₁₀)alkenyl, (C₂-C₁₀)alkynyl, (C₁-C₁₀)alkoxy(C₁-C₁₀) alkyl, (C₁-C₁₀)alkoxy(C₂-C₁₀)alkenyl, (C₁-C₁₀)alkoxy(C₂-C₁₀)alkynyl, (C₁-C₁₀) alkylthio(C₁-C₁₀)alkyl, (C₁-C₁₀)alkylthio(C₂-C₁₀)alkenyl, (C₁₀-C₁₀)alkylthio(C₂-C₁₀) alkynyl, carboxy(C₁-C₂₀)alkyl, carboxy(C₂-C₁₀)alkenyl, carboxy(C₂-C₁₀) alkynyl, (C₁-C₂₀)alkoxycarbonyl, (C₁-C₁₀)alkoxycarbonyl(C₁-C₁₀)alkyl, (C₁-C₁₀) alkoxycarbonyl(C₂-C₁₀)alkenyl, (C₁-C₁₀)alkoxycarbonyl(C₂-C₁₀)alkynyl, (C₁-C₂₀) alkylcarbonyl, (C₂-C₁₀)alkenylcarbonyl, (C₂-C₁₀)alkynylcarbonyl, cyclo(C₃-C₈)alkyl, cyclo (C₃-C₈)alkenyl, cyclo(C₃-C₈)alkyl(C₁-C₁₀)alkyl, cyclo(C₃-C₈) alkenyl(C₁-C₁₀)alkyl, cyclo(C₃-C₈)alkyl(C₂-C₁₀)alkenyl, cyclo(C₃-C₈)alkenyl (C₂-C₁₀)alkenyl, cyclo(C₃-C₈)alkyl(C₂-C₁₀)alkynyl, cyclo(C₃-C₈)alkenyl(C₂-C₁₀)alkynyl, heterocyclyl, heterocyclyl(C₁-C₁₀)alkyl, heterocyclyl(C₂-C₁₀)alkenyl, heterocyclyl(C₂-C₁₀)alkynyl substituted with one or more substituents independently selected from halo, cyano, hydroxy, nitro, SO₂NR³R⁴ and NR³R⁴, aryl or aryl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, carboxy, (C₁-C₄)alkoxycarbonyl, SO₂NR³R⁴ and NR³R⁴, ar(C₁-C₁₀)alkyl, ar (C₂-C₁₀)alkenyl, ar(C₂-C₁₀)alkynyl, or ar(C₁-C₁₀)alkyl, ar(C₂-C₁₀)alkenyl, ar (C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo (C₁-C₁₀)alkoxy, SO₂NR³R⁴ and NR³R⁴, arylcarbonyl, ar(C₁-C₁₀)alkylcarbonyl, ar(C₂-C₁₀) alkenylcarbonyl, ar(C₂-C₁₀)alkynylcarbonyl or arylcarbonyl, ar(C₁-C₁₀) alkylcarbonyl, ar(C₂-C₁₀)alkenylcarbonyl, ar(C₂-C₁₀)alkynylcarbonyl substituted with one or more substituents independently selected from halo, hydroxy, cyano, nitro, (C₁-C₁₀)alkyl, (C₂-C₁₀)alkenyl, (C₂-C₁₀)alkynyl, halo (C₁-C₁₀)alkyl, halo(C₂-C₁₀)alkenyl, halo(C₂-C₁₀)alkynyl, (C₁-C₁₀)alkoxy, halo (C₁-C₁₀)alkoxy, SO₂NR³R⁴ and NR³R⁴, heteroaryl, heteroaryl substituted with one or more substituents independently selected from halo, (C₁-C₁₀)alkyl, (C₂-C₁₀) alkenyl, (C₂-C₁₀)alkynyl, halo(C₁-C₁₀)alkyl, halo(C₂-C₁₀)alkenyl, halo(C₂-C₁₀) alkynyl, (C₁-C₁₀)alkoxy, halo(C₁-C₁₀)alkoxy and NR³R⁴, heteroar(C₁-C₁₀)alkyl, heteroar(C₂-C₁₀)alkenyl, heteroar(C₂-C₁₀)alkynyl or heteroar(C₁-C₁₀)alkyl, heteroar(C₂-C₁₀)alkenyl, heteroar(C₂-C₁₀)alkynyl substituted with one or more substituents independently selected from halo, (C₁-C₁₀)alkyl, (C₂-C₁₀)alkenyl, (C₂- C₁₀)alkynyl, halo(C₁-C₁₀)alkyl, halo(C₂-C₁₀)alkenyl, halo(C₂-C₁₀)alkynyl, (C₁- C₁₀)alkoxy, halo(C₁-C₁₀)alkoxy, SO₂NR³R⁴ and NR³R⁴, heteroar(C₁- C₁₀)alkylcarbonyl, heteroar(C₂-C₁₀)alkenylcarbonyl, heteroar(C₂-C₁₀) alkynylcarbonyl or heteroar(C₁-C₁₀)alkylcarbonyl, heteroar(C₂-C₁₀) alkenylcarbonyl, heteroar(C₂-C₁₀)alkynylcarbonyl substituted with one or more substituents independently selected from halo, cyano, hydroxy, nitro, (C₁-C₁₀) alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, or R¹ and R² taken together with the carbon atom to which they are attached form a 5-7 membered saturated or unsaturated ring,
R³, R⁴ and R⁵ are each independently a hydrogen atom, (C₁-C₂₀)alkyl, cyclo(C₃-C₈)alkyl, cyclo(C₃-C₈)alkenyl, cyclo(C₃-C₈)alkyl(C₁-C₁₀)alkyl, cyclo (C₃-C₈)alkyl(C₂-C₁₀)alkenyl, cyclo(C₃-C₈)alkyl(C₂-C₁₀)alkynyl, cyclo(C₃-C₈)alkenyl (C₁-C₁₀)alkyl, cyclo(C₃-C₈)alkenyl(C₂-C₁₀)alkenyl, cyclo(C₃-C₈)alkenyl(C₂-C₁₀)alkynyl, carboxy(C₁-C₂₀)alkyl, carboxy(C₂-C₁₀)alkenyl, carboxy(C₂-C₁₀)alkynyl, heterocyclyl, heterocyclyl(C₁-C₁₀)alkyl, heterocyclyl(C₂-C₁₀)alkenyl, heterocyclyl(C₂-C₁₀)alkynyl, (C₁-C₁₀)alkoxy(C₁-C₁₀)alkyl, (C₂-C₁₀)alkenyl, (C₂-C₁₀) alkynyl, or (C₁-C₁₀)alkyl, cyclo(C₃-C₈)alkyl, cyclo(C₃-C₈)alkenyl, cyclo(C₃-C₈) alkyl(C₁-C₁₀)alkyl, cyclo(C₃-C₈)alkyl(C₂-C₁₀)alkenyl, cyclo(C₃-C₈)alkyl(C₂-C₁₀) alkynyl, cyclo(C₃-C₈)alkenyl(C₁-C₁₀)alkyl, cyclo(C₃-C₈)alkenyl(C₂-C₁₀)alkenyl, cyclo(C₃-C₈)alkenyl(C₂-C₁₀)alkynyl, carboxy(C₁-C₂₀)alkyl, carboxy(C₂-C₁₀)alkenyl, carboxy(C₂-C₁₀)alkynyl, heterocyclyl, heterocyclyl(C₁-C₁₀)alkyl, heterocyclyl(C₂-C₁₀) alkenyl, heterocyclyl(C₂-C₁₀)alkynyl, (C₁-C₁₀)alkoxy(C₁-C₁₀)alkyl, (C₂-C₁₀) alkenyl or (C₂-C₁₀)alkynyl substituted with one or more halo, aryl, ar(C₁-C₁₀) alkyl, ar(C₂-C₁₀)alkenyl, ar(C₂-C₁₀)alkynyl or aryl, ar(C₁-C₁₀)alkyl, ar(C₂-C₁₀) alkenyl, ar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy and halo(C₁-C₁₀)alkoxy, heteroaryl, heteroar(C₁-C₁₀)alkyl, heteroar(C₂-C₁₀)alkenyl, heteroar(C₂-C₁₀)alkynyl or heteroaryl, heteroar(C₁-C₁₀)alkyl, heteroar(C₂-C₁₀) alkenyl, heteroar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy and halo(C₁-C₁₀)alkoxy, or R³ and R⁴ taken together with the nitrogen atom to which they are attached form a 5-or 6-membered saturated or unsaturated heterocyclic ring,
R¹² is (C₁-C₂₀)alkyl, cyclo(C₃-C₈)alkyl, cyclo(C₃-C₈)alkenyl, cyclo(C₃-C₈) alkyl(C₁-C₁₀)alkyl, cyclo(C₃-C₈)alkyl(C₂-C₁₀)alkenyl, cyclo(C₃-C₈)alkyl(C₂-C₁₀) alkynyl, cyclo(C₃-C₈)alkenyl(C₁-C₁₀)alkyl, cyclo(C₃-C₈)alkenyl(C₂-C₁₀)alkenyl, cyclo(C₃-C₈)alkenyl(C₂-C₁₀)alkynyl, carboxy(C₁-C₂₀)alkyl, carboxy(C₂-C₂₀)alkenyl, carboxy(C₂-C₂₀)alkynyl, heterocyclyl, heterocyclyl(C₁-C₁₀)alkyl, heterocyclyl(C₂-C₁₀) alkenyl, heterocyclyl(C₂-C₁₀)alkynyl, (C₁-C₁₀)alkoxy(C₁-C₁₀)alkyl, (C₂-C₂₀) alkenyl, (C₂-C₂₀)alkynyl, or (C₁-C₂₀)alkyl, cyclo(C₃-C₈)alkyl, cyclo(C₃-C₈) alkenyl, cyclo(C₃-C₈)alkyl(C₁-C₁₀)alkyl, cyclo(C₃-C₈)alkyl(C₂-C₁₀)alkenyl, cyclo(C₃-C₈)alkyl(C₂-C₁₀)alkynyl, cyclo(C₃-C₈)alkenyl(C₁-C₁₀)alkyl, cyclo(C₃-C₈) alkenyl(C₂-C₁₀)alkenyl, cyclo(C₃-C₈)alkenyl(C₂-C₁₀)alkynyl, carboxy(C₁-C₂₀) alkyl, carboxy(C₂-C₂₀)alkenyl, carboxy(C₂-C₂₀)alkynyl, heterocyclyl, heterocyclyl(C₁-C₁₀)alkyl, heterocyclyl(C₂-C₁₀)alkenyl, heterocyclyl(C₂-C₁₀) alkynyl, (C₁-C₁₀)alkoxy(C₁-C₁₀)alkyl, (C₂-C₂₀)alkenyl or (C₂-C₂₀)alkynyl substituted with one or more halo, aryl or aryl substituted with one or more substituents independently selected from halo, nitro, cyano, halo(C₁-C₁₀)alkyl, halo(C₂-C₁₀)alkenyl, halo(C₂-C₁₀)alkynyl, (C₁-C₁₀)alkylcarbonyl, (C₂-C₁₀) alkenylcarbonyl, (C₂-C₁₀)alkynylcarbonyl, arylcarbonyl, (C₁-C₁₀)alkylsulfonyl and arylsulfonyl, ar(C₁-C₁₀)alkyl, ar(C₂-C₁₀)alkenyl, ar(C₂-C₁₀)alkynyl, or ar(C₁-C₁₀) alkyl, ar(C₂-C₁₀)alkenyl, ar(C₂-C₁₀)alkynyl substituted with one or more substituents independently selected from halo, (C₁-C₁₀)alkyl, (C₂-C₁₀)alkenyl, (C₂-C₁₀) alkynyl, halo(C₁-C₁₀)alkyl, halo(C₂-C₁₀)alkenyl, halo(C₂-C₁₀)alkynyl, (C₁-C₁₀) alkoxy and halo(C₁-C₁₀)alkoxy, heteroaryl, heteroar(C₁-C₁₀)alkyl, heteroar(C₂-C₁₀)alkenyl, heteroar(C₂-C₁₀)alkynyl, or heteroaryl, heteroar(C₁-C₁₀) alkyl, heteroar(C₂-C₁₀)alkenyl, heteroar(C₂-C₁₀)alkynyl substituted with one or more substituents independently selected from halo, (C₁-C₁₀)alkyl, (C₂-C₁₀) alkenyl, (C₂-C₁₀)alkynyl, halo(C₁-C₁₀)alkyl, halo(C₂-C₁₀)alkenyl, halo(C₂-C₁₀) alkynyl, (C₁-C₁₀)alkoxy and halo(C₁-C₁₀)alkoxy, and
Y² is chloro, bromo or iodo.

29. The process of claim 28 wherein
Y² is chloro, bromo or iodo,
R¹² is (C₁-C₂₀)alkyl, ar(C₁-C₁₀)alkyl, aryl, heteroaryl or ar(C₁-C₁₀)alkyl, aryl or heteroaryl substituted with one or more substituents independently selected from halo, (C₁-C₁₀)alkyl, (C₂-C₁₀)alkenyl, (C₂-C₁₀)alkynyl, halo(C₁-C₁₀) alkyl, halo(C₂-C₁₀)alkenyl, halo(C₂-C₁₀)alkynyl, (C₁-C₁₀)alkoxy and halo(C₁-C₁₀) alkoxy,
Z³ is (C₁-C₂₀)alkyl, aryl, ar(C₁-C₁₀)alkyl or aryl or ar(C₁-C₁₀)alkyl substituted with one or more substituents independently selected from halo, (C₁-C₁₀) alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy and halo(C₁-C₁₀)alkoxy, and
R² is a hydrogen atom.

30. The process of claim 29 wherein Y² is chloro or iodo, R¹² is (C₁-C₁₀)alkyl and Z³ is (C₁-C₁₀)alkyl or ar(C₁-C₆)alkyl.

31. The process of any one of claims 25-30 wherein the suitable solvent is an ether, an ester, an alcohol, dimethylformamide, dimethylsulfoxide, acetonitrile, a chlorinated solvent or a mixture thereof.

32. The process of claim 31 wherein the solvent is dimethylformamide or tetrahydrofuran.

33. The process of claim 31 wherein the suitable base is a metal hydride, a metal alkoxide, an alkali metal hydroxide, a tertiary amine, an alkali metal carbonate, a pyridine or mixtures thereof.

34. The process of claim 33 wherein the base is triethylamine or diisopropylethylamine.

35. The process of claim 33 wherein the reaction temperature is from about −78° C. to about 100° C.

36. The process of claim 33 wherein from about 1 to about 10 equivalents of a compound of formula (IV) are used per equivalent of a compound of formula (III).

37. The process of claim 36 wherein substantially equimolar amounts of reactants are used.

38. The process of claim 33 wherein about one equivalent of base is used per equivalent of a compound of formula (IV).

39. A process leading to a compound of formula (I) 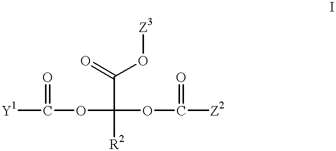
comprising the step of reacting, optionally in the presence of a suitable solvent, a compound of formula (II) 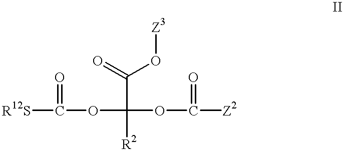
with a suitable halogenating agent wherein
Z³ is alkyl, alkylcarbonyloxyalkyl, hydroxyalkyl, alkylsulfonylalkyl, acetylaminoalkyl, haloalkyl, alkenyl, acetylaminoalkenyl, haloalkenyl, alkynyl, haloalkynyl, cycloalkyl, cycloalkenyl, carboxycycloalkyl, carboxycycloalkenyl, cycloalkylalkyl, cycloalkylalkenyl, cycloalkenylalkyl, cycloalkenylalkenyl, cycloalkylalkynyl, cycloalkenylalkynyl, carboxycycloalkylalkyl, carboxycycloalkylalkenyl, carboxycycloalkenylalkyl, carboxycycloalkenylalkenyl, carboxycycloalkylalkynyl, carboxycycloalkenylalkynyl, heterocyclyl, heterocyclylalkyl, heterocyclylalkenyl, heterocyclylalkynyl, alkoxyalkyl, alkoxyalkoxyalkyl, alkoxyalkenyl, alkoxyalkynyl, alkoxycarbonylalkyl, alkoxycarbonylalkenyl, alkoxycarbonylalkynyl, haloalkoxyalkyl, haloalkoxyalkenyl, haloalkoxyalkynyl, alkylthioalkyl, alkylthioalkenyl, alkylthioalkynyl, haloalkylthioalkyl, haloalkylthioalkenyl, haloalkylthioalkynyl, carboxyalkyl, carboxyalkenyl, carboxyalkynyl, SiR³R⁴R⁵, aryl, aryl substituted with one or more substituents independently selected from halo, nitro, hydroxy, cyano, thiocyanato, alkyl, alkylsulfonylalkyl, alkenyl, alkynyl, haloalkyl, haloalkenyl, haloalkynyl, alkoxy, haloalkoxy, SO₂NR³R⁴ and NR³R⁴, aralkyl, aralkenyl, aralkynyl, arcycloalkyl, aroxyalkyl, or aralkyl, aralkenyl, aralkynyl, arcycloalkyl, aroxyalkyl substituted with one or more substituents independently selected from halo, nitro, hydroxy, cyano, alkyl, cycloalkyl, alkenyl, alkynyl, haloalkyl, haloalkenyl, haloalkynyl, alkoxy, haloalkoxy, SO₂NR³R⁴ and NR³R⁴, heteroaryl, heteroaryl substituted with one or more substituents independently selected from halo, nitro, hydroxy, cyano, alkyl, alkenyl, alkynyl, haloalkyl, haloalkenyl, haloalkynyl, alkoxy, haloalkoxy, SO₂NR³R⁴ and NR³R⁴, heteroaralkyl, heteroaralkenyl, heteroaralkynyl, or heteroaralkyl, heteroaralkenyl, heteroaralkynyl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴,
Z² is a hydrogen atom, alkyl, alkylcarbonyloxyalkyl, alkylcarbonyl, alkenylcarbonyl, alkynylcarbonyl, hydroxyalkyl, alkylsulfonylalkyl, acetylaminoalkyl, haloalkyl, alkenyl, acetylaminoalkenyl, haloalkenyl, alkynyl, haloalkynyl, cycloalkyl, cycloalkenyl, carboxycycloalkyl, carboxycycloalkenyl, cycloalkylalkyl, cycloalkylalkenyl, cycloalkenylalkyl, cycloalkenylalkenyl, cycloalkylalkynyl, cycloalkenylalkynyl, carboxycycloalkylalkyl, carboxycycloalkylalkenyl, carboxycycloalkenylalkyl, carboxycycloalkenylalkenyl, carboxycycloalkylalkynyl, carboxycycloalkenylalkynyl, heterocyclyl, heterocyclylalkyl, heterocyclylalkenyl, heterocyclylalkynyl, alkoxyalkyl, alkoxyalkoxyalkyl, alkoxyalkenyl, alkoxyalkynyl, alkoxycarbonylalkyl, alkoxycarbonylalkenyl, alkoxycarbonylalkynyl, haloalkoxyalkyl, haloalkoxyalkenyl, haloalkoxyalkynyl, alkylthioalkyl, alkylthioalkenyl, alkylthioalkynyl, haloalkylthioalkyl, haloalkylthioalkenyl, haloalkylthioalkynyl, NR³R⁴, SO₂NR³R⁴, carboxyalkyl, carboxyalkenyl, carboxyalkynyl, dialkoxyphosphorylalkyl, aryl, aryl substituted with one or more substituents independently selected from halo, nitro, hydroxy, cyano, thiocyanato, alkyl, alkylsulfonylalkyl, alkenyl, alkynyl, haloalkyl, haloalkenyl, haloalkynyl, alkoxy, haloalkoxy, SO₂NR³R⁴ and NR³R⁴, aralkyl, aralkenyl, aralkynyl, arcycloalkyl, aroxyalkyl, or aralkyl, aralkenyl, aralkynyl, arcycloalkyl, aroxyalkyl substituted with one or more substituents independently selected from halo, nitro, hydroxy, cyano, alkyl, cycloalkyl, alkenyl, alkynyl, haloalkyl, haloalkenyl, haloalkynyl, alkoxy, haloalkoxy, SO₂NR³R⁴ and NR³R⁴, heteroaryl, heteroaryl substituted with one or more substituents independently selected from halo, nitro, hydroxy, cyano, alkyl, alkenyl, alkynyl, haloalkyl, haloalkenyl, haloalkynyl, alkoxy, haloalkoxy, SO₂NR³R⁴ and NR³R⁴, heteroaralkyl, heteroaralkenyl, heteroaralkynyl, or heteroaralkyl, heteroaralkenyl, heteroaralkynyl substituted with one or more substituents independently selected from halo, hydroxy, nitro, cyano, alkyl, alkenyl, alkynyl, haloalkyl, haloalkenyl, haloalkynyl, alkoxy, haloalkoxy, SO₂NR³R⁴ and NR³R⁴, alkylcarbonylalkyl, alkenylcarbonylalkyl, alkynylcarbonylalkyl, heterocyclylcarbonyl, heterocyclylcarbonylalkyl, heterocyclyloxycarbonylalkyl, arylcarbonyl, arylcarbonylalkyl, aralkylcarbonyl, aralkylcarbonylalkyl, aroxycarbonylalkyl, aralkoxycarbonylalkyl, heteroarylcarbonyl, heteroarylcarbonylalkyl, heteroaroxycarbonylalkyl, or arylcarbonyl, arylcarbonylalkyl, aralkylcarbonyl, aralkylcarbonylalkyl, aroxycarbonylalkyl, aralkoxycarbonylalkyl, heteroarylcarbonyl, heteroarylcarbonylalkyl, heteroaroxycarbonylalkyl substituted with one or more substituents independently selected from halo, hydroxy, nitro, cyano, alkyl, alkenyl, alkynyl, haloalkyl, haloalkenyl, haloalkynyl, alkoxy, haloalkoxy, SO₂NR³R⁴ and NR³R⁴, and C(═N—G²²)R²,
G²² is OR³, OCOR³, S(O)ⱼR³, OS(O)ⱼR³, NR³R⁴, OSO₂NR³R⁴, OP(═O)OR³NR³R⁴, OP(═O)(OR³)₂ or N═CR³R⁴,
j is 0, 1 or 2,
R² is a hydrogen atom, alkyl, alkenyl, alkynyl, alkoxyalkyl, alkoxyalkenyl, alkoxyalkynyl, alkylthioalkyl, alkylthioalkenyl, alkylthioalkynyl, carboxy, a carboxylate salt, carboxyalkyl, carboxyalkenyl, carboxyalkynyl, alkoxycarbonyl, alkoxycarbonylalkyl, alkoxycarbonylalkenyl, alkoxycarbonylalkynyl, alkylcarbonyl, alkenylcarbonyl, alkynylcarbonyl, cycloalkyl, cycloalkenyl, cycloalkylalkyl, cycloalkenylalkyl, cycloalkylalkenyl, cycloalkenylalkenyl, cycloalkylalkynyl, cycloalkenylalkynyl, heterocyclyl, heterocyclylalkyl, heterocyclylalkenyl, heterocyclylalkynyl, or alkyl, alkenyl, alkynyl, alkoxyalkyl, alkoxyalkenyl, alkoxyalkynyl, alkylthioalkyl, alkylthioalkenyl, alkylthioalkynyl, carboxyalkyl, carboxyalkenyl, carboxyalkynyl, alkoxycarbonyl, alkoxycarbonylalkyl, alkoxycarbonylalkenyl, alkoxyearbonylalkynyl, alkylcarbonyl, alkenylcarbonyl, alkynylcarbonyl, cycloalkyl, cycloalkenyl, cycloalkylalkyl, cycloalkenylalkyl, cycloalkylalkenyl, cycloalkenylalkenyl, cycloalkylalkynyl, cycloalkenylalkynyl, heterocyclyl, heterocyclylalkyl, heterocyclylalkenyl, heterocyclylalkynyl substituted with one or more substituents independently selected from halo, cyano, hydroxy, nitro, SO₂NR³R⁴ and NR³R⁴, aryl or aryl substituted with one or more substituents independently selected from halo, hydroxy, cyano, nitro, alkyl, alkenyl, alkynyl, haloalkyl, haloalkenyl, haloalkynyl, alkoxy, haloalkoxy, carboxy, alkoxycarbonyl, SO₂NR³R⁴ and NR³R⁴, aralkyl, aralkenyl, aralkynyl or aralkyl, aralkenyl, aralkynyl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, arylcarbonyl, aralkylcarbonyl, aralkenylcarbonyl, aralkynylcarbonyl or arylcarbonyl, aralkylcarbonyl, aralkenylcarbonyl, aralkynylcarbonyl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, heteroaryl or heteroaryl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, heteroaralkyl, heteroaralkenyl, heteroaralkynyl or heteroaralkyl, heteroaralkenyl, heteroaralkynyl substituted with one or more substituents independently selected from halo, cyano, hydroxy, nitro, alkyl, alkenyl, alkynyl, haloalkyl, haloalkenyl, haloalkynyl, alkoxy, haloalkoxy, SO₂NR³R⁴ and NR³R⁴, heteroaralkylcarbonyl, heteroaralkenylcarbonyl, heteroaralkynylcarbonyl or heteroaralkylcarbonyl, heteroaralkenylcarbonyl, heteroaralkynylcarbonyl substituted with one or more substituents independently selected from halo, cyano, hydroxy, nitro, alkyl, alkenyl, alkynyl, haloalkyl, haloalkenyl, haloalkynyl, alkoxy, haloalkoxy, SO₂NR³R⁴ and NR³R⁴,
R¹² is alkyl, cycloalkyl, cycloalkenyl, cycloalkylalkyl, cycloalkylalkenyl, cycloalkylalkynyl, cycloalkenylalkyl, cycloalkenylalkenyl, cycloalkenylalkynyl, carboxyalkyl, carboxyalkenyl, carboxyalkynyl, heterocyclyl, heterocyclylalkyl, heterocyclylalkenyl, heterocyclylalkynyl, alkoxyalkyl, alkenyl, alkynyl, or alkyl, cycloalkyl, cycloalkenyl, cycloalkylalkyl, cycloalkylalkenyl, cycloalkylalkynyl, cycloalkenylalkyl, cycloalkenylalkenyl, cycloalkenylalkynyl, carboxyalkyl, carboxyalkenyl, carboxyalkynyl, heterocyclyl, heterocyclylalkyl, heterocyclylalkenyl, heterocyclylalkynyl, alkoxyalkyl, alkenyl or alkynyl substituted with one or more halo, aryl or aryl substituted with one or more substituents independently selected from halo, nitro, cyano, haloalkyl, haloalkenyl, haloalkynyl, alkylcarbonyl, alkenylcarbonyl, alkynylcarbonyl, arylcarbonyl, alkylsulfonyl and arylsulfonyl, aralkyl, aralkenyl, aralkynyl, or aralkyl, aralkenyl, aralkynyl substituted with one or more substituents independently selected from halo, alkyl, alkenyl, alkynyl, haloalkyl, haloalkenyl, haloalkynyl, alkoxy and haloalkoxy, heteroaryl, heteroaralkyl, heteroaralkenyl, heteroaralkynyl, or heteroaryl, heteroaralkyl, heteroaralkenyl, heteroaralkynyl substituted with one or more substituents independently selected from halo, alkyl, alkenyl, alkynyl, haloalkyl, haloalkenyl, haloalkynyl, alkoxy and haloalkoxy,
R³, R⁴ and R⁵ are each independently a hydrogen atom, alkyl, cycloalkyl, cycloalkenyl, cycloalkylalkyl, cycloalkylalkenyl, cycloalkylalkynyl, cycloalkenylalkyl, cycloalkenylalkenyl, cycloalkenylalkynyl, carboxyalkyl, carboxyalkenyl, carboxyalkynyl, heterocyclyl, heterocyclylalkyl, heterocyclylalkenyl, heterocyclylalkynyl, alkoxyalkyl, alkenyl, alkynyl, or alkyl, cycloalkyl, cycloalkenyl, cycloalkylalkyl, cycloalkylalkenyl, cycloalkylalkynyl, cycloalkenylalkyl, cycloalkenylalkenyl, cycloalkenylalkynyl, carboxyalkyl, carboxyalkenyl, carboxyalkynyl, heterocyclyl, heterocyclylalkyl, heterocyclylalkenyl, heterocyclylalkynyl, alkoxyalkyl, alkenyl or alkynyl substituted with one or more halo, aryl, aralkyl, aralkenyl, aralkynyl, or aryl, aralkyl, aralkenyl, aralkynyl substituted with one or more substituents independently selected from halo, alkyl, alkenyl, alkynyl, haloalkyl, haloalkenyl, haloalkynyl, alkoxy and haloalkoxy, heteroaryl, heteroaralkyl, heteroaralkenyl, heteroaralkynyl, or heteroaryl, heteroaralkyl, heteroaralkenyl, heteroaralkynyl substituted with one or more substituents independently selected from halo, alkyl, alkenyl, alkynyl, haloalkyl, haloalkenyl, haloalkynyl, alkoxy and haloalkoxy, or R³ and R⁴ taken together with the nitrogen atom to which they are attached form a 5-or 6-membered saturated or unsaturated heterocyclic ring, and
Y¹ is halo.

40. The process of claim 39 wherein
Y¹ is chloro,
R¹² is alkyl, aralkyl, aryl, heteroaryl, or aralkyl, aryl or heteroaryl substituted with one or more substituents independently selected from halo, alkyl, alkenyl, alkynyl, haloalkyl, haloalkenyl, haloalkynyl, alkoxy and haloalkoxy,
Z³ is alkyl, aryl, aralkyl, or aryl or aralkyl substituted with one or more substituents independently selected from halo, alkyl, alkenyl, alkynyl, haloalkyl, haloalkenyl, haloalkynyl, alkoxy and haloalkoxy, and
R² is a hydrogen atom.

41. The process of claim 40 wherein R¹² is alkyl and Z³ is alkyl or aralkyl.

42. The process of claim 39 wherein
Z³ is (C₁-C₂₀)alkyl, (C₁-C₁₀)alkylcarbonyloxy(C₁-C₁₀)alkyl, hydroxy(C₁-C₂₀) alkyl, (C₁-C₁₀)alkylsulfonyl(C₁-C₁₀)alkyl, acetylamino(C₁-C₁₀)alkyl, halo (C₁-C₂₀) alkyl, (C₂-C₁₀)alkenyl, acetylamino(C₂-C₁₀)alkenyl, halo(C₂-C₁₀)alkenyl, (C₂-C₁₀) alkynyl, halo(C₂-C₁₀)alkynyl, cyclo(C₃-C₈)alkyl, cyclo(C₃-C₈)alkenyl, carboxycyclo(C₃-C₈)alkyl, carboxycyclo(C₃-C₈)alkenyl, cyclo(C₃-C₈)alkyl(C₁-C₁₀) alkyl, cyclo(C₃-C₈)alkyl(C₂-C₁₀)alkenyl, cyclo(C₃-C₈)alkenyl(C₁-C₁₀)alkyl, cyclo(C₃-C₈)alkenyl(C₂-C₁₀)alkenyl, cyclo(C₃-C₈)alkyl(C₂-C₁₀)alkynyl, cyclo (C₃-C₈)alkenyl(C₂-C₁₀)alkynyl, carboxycyclo(C₃-C₈)alkyl(C₁-C₁₀)alkyl, carboxycyclo(C₃-C₈)alkyl(C₂-C₁₀)alkenyl, carboxycyclo(C₃-C₈)alkenyl(C₁-C₁₀)alkyl, carboxycyclo(C₃-C₈)alkenyl(C₂-C₁₀)alkenyl, carboxycyclo(C₃-C₈)alkyl(C₂-C₁₀) alkynyl, carboxycyclo(C₃-C₈)alkenyl(C₂-C₁₀)alkynyl, heterocyclyl, heterocyclyl(C₁-C₁₀)alkyl, heterocyclyl(C₂-C₁₀)alkenyl, heterocyclyl(C₂-C₁₀) alkynyl, (C₁-C₁₀)alkoxy(C₁-C₁₀)alkyl, (C₁-C₁₀)alkoxy(C₁-C₁₀)alkoxy(C₁-C₁₀) alkyl, (C₁-C₁₀)alkoxy(C₂-C₁₀)alkenyl, (C₁-C₁₀)alkoxy(C₂-C₁₀)alkynyl, (C₁-C₁₀) alkoxycarbonyl(C₁-C₁₀)alkyl, (C₁-C₁₀)alkoxycarbonyl(C₂-C₁₀)alkenyl, (C₁-C₁₀) alkoxycarbonyl(C₂-C₁₀)alkynyl, halo(C₁-C₁₀)alkoxy(C₁-C₁₀)alkyl, halo(C₁-C₁₀) alkoxy(C₂-C₁₀)alkenyl, halo(C₁-C₁₀)alkoxy(C₂-C₁₀)alkynyl, (C₁-C₁₀) alkylthio(C₁-C₁₀)alkyl, (C₁-C₁₀)alkylthio(C₂-C₁₀)alkenyl, (C₁-C₁₀)alkylthio (C₂-C₁₀)alkynyl, halo(C₁-C₁₀)alkylthio(C₁-C₁₀)alkyl, halo(C₁-C₁₀)alkylthio(C₂-C₁₀) alkenyl, halo(C₁-C₁₀)alkylthio(C₂-C₁₀)alkynyl, carboxy(C₁-C₂₀)alkyl, carboxy(C₂-C₁₀)alkenyl, carboxy(C₂-C₁₀)alkynyl, SiR³R⁴R⁵, aryl, aryl substituted with one or more substituents independently selected from halo, nitro, hydroxy, cyano, thiocyanato, (C₁-C₁₀)alkyl, (C₁-C₁₀)alkylsulfonyl(C₁-C₁₀)alkyl, (C₂-C₁₀) alkenyl, (C₂-C₁₀)alkynyl, halo(C₁-C₁₀)alkyl, halo(C₂-C₁₀)alkenyl, halo(C₂-C₁₀) alkynyl, (C₁-C₁₀)alkoxy, halo(C₁-C₁₀)alkoxy, SO₂NR³R⁴ and NR³R⁴, ar(C₁-C₁₀) alkyl, ar(C₂-C₁₀)alkenyl, ar(C₂-C₁₀)alkynyl, arcyclo(C₃-C₈)alkyl, aroxy(C₁-C₁₀) alkyl, or ar(C₁-C₁₀)alkyl, ar(C₂-C₁₀)alkenyl, ar(C₂-C₁₀)alkynyl, arcyclo(C₃-C₈) alkyl, aroxy(C₁-C₁₀)alkyl substituted with one or more substituents independently selected from halo, nitro, hydroxy, cyano, (C₁-C₁₀)alkyl, cycloalkyl, (C₂-C₁₀)alkenyl, (C₂-C₁₀)alkynyl, halo(C₁-C₁₀)alkyl, halo(C₂-C₁₀)alkenyl, halo (C₂-C₁₀)alkynyl, (C₁-C₁₀)alkoxy, halo(C₁-C₁₀)alkoxy, SO₂NR³R⁴ and NR³R⁴, heteroaryl, heteroaryl substituted with one or more substituents independently selected from halo, nitro, hydroxy, cyano, (C₁-C₁₀)alkyl, (C₂-C₁₀)alkenyl, (C₂-C₁₀) alkynyl, halo(C₁-C₁₀)alkyl, halo(C₂-C₁₀)alkenyl, halo(C₂-C₁₀)alkynyl, (C₁-C₁₀) alkoxy, halo(C₁-C₁₀)alkoxy, SO₂NR³R⁴ and NR³R⁴, heteroar(C₁-C₁₀)alkyl, heteroar(C₂-C₁₀)alkenyl, heteroar(C₂-C₁₀)alkynyl, or heteroar(C₁-C₁₀)alkyl, heteroar(C₂-C₁₀)alkenyl, heteroar(C₂-C₁₀)alkynyl substituted with one or more substituents independently selected from halo, hydroxy, cyano, nitro, (C₁-C₁₀) alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴,
Z² is a hydrogen atom, (C₁-C₂₀)alkyl, (C₁-C₁₀)alkylcarbonyloxy(C₁-C₁₀) alkyl, (C₁-C₂₀)alkylcarbonyl, (C₁-C₂₀)alkenylcarbonyl, (C₁-C₂₀) alkynylcarbonyl, hydroxy(C₁-C₂₀)alkyl, (C₁-C₁₀)alkylsulfonyl(C₁-C₁₀)alkyl, acetylamino(C₁-C₁₀)alkyl, halo(C₁-C₂₀)alkyl, (C₂-C₂₀)alkenyl, halo(C₂-C₂₀)alkenyl, acetylamino(C₂-C₁₀)alkenyl, (C₂-C₂₀)alkynyl, halo(C₂-C₂₀)alkynyl, cyclo(C₃-C₈) alkyl, cyclo(C₃-C₈)alkenyl, carboxycyclo(C₃-C₈)alkyl, carboxycyclo(C₃-C₈) alkenyl, cyclo(C₃-C₈)alkyl(C₁-C₁₀)alkyl, cyclo(C₃-C₈)alkyl(C₂-C₁₀)alkenyl, cyclo(C₃-C₈)alkenyl(C₁-C₁₀)alkyl, cyclo(C₃-C₈)alkenyl(C₂-C₁₀)alkenyl, cyclo(C₃-C₈) alkyl(C₂-C₁₀)alkynyl, cyclo(C₃-C₈)alkenyl(C₂-C₁₀)alkynyl, carboxycyclo(C₃-C₈) alkyl(C₁-C₁₀)alkyl, carboxy(C₃-C₈)cycloalkyl(C₂-C₁₀)alkenyl, carboxycyclo(C₃-C₈) alkenyl(C₁-C₁₀)alkyl, carboxycyclo(C₃-C₈)alkenyl(C₂-C₁₀)alkenyl, carboxycyclo(C₃-C₈)alkyl(C₂-C₁₀)alkynyl, carboxycyclo(C₃-C₈)alkenyl(C₂-C₁₀) alkynyl, heterocyclyl, heterocyclyl(C₁-C₁₀)alkyl, heterocyclyl(C₂-C₁₀)alkenyl, heterocyclyl(C₂-C₁₀)alkynyl, (C₁ -C₁₀)alkoxy(C₁-C₁₀)alkyl, (C₁-C₅)alkoxy(C₁-C₅) alkoxy(C₁-C₁₀)alkyl, (C₁-C₁₀)alkoxy(C₂-C₁₀)alkenyl, (C₁-C₁₀)alkoxy(C₂-C₁₀) alkynyl, (C₁-C₁₀)alkoxycarbonyl(C₁-C₁₀)alkyl, (C₁-C₁₀)alkoxycarbonyl(C₂-C₁₀) alkenyl, (C₁-C₁₀)alkoxycarbonyl(C₂-C₁₀)alkynyl, halo(C₁-C₁₀)alkoxy(C₁-C₁₀) alkyl, halo(C₁-C₁₀)alkoxy(C₂-C₁₀)alkenyl, halo(C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀) alkylthio(C₂-C₁₀)alkynyl, halo(C₁-C₁₀)alkylthio(C₁-C₁₀)alkyl, halo(C₁-C₁₀) alkylthio(C₂-C₁₀)alkenyl, halo(C₁-C₁₀)alkylthio(C₂-C₁₀)alkynyl, SO₂NR³R⁴, NR³R⁴, carboxy(C₁-C₂₀)alkyl, carboxy(C₂-C₂₀)alkenyl, carboxy(C₂-C₂₀)alkynyl, di(C₁-C₁₀)alkoxyphosphoryl(C₁-C₁₀)alkyl, aryl, aryl substituted with one or more substituents independently selected from halo, nitro, cyano, hydroxy, (C₁-C₁₀) alkyl, (C₁-C₁₀)alkylsulfonyl(C₁-C₁₀)alkyl, (C₁-C₁₀)alkylsulfonyl, thiocyanato, (C₂-C₁₀)alkenyl, (C₂-C₁₀)alkynyl, halo(C₁-C₁₀)alkyl, halo(C₂-C₁₀)alkenyl, halo( C₂-C₁₀)alkynyl, (C₁-C₁₀)alkoxy, halo(C₁-C₁₀)alkoxy, SO₂NR³R⁴, and NR³R⁴, ar (C₁-C₁₀)alkyl, ar(C₂-C₁₀)alkenyl, ar(C₂-C₁₀)alkynyl, arcyclo(C₃-C₈)alkyl, aroxy (C₁-C₁₀)alkyl, or ar(C₁-C₁₀)alkyl, ar(C₂-C₁₀)alkenyl, ar(C₂-C₁₀)alkynyl, arcyclo (C₃-C₈)alkyl, aroxy(C₁-C₁₀)alkyl substituted with one or more substituents independently selected from halo, nitro, hydroxy, cyano, (C₁-C₁₀)alkyl, cyclo(C₃-C₈) alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroaryl, heteroaryl substituted with one or more substituents independently selected from halo, hydroxy, nitro, cyano, (C₁-C₁₀)alkyl, (C₂-C₁₀)alkenyl, (C₂-C₁₀) alkynyl, halo(C₁-C₁₀)alkyl, halo(C₂-C₁₀)alkenyl, halo(C₂-C₁₀)alkynyl, (C₁-C₁₀) alkoxy, halo(C₁-C₁₀)alkoxy and NR³R⁴, heteroar(C₁-C₁₀)alkyl, heteroar(C₂-C₁₀) alkenyl, heteroar(C₂-C₁₀)alkynyl, or heteroar(C₁-C₁₀)alkyl, heteroar(C₂-C₁₀) alkenyl, heteroar(C₂-C₁₀)alkynyl substituted with one or more substituents independently selected from halo, hydroxy, cyano, nitro, (C₁-C₁₀)alkyl, (C₂-C₁₀) alkenyl, (C₂-C₁₀)alkynyl, halo(C₁-C₁₀)alkyl, halo(C₂-C₁₀)alkenyl, halo(C₂-C₁₀) alkynyl, (C₁-C₁₀)alkoxy, halo(C₁-C₁₀)alkoxy, SO₂NR³R⁴ and NR³R⁴, (C₁-C₁₀)alkylcarbonyl(C₁-C₁₀)alkyl, (C₂-C₁₀)alkenylcarbonyl(C₁-C₁₀)alkyl, (C₂-C₁₀)alkynylcarbonyl(C₁-C₁₀)alkyl, heterocyclylcarbonyl, heterocyclylcarbonyl(C₁-C₁₀)alkyl, heterocyclyloxycarbonyl(C₁-C₁₀)alkyl, arylcarbonyl, arylcarbonyl(C₁-C₁₀) alkyl, ar(C₁-C₁₀)alkylcarbonyl, ar(C₁-C₁₀)alkylcarbonyl(C₁-C₁₀)alkyl, aroxycarbonyl(C₁-C₁₀)alkyl, ar(C₁-C₁₀)alkoxycarbonyl(C₁-C₁₀)alkyl, heteroarylcarbonyl, heteroarylcarbonyl(C₁-C₁₀)alkyl, heteroaroxycarbonyl(C₁-C₁₀) alkyl, or arylcarbonyl, arylcarbonyl(C₁-C₁₀)alkyl, ar(C₁-C₁₀)alkylcarbonyl, ar(C₁-C₁₀)alkylcarbonyl(C₁-C₁₀)alkyl, aroxycarbonyl(C₁-C₁₀)alkyl, ar(C₁-C₁₀) alkoxycarbonyl(C₁-C₁₀)alkyl, heteroarylcarbonyl, heteroarylcarbonyl(C₁-C₁₀) alkyl, heteroaroxycarbonyl(C₁-C₁₀)alkyl substituted with one or more substituents independently selected from halo, hydroxy, nitro, cyano, (C₁-C₁₀) alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, and C(═N—G22)R²,
G²² is OR³, OCOR³, S(O)ⱼR³, OS(O)ⱼR³, NR³R⁴, OSO₂NR³R⁴, OP(═O)OR³NR³R⁴, OP(═O)(OR³)₂ or N═CR³R⁴,
j is 0, 1 or 2,
R² is a hydrogen atom, (C₁-C₂₀)alkyl, (C₂-C₁₀)alkenyl, (C₂-C₁₀)alkynyl, (C₁-C₁₀) alkoxy(C₁-C₁₀)alkyl, (C₁-C₁₀)alkoxy(C₂-C₁₀)alkenyl, (C₁-C₁₀)alkoxy(C₂-C₁₀) alkynyl, (C₁-C₁₀)alkylthio(C₁-C₁₀)alkyl, (C₁-C₁₀)alkylthio(C₂-C₁₀)alkenyl, (C₁-C₁₀) alkylthio(C₂-C₁₀)alkynyl, carboxy, a carboxylate salt, carboxy(C₁-C₂₀)alkyl, carboxy(C₂-C₂₀)alkenyl, carboxy(C₂-C₂₀)alkynyl, (C₁-C₂₀)alkoxycarbonyl, (C₁-C₁₀) alkoxycarbonyl(C₁-C₁₀)alkyl, (C₁-C₁₀)alkoxycarbonyl(C₂-C₁₀)alkenyl, (C₁-C₁₀) alkoxycarbonyl(C₂-C₁₀)alkynyl, (C₁-C₂₀)alkylcarbonyl, (C₂-C₂₀) alkenylcarbonyl, (C₂-C₂₀)alkynylcarbonyl, cyclo(C₃-C₈)alkyl, cyclo(C₃-C₈) alkenyl, cyclo(C₃-C₈)alkyl(C₁-C₁₀)alkyl, cyclo(C₃-C₈)alkenyl(C₁-C₁₀)alkyl, cyclo(C₃-C₈)alkyl(C₂-C₁₀)alkenyl, cyclo(C₃-C₈)alkenyl(C₂-C₁₀)alkenyl, cyclo(C₃-C₈) alkyl(C₂-C₁₀)alkynyl, cyclo(C₃-C₈)alkenyl(C₂-C₁₀)alkynyl, heterocyclyl, heterocyclyl(C₁-C₁₀)alkyl, heterocyclyl(C₂-C₁₀)alkenyl, heterocyclyl(C₂-C₁₀) alkynyl, or (C₁-C₂₀)alkyl, (C₂-C₁₀)alkenyl, (C₂-C₁₀)alkynyl, (C₁-C₁₀)alkoxy(C₁-C₁₀) alkyl, (C₁-C₁₀)alkoxy(C₂-C₁₀)alkenyl, (C₁-C₁₀)alkoxy(C₂-C₁₀)alkynyl, (C₁-C₁₀) alkylthio(C₁-C₁₀)alkyl, (C₁-C₁₀)alkylthio(C₂-C₁₀)alkenyl, (C₁-C₁₀)alkylthio(C₂-C₁₀) alkynyl, carboxy(C₁-C₂₀)alkyl, carboxy(C₂-C₁₀)alkenyl, carboxy(C₂-C₁₀) alkynyl, (C₁-C₂₀)alkoxycarbonyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, (C₁-C₂₀) alkylcarbonyl, (C₂-C₁₀)alkenylcarbonyl, (C₂-C₁₀)alkynylcarbonyl, cyclo(C₃-C₈) alkyl, cyclo(C₃-C₈)alkenyl, cyclo(C₃-C₈)alkyl(C₁-C₁₀)alkyl, cyclo(C₃-C₈) alkenyl(C₁-C₁₀)alkyl, cyclo(C₃-C₈)alkyl(C₂-C₁₀)alkenyl, cyclo(C₃-C₈)alkenyl (C₂-C₁₀)alkenyl, cyclo(C₃-C₈)alkyl(C₂-C₁₀)alkynyl, cyclo(C₃-C₈)alkenyl(C₂-C₁₀)alkynyl, heterocyclyl, heterocyclyl(C₁-C₁₀)alkyl, heterocyclyl(C₂-C₁₀)alkenyl, heterocyclyl(C₂-C₁₀)alkynyl substituted with one or more substituents independently selected from halo, cyano, hydroxy, nitro, SO₂NR³R⁴ and NR³R⁴, aryl or aryl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, carboxy, (C₁-C₄)alkoxycarbonyl, SO₂NR³R⁴ and NR³R⁴, ar(C₁-C₁₀)alkyl, ar (C₂-C₁₀)alkenyl, ar(C₂-C₁₀)alkynyl, or ar(C₁-C₁₀)alkyl, ar(C₂-C₁₀)alkenyl, ar(C₂-C₁₀) alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, arylcarbonyl, ar(C₁-C₁₀)alkylcarbonyl, ar(C₂-C₁₀) alkenylcarbonyl, ar(C₂-C₁₀)alkynylcarbonyl or arylcarbonyl, ar(C₁-C₁₀) alkylcarbonyl, ar(C₂-C₁₀)alkenylcarbonyl, ar(C₂-C₁₀)alkynylcarbonyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀) alkyl, halo(C₂-C₁₀)alkenyl, halo(C₂-C₁₀)alkynyl, (C₁-C₁₀)alkoxy, halo(C₁-C₁₀) alkoxy, SO₂NR³R⁴ and NR³R⁴, heteroaryl, heteroaryl substituted with one or more substituents independently selected from halo, (C₁-C₁₀)alkyl, (C₂-C₁₀) alkenyl, (C₂-C₁₀)alkynyl, halo(C₁-C₁₀)alkyl, halo(C₂-C₁₀)alkenyl, halo(C₂-C₁₀) alkynyl, (C₁-C₁₀)alkoxy, halo(C₁-C₁₀)alkoxy and NR³R⁴, heteroar(C₁-C₁₀)alkyl, heteroar(C₂-C₁₀)alkenyl, heteroar(C₂-C₁₀)alkynyl or heteroar(C₁-C₁₀)alkyl, heteroar(C₂-C₁₀)alkenyl, heteroar(C₂-C₁₀)alkynyl substituted with one or more substituents independently selected from halo, (C₁-C₁₀)alkyl, (C₂-C₁₀)alkenyl, (C₂-C₁₀) alkynyl, halo(C₁-C₁₀)alkyl, halo(C₂-C₁₀)alkenyl, halo(C₂-C₁₀)alkynyl, (C₁-C₁₀) alkoxy, halo(C₁-C₁₀)alkoxy, SO₂NR³R⁴ and NR³R⁴, heteroar(C₁-C₁₀) alkylcarbonyl, heteroar(C₂-C₁₀)alkenylcarbonyl, heteroar(C₂-C₁₀) alkynylcarbonyl or heteroar(C₁-C₁₀)alkylcarbonyl, heteroar(C₂-C₁₀) alkenylcarbonyl, heteroar(C₂-C₁₀)alkynylcarbonyl substituted with one or more substituents independently selected from halo, cyano, hydroxy, nitro, (C₁-C₁₀) alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, or R¹ and R² taken together with the carbon atom to which they are attached form a 5-7 membered saturated or unsaturated ring,
R³, R⁴ and R⁵ are each independently a hydrogen atom, (C₁-C₂₀)alkyl, cyclo(C₃-C₈)alkyl, cyclo(C₃-C₈)alkenyl, cyclo(C₃-C₈)alkyl(C₁-C₁₀)alkyl, cyclo(C₃-C₈) alkyl(C₂-C₁₀)alkenyl, cyclo(C₃-C₈)alkyl(C₂-C₁₀)alkynyl, cyclo(C₃-C₈)alkenyl(C₁-C₁₀) alkyl, cyclo(C₃-C₈)alkenyl(C₂-C₁₀)alkenyl, cyclo(C₃-C₈)alkenyl(C₂-C₁₀)alkynyl, carboxy(C₁-C₂₀)alkyl, carboxy(C₂-C₁₀)alkenyl, carboxy(C₂-C₁₀)alkynyl, heterocyclyl, heterocyclyl(C₁-C₁₀)alkyl, heterocyclyl(C₂-C₁₀)alkenyl, heterocyclyl(C₂-C₁₀)alkynyl, (C₁-C₁₀)alkoxy(C₁-C₁₀)alkyl, (C₂-C₁₀)alkenyl, (C₂-C₁₀) alkynyl, or (C₁-C₁₀)alkyl, cyclo(C₃-C₈)alkyl, cyclo(C₃-C₈)alkenyl, cyclo(C₃-C₈) alkyl(C₁-C₁₀)alkyl, cyclo(C₃-C₈)alkyl(C₂-C₁₀)alkenyl, cyclo(C₃-C₈)alkyl(C₂-C₁₀) alkynyl, cyclo(C₃-C₈)alkenyl(C₁-C₁₀)alkyl, cyclo(C₃-C₈)alkenyl(C₂-C₁₀)alkenyl, cyclo(C₃-C₈)alkenyl(C₂-C₁₀)alkynyl, carboxy(C₁-C₂₀)alkyl, carboxy(C₂-C₁₀)alkenyl, carboxy(C₂-C₁₀)alkynyl, heterocyclyl, heterocyclyl(C₁-C₁₀)alkyl, heterocyclyl(C₂-C₁₀) alkenyl, heterocyclyl(C₂-C₁₀)alkynyl, (C₁-C₁₀)alkoxy(C₁-C₁₀)alkyl, (C₂-C₁₀) alkenyl or (C₂-C₁₀)alkynyl substituted with one or more halo, aryl, ar(C₁-C₁₀) alkyl, ar(C₂-C₁₀)alkenyl, ar(C₂-C₁₀)alkynyl or aryl, ar(C₁-C₁₀)alkyl, ar(C₂-C₁₀) alkenyl, ar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy and halo(C₁-C₁₀)alkoxy, heteroaryl, heteroar(C₁-C₁₀)alkyl, heteroar(C₂-C₁₀)alkenyl, heteroar(C₂-C₁₀)alkynyl or heteroaryl, heteroar(C₁-C₁₀)alkyl, heteroar(C₂-C₁₀) alkenyl, heteroar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy and halo(C₁-C₁₀)alkoxy, or R³ and R⁴ taken together with the nitrogen atom to which they are attached form a 5-or 6-membered saturated or unsaturated heterocyclic ring,
R¹² is (C₁-C₂₀)alkyl, cyclo(C₃-C₈)alkyl, cyclo(C₃-C₈)alkenyl, cyclo(C₃-C₈) alkyl(C₁-C₁₀)alkyl, cyclo(C₃-C₈)alkyl(C₂-C₁₀)alkenyl, cyclo(C₃-C₈)alkyl(C₂-C₁₀) alkynyl, cyclo(C₃-C₈)alkenyl(C₁-C₁₀)alkyl, cyclo(C₃-C₈)alkenyl(C₂-C₁₀)alkenyl, cyclo(C₃-C₈)alkenyl(C₂-C₁₀)alkynyl, carboxy(C₁-C₂₀)alkyl, carboxy(C₂-C₂₀)alkenyl, carboxy(C₂-C₂₀)alkynyl, heterocyclyl, heterocyclyl(C₁-C₁₀)alkyl, heterocyclyl(C₂-C₁₀) alkenyl, heterocyclyl(C₂-C₁₀)alkynyl, (C₁-C₁₀)alkoxy(C₁-C₁₀)alkyl, (C₂-C₂₀) alkenyl, (C₂-C₂₀)alkynyl, or (C₁-C₂₀)alkyl, cyclo(C₃-C₈)alkyl, cyclo(C₃-C₈) alkenyl, cyclo(C₃-C₈)alkyl(C₁-C₁₀)alkyl, cyclo(C₃-C₈)alkyl(C₂-C₁₀)alkenyl, cyclo(C₃-C₈)alkyl(C₂-C₁₀)alkynyl, cyclo(C₃-C₈)alkenyl(C₁-C₁₀)alkyl, cyclo (C₃-C₈)alkenyl(C₂-C₁₀)alkenyl, cyclo(C₃-C₈)alkenyl(C₂-C₁₀)alkynyl, carboxy (C₁-C₂₀)alkyl, carboxy(C₂-C₂₀)alkenyl, carboxy(C₂-C₂₀)alkynyl, heterocyclyl, heterocyclyl(C₁-C₁₀)alkyl, heterocyclyl(C₂-C₁₀)alkenyl, heterocyclyl(C₂-C₁₀) alkynyl, (C₁-C₁₀)alkoxy(C₁-C₁₀)alkyl, (C₂-C₂₀)alkenyl or (C₂-C₂₀)alkynyl substituted with one or more halo, aryl or aryl substituted with one or more substituents independently selected from halo, nitro, cyano, halo(C₁-C₁₀)alkyl, halo(C₂-C₁₀)alkenyl, halo(C₂-C₁₀)alkynyl, (C₁-C₁₀)alkylcarbonyl, (C₂-C₁₀) alkenylcarbonyl, (C₂-C₁₀)alkynylcarbonyl, arylcarbonyl, (C₁-C₁₀)alkylsulfonyl and arylsulfonyl, ar(C₁-C₁₀)alkyl, ar(C₂-C₁₀)alkenyl, ar(C₂-C₁₀)alkynyl, or ar (C₁-C₁₀)alkyl, ar(C₂-C₁₀)alkenyl, ar(C₂-C₁₀)alkynyl substituted with one or more substituents independently selected from halo, (C₁-C₁₀)alkyl, (C₂-C₁₀)alkenyl, (C₂-C₁₀) alkynyl, halo(C₁-C₁₀)alkyl, halo(C₂-C₁₀)alkenyl, halo(C₂-C₁₀)alkynyl, (C₁-C₁₀) alkoxy and halo(C₁-C₁₀)alkoxy, heteroaryl, heteroar(C₁-C₁₀)alkyl, heteroar(C₂-C₁₀)alkenyl, heteroar(C₂-C₁₀)alkynyl, or heteroaryl, heteroar(C₁-C₁₀) alkyl, heteroar(C₂-C₁₀)alkenyl, heteroar(C₂-C₁₀)alkynyl substituted with one or more substituents independently selected from halo, (C₁-C₁₀)alkyl, (C₂-C₁₀) alkenyl, (C₂-C₁₀)alkynyl, halo(C₁-C₁₀)alkyl, halo(C₂-C₁₀)alkenyl, halo(C₂-C₁₀) alkynyl, (C₁-C₁₀)alkoxy and halo(C₁-C₁₀)alkoxy, and
Y¹ is chloro.

43. The process of claim 42 wherein
R¹² is (C₁-C₂₀)alkyl, ar(C₁-C₁₀)alkyl, aryl, heteroaryl, or ar(C₁-C₁₀)alkyl, aryl or heteroaryl substituted with one or more substituents independently selected from halo, (C₁-C₁₀)alkyl, (C₂-C₁₀)alkenyl, (C₂-C₁₀)alkynyl, halo(C₁-C₁₀) alkyl, halo(C₂-C₁₀)alkenyl, halo(C₂-C₁₀)alkynyl, (C₁-C₁₀)alkoxy and halo(C₁-C₁₀)alkoxy,
Z³ is (C₁-C₂₀)alkyl, aryl, ar(C₁-C₁₀)alkyl, or aryl or ar(C₁-C₁₀)alkyl substituted with one or more substituents independently selected from halo, (C₁-C₁₀) alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy and halo(C₁-C₁₀)alkoxy, and
R² is a hydrogen atom.

44. The process of claim 43 wherein R¹² is (C₁-C₁₀)alkyl and Z³ is (C₁-C₁₀)alkyl or ar(C₁-C₆)alkyl.

45. The process of any one of claims 39-44 wherein the suitable halogenating agent is chlorine gas, thionyl chloride or sulfuryl chloride.

46. The process of claim 45 wherein the halogenating agent is sulfuryl chloride.

47. The process of claim 45 wherein the suitable solvent is an aliphatic hydrocarbon or a chlorinated solvent.

48. The process of claim 45 wherein no solvent is present.

49. The process of claim 45 wherein the reaction temperature is from about −78° C. to about 100° C.

50. The process of claim 45 wherein from about 0.9 to about 1.1 equivalents of the halogenating agent are used per equivalent of a compound of formula (II).

51. A process for the preparation of a compound of formula (I) comprising the steps of
(i) reacting a compound of formula (VII) with a compound of formula (VIII) to form a compound of formula (V) 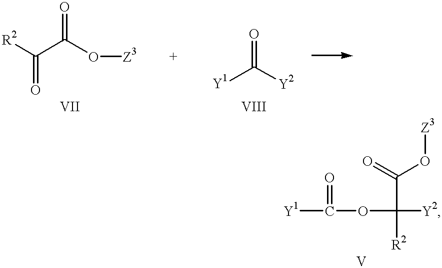
(ii) reacting a compound of formula (V) with a compound of formula (VI) to form a compound of formula (III) 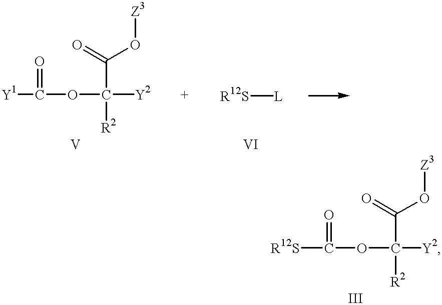
(iii) reacting a compound of formula (III) with a compound of formula (IV) to form a compound of formula (II) 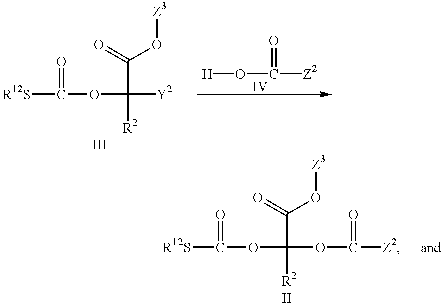
(iv) reacting a compound of formula (II) with a halogenating agent to form a compound of formula (I) 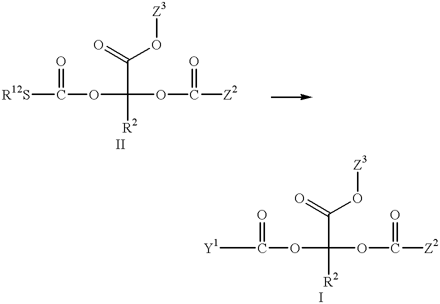
wherein
Z³ is alkyl, alkylcarbonyloxyalkyl, hydroxyalkyl, alkylsulfonylalkyl, acetylaminoalkyl, haloalkyl, alkenyl, acetylaminoalkenyl, haloalkenyl, alkynyl, haloalkynyl, cycloalkyl, cycloalkenyl, carboxycycloalkyl, carboxycycloalkenyl, cycloalkylalkyl, cycloalkylalkenyl, cycloalkenylalkyl, cycloalkenylalkenyl, cycloalkylalkynyl, cycloalkenylalkynyl, carboxycycloalkylalkyl, carboxycycloalkylalkenyl, carboxycycloalkenylalkyl, carboxycycloalkenylalkenyl, carboxycycloalkylalkynyl, carboxycycloalkenylalkynyl, heterocyclyl, heterocyclylalkyl, heterocyclylalkenyl, heterocyclylalkynyl, alkoxyalkyl, alkoxyalkoxyalkyl, alkoxyalkenyl, alkoxyalkynyl, alkoxycarbonylalkyl, alkoxycarbonylalkenyl, alkoxycarbonylalkynyl, haloalkoxyalkyl, haloalkoxyalkenyl, haloalkoxyalkynyl, alkylthioalkyl, alkylthlioalkenyl, alkylthioalkynyl, haloalkylthioalkyl, haloalkylthioalkenyl, haloalkylthioalkynyl, carboxyalkyl, carboxyalkenyl, carboxyalkynyl, SiR³R⁴R⁵, aryl, aryl substituted with one or more substituents independently selected from halo, nitro, hydroxy, cyano, thiocyanato, alkyl, alkylsulfonylalkyl, alkenyl, alkynyl, haloalkyl, haloalkenyl, haloalkynyl, alkoxy, haloalkoxy, SO₂NR³R⁴ and NR³R⁴, aralkyl, aralkenyl, aralkynyl, arcycloalkyl, aroxyalkyl, or aralkyl, aralkenyl, aralkynyl, arcycloalkyl, aroxyalkyl substituted with one or more substituents independently selected from halo, nitro, hydroxy, cyano, alkyl, cycloalkyl, alkenyl, alkynyl, haloalkyl, haloalkenyl, haloalkynyl, alkoxy, haloalkoxy, SO₂NR³R⁴ and NR³R⁴, heteroaryl, heteroaryl substituted with one or more substituents independently selected from halo, nitro, hydroxy, cyano, alkyl, alkenyl, alkynyl, haloalkyl, haloalkenyl, haloalkynyl, alkoxy, haloalkoxy, SO₂NR³R⁴ and NR³R⁴, heteroaralkyl, heteroaralkenyl, heteroaralkynyl, or heteroaralkyl, heteroaralkenyl, heteroaralkynyl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴,
Z² is a hydrogen atom, alkyl, alkylcarbonyloxyalkyl, alkylcarbonyl, alkenylcarbonyl, alkynylcarbonyl, hydroxyalkyl, alkylsulfonylalkyl, acetylaminoalkyl, haloalkyl, alkenyl, acetylaminoalkenyl, haloalkenyl, alkynyl, haloalkynyl, cycloalkyl, cycloalkenyl, carboxycycloalkyl, carboxycycloalkenyl, cycloalkylalkyl, cycloalkylalkenyl, cycloalkenylalkyl, cycloalkenylalkenyl, cycloalkylalkynyl, cycloalkenylalkynyl, carboxycycloalkylalkyl, carboxycycloalkylalkenyl, carboxycycloalkenylalkyl, carboxycycloalkenylalkenyl, carboxycycloalkylalkynyl, carboxycycloalkenylalkynyl, heterocyclyl, heterocyclylalkyl, heterocyclylalkenyl, heterocyclylalkynyl, alkoxyalkyl, alkoxyalkoxyalkyl, alkoxyalkenyl, alkoxyalkynyl, alkoxycarbonylalkyl, alkoxycarbonylalkenyl, alkoxycarbonylalkynyl, haloalkoxyalkyl, haloalkoxyalkenyl, haloalkoxyalkynyl, alkylthioalkyl, alkylthioalkenyl, alkylthioalkynyl, haloalkylthioalkyl, haloalkylthioalkenyl, haloalkylthioalkynyl, NR³R⁴, SO₂NR³R⁴, carboxyalkyl, carboxyalkenyl, carboxyalkynyl, dialkoxyphosphorylalkyl, aryl, aryl substituted with one or more substituents independently selected from halo, nitro, hydroxy, cyano, thiocyanato, alkyl, alkylsulfonylalkyl, alkenyl, alkynyl, haloalkyl, haloalkenyl, haloalkynyl, alkoxy, haloalkoxy, SO₂NR³R⁴ and NR³R⁴, aralkyl, aralkenyl, aralkynyl, arcycloalkyl, aroxyalkyl, or aralkyl, aralkenyl, aralkynyl, arcycloalkyl, aroxyalkyl substituted with one or more substituents independently selected from halo, nitro, hydroxy, cyano, alkyl, cycloalkyl, alkenyl, alkynyl, haloalkyl, haloalkenyl, haloalkynyl, alkoxy, haloalkoxy, SO₂NR³R⁴ and NR³R⁴, heteroaryl, heteroaryl substituted with one or more substituents independently selected from halo, nitro, hydroxy, cyano, alkyl, alkenyl, alkynyl, haloalkyl, haloalkenyl, haloalkynyl, alkoxy, haloalkoxy, SO₂NR³R⁴ and NR³R⁴, heteroaralkyl, heteroaralkenyl, heteroaralkynyl, or heteroaralkyl, heteroaralkenyl, heteroaralkynyl substituted with one or more substituents independently selected from halo, hydroxy, nitro, cyano, alkyl, alkenyl, alkynyl, haloalkyl, haloalkenyl, haloalkynyl, alkoxy, haloalkoxy, SO₂NR³R⁴ and NR³R⁴, alkylcarbonylalkyl, alkenylcarbonylalkyl, alkynylcarbonylalkyl, heterocyclylcarbonyl, heterocyclylcarbonylalkyl, heterocyclyloxycarbonylalkyl, arylcarbonyl, arylcarbonylalkyl, aralkylcarbonyl, aralkylcarbonylalkyl, aroxycarbonylalkyl, aralkoxycarbonylalkyl, heteroarylcarbonyl, heteroarylcarbonylalkyl, heteroaroxycarbonylalkyl, or arylcarbonyl, arylcarbonylalkyl, aralkylcarbonyl, aralkylcarbonylalkyl, aroxycarbonylalkyl, aralkoxycarbonylalkyl, heteroarylcarbonyl, heteroarylcarbonylalkyl, heteroaroxycarbonylalkyl substituted with one or more substituents independently selected from halo, hydroxy, nitro, cyano, alkyl, alkenyl, alkynyl, haloalkyl, haloalkenyl, haloalkynyl, alkoxy, haloalkoxy, SO₂NR³R⁴ and NR³R⁴, and C(═N—G²²)R²,
G²² is OR³, OCOR³, S(O)ⱼR³, OS(O)ⱼR³, NR³R⁴, OSO₂NR³R⁴, OP(═O)OR³NR³R⁴, OP(═O)(OR³)₂ or N═CR³R⁴,
j is 0, or 2,
R² is a hydrogen atom, alkyl, alkenyl, alkynyl, alkoxyalkyl, alkoxyalkenyl, alkoxyalkynyl, alkylthioalkyl, alkylthioalkenyl, alkylthioalkynyl, carboxy, a carboxylate salt, carboxyalkyl, carboxyalkenyl, carboxyalkynyl, alkoxycarbonyl, alkoxycarbonylalkyl, alkoxycarbonylalkenyl, alkoxycarbonylalkynyl, alkylcarbonyl, alkenylcarbonyl, alkynylcarbonyl, cycloalkyl, cycloalkenyl, cycloalkylalkyl, cycloalkenylalkyl, cycloalkylalkenyl, cycloalkenylalkenyl, cycloalkylalkynyl, cycloalkenylalkynyl, heterocyclyl, heterocyclylalkyl, heterocyclylalkenyl, heterocyclylalkynyl, or alkyl, alkenyl, alkynyl, alkoxyalkyl, alkoxyalkenyl, alkoxyalkynyl, alkylthioalkyl, alkylthioalkenyl, alkylthioalkynyl, carboxyalkyl, carboxyalkenyl, carboxyalkynyl, alkoxycarbonyl, alkoxycarbonylalkyl, alkoxycarbonylalkenyl, alkoxycarbonylalkynyl, alkylcarbonyl, alkenylcarbonyl, alkynylcarbonyl, cycloalkyl, cycloalkenyl, cycloalkylalkyl, cycloalkenylalkyl, cycloalkylalkenyl, cycloalkenylalkenyl, cycloalkylalkynyl, cycloalkenylalkynyl, heterocyclyl, heterocyclylalkyl, heterocyclylalkenyl, heterocyclylalkynyl substituted with one or more substituents independently selected from halo, cyano, hydroxy, nitro, SO₂NR³R⁴ and NR³R⁴, aryl or aryl substituted with one or more substituents independently selected from halo, hydroxy, cyano, nitro, alkyl, alkenyl, alkynyl, haloalkyl, haloalkenyl, haloalkynyl, alkoxy, haloalkoxy, carboxy, alkoxycarbonyl, SO₂NR³R⁴ and NR³R⁴, aralkyl, aralkenyl, aralkynyl or aralkyl, aralkenyl, aralkynyl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, arylcarbonyl, aralkylcarbonyl, aralkenylcarbonyl, aralkynylcarbonyl or arylcarbonyl, aralkylcarbonyl, aralkenylcarbonyl, aralkynylcarbonyl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, heteroaryl or heteroaryl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, heteroaralkyl, heteroaralkenyl, heteroaralkynyl or heteroaralkyl, heteroaralkenyl, heteroaralkynyl substituted with one or more substituents independently selected from halo, cyano, hydroxy, nitro, alkyl, alkenyl, alkynyl, haloalkyl, haloalkenyl, haloalkynyl, alkoxy, haloalkoxy, SO₂NR³R⁴ and NR³R⁴, heteroaralkylcarbonyl, heteroaralkenylcarbonyl, heteroaralkynylcarbonyl or heteroaralkylcarbonyl, heteroaralkenylcarbonyl, heteroaralkynylcarbonyl substituted with one or more substituents independently selected from halo, cyano, hydroxy, nitro, alkyl, alkenyl, alkynyl, haloalkyl, haloalkenyl, haloalkynyl, alkoxy, haloalkoxy, SO₂NR³R⁴ and NR³R⁴,
R¹² is alkyl, cycloalkyl, cycloalkenyl, cycloalkylalkyl, cycloalkylalkenyl, cycloalkylalkynyl, cycloalkenylalkyl, cycloalkenylalkenyl, cycloalkenylalkynyl, carboxyalkyl, carboxyalkenyl, carboxyalkynyl, heterocyclyl, heterocyclylalkyl, heterocyclylalkenyl, heterocyclylalkynyl, alkoxyalkyl, alkenyl, alkynyl, or alkyl, cycloalkyl, cycloalkenyl, cycloalkylalkyl, cycloalkylalkenyl, cycloalkylalkynyl, cycloalkenylalkyl, cycloalkenylalkenyl, cycloalkenylalkynyl, carboxyalkyl, carboxyalkenyl, carboxyalkynyl, heterocyclyl, heterocyclylalkyl, heterocyclylalkenyl, heterocyclylalkynyl, alkoxyalkyl, alkenyl or alkynyl substituted with one or more halo, aryl or aryl substituted with one or more substituents independently selected from halo, nitro, cyano, haloalkyl, haloalkenyl, haloalkynyl, alkylcarbonyl, alkenylcarbonyl, alkynylcarbonyl, arylcarbonyl, alkylsulfonyl and arylsulfonyl, aralkyl, aralkenyl, aralkynyl, or aralkyl, aralkenyl, aralkynyl substituted with one or more substituents independently selected from halo, alkyl, alkenyl, alkynyl, haloalkyl, haloalkenyl, haloalkynyl, alkoxy and haloalkoxy, heteroaryl, heteroaralkyl, heteroaralkenyl, heteroaralkynyl, or heteroaryl, heteroaralkyl, heteroaralkenyl, heteroaralkynyl substituted with one or more substituents independently selected from halo, alkyl, alkenyl, alkynyl, haloalkyl, haloalkenyl, haloalkynyl, alkoxy and haloalkoxy,
R³, R⁴ and R⁵ are each independently a hydrogen atom, alkyl, cycloalkyl, cycloalkenyl, cycloalkylalkyl, cycloalkylalkenyl, cycloalkylalkynyl, cycloalkenylalkyl, cycloalkenylalkenyl, cycloalkenylalkynyl, carboxyalkyl, carboxyalkenyl, carboxyalkynyl, heterocyclyl, heterocyclylalkyl, heterocyclylalkenyl, heterocyclylalkynyl, alkoxyalkyl, alkenyl, alkynyl, or alkyl, cycloalkyl, cycloalkenyl, cycloalkylalkyl, cycloalkylalkenyl, cycloalkylalkynyl, cycloalkenylalkyl, cycloalkenylalkenyl, cycloalkenylalkynyl, carboxyalkyl, carboxyalkenyl, carboxyalkynyl, heterocyclyl, heterocyclylalkyl, heterocyclylalkenyl, heterocyclylalkynyl, alkoxyalkyl, alkenyl or alkynyl substituted with one or more halo, aryl, aralkyl, aralkenyl, aralkynyl, or aryl, aralkyl, aralkenyl, aralkynyl substituted with one or more substituents independently selected from halo, alkyl, alkenyl, alkynyl, haloalkyl, haloalkenyl, haloalkynyl, alkoxy and haloalkoxy, heteroaryl, heteroaralkyl, heteroaralkenyl, heteroaralkynyl, or heteroaryl, heteroaralkyl, heteroaralkenyl, heteroaralkynyl substituted with one or more substituents independently selected from halo, alkyl, alkenyl, alkynyl, haloalkyl, haloalkenyl, haloalkynyl, alkoxy and haloalkoxy, or R³ and R⁴ taken together with the nitrogen atom to which they are attached form a 5-or 6-membered saturated or unsaturated heterocyclic ring,
Y¹ is halo,
Y² is halo or OCCl₃, and
L is a hydrogen atom, a sodium atom or a potassium atom.
